(19) United States
(12) Patent Application Publication
(10) Pub. No.: US 2023/0293410 A1
(43) Pub. Date: Sep. 21, 2023

(54) Compositions Comprising Lipophilic Compounds and One or More (Bio)-Alkanediols

(71) Applicant: Symrise AG, Holzminden (DE)

(72) Inventors: Nikolas Bugdahn, Holzminden (DE); Sebastian Bruncke, Höxter (DE); Vanessa Schade, Holzminden (DE); Sabine Lange, Holzminden (DE)

(21) Appl. No.: 18/301,004

(22) Filed: Apr. 14, 2023

Related U.S. Application Data
(63) Continuation of application No. 18/009,284, filed on Dec. 8, 2022.

(30) Foreign Application Priority Data
Dec. 9, 2020 (WO) PCT/EP2020/085164

(51) Int. Cl. A61K 8/34; A61K 47/10; A61K 45/06; A61Q 19/00

(52) U.S. Cl. CPC A61K 8/345; A61K 45/06; A61K 47/10; A61Q 19/00; A61K 2800/5922

(57) ABSTRACT

The present invention relates to a cosmetic or pharmaceutical, preferably dermatological, composition comprising or consisting of a liquid lipophilic component and an effective amount of 1,2-heptanediol and/or 2,3-heptanediol or of a specific alkanediol or a mixture of two or more different specific alkanediols and the use of said compositions as a cosmetic, for personal care, as a pharmaceutical or for animal care. Additionally, the present invention relates to the use of 1,2-heptanediol and/or 2,3-heptanediol or of a specific alkanediol or of a mixture of two or more different specific alkanediols for improving the sensory properties, i.e. skin sensation, of a cosmetic or pharmaceutical composition upon topical application, or as silicone replacer.

### TECHNICAL FIELD

The present invention relates to a cosmetic or pharmaceutical, preferably dermatological, composition comprising or consisting of a liquid lipophilic component and an effective amount of 1,2-heptanediol and/or 2,3-heptanediol or of a specific alkanediol or a mixture of two or more different specific alkanediols and the use of said compositions as a cosmetic, for personal care, as a pharmaceutical or for animal care. Additionally, the present invention relates to the use of 1,2-heptanediol and/or 2,3-heptanediol or of a specific alkanediol or of a mixture of two or more different specific alkanediols for improving the sensory properties, i.e. skin sensation, of a cosmetic or pharmaceutical composition upon topical application, or as silicone replacer.

### BACKGROUND ART

Besides water, silicones, liquid lipophilic components such as plant oils, hydrocarbons, fatty alcohols, fatty acid esters, and UV filters, are among the most important cosmetic or pharmaceutical basic ingredients and are frequently used for a variety of external applications. They can be used directly as skin care or hair care or body care compositions, but also as the basic substances for the manufacture of cosmetic or pharmaceutical products. Creams, body-lotions, serum, make-up, exfoliating agents, shower-gels and shampoos may all contain more or less of these components. However, when applied to the skin, the liquid lipophilic components lead to a sticky skin feel with poor absorption properties, leaving a discomfort on the skin.

Of particular relevance are silicones which are polymers with unique properties that have numerous benefits in all aspects of cosmetic formulations. Mainly their hydrophobic and conditioning properties are utilized in the cosmetics field. There are volatile and liquid polysiloxanes which facilitate the application of the cosmetic products, and there are highly molecular even wax-like representatives which remain on the skin surface. The hydrophobic effect and simultaneously velvety feeling in skin care products is appreciated. The smoothening and adhesive effect is used in cleansing products and body lotions as for instance for the re-fattening of the skin. The obviously agreeable feeling on the skin subjectively simulates a skin recovery which may not be present objectively. A broad field of application is in shampoos and hair conditioning products. Dimethyl silicone oil can be used for cosmetics production in order to improve the cosmetic properties. Silicone oil has a low surface tension combined with viscosity and can affect other components of cosmetics so that these easily spread into a thin film on the skin, thus avoiding a sticky feeling.

Despite their advantageously properties, the environmental risk of silicones, on the other hand, is troubling. These molecules are chemically inert, very stable, and that raises the question of their biodegradability. Their main drawback is that they are almost non-biodegradable, hence highly polluting for the environment.

Additionally, consumers increasingly desire for natural and renewable, i.e. sustainable components for use in compositions applicable to various industries, including cosmetic and pharmaceuticals, and particularly in personal care compositions. Hence, there is a desire to develop alternatives and to find more user-friendly chemical component combinations that are derived from sustainable and renewable resources.

Hence, there is a trend to replace silicones components in cosmetic or pharmaceutical compositions with other components, such as fats, oils or other lipids.

However, such compositions or formulations often exhibit unpleasant sensory properties when applied on the skin such as an oily, greasy feeling, imparting unwanted gloss to the skin, and rendering the composition sticky. Furthermore, they are often not easily absorbed or leave a discomfort of the absorbed product when applied on the skin.

Even though texturing agents on the basis of polyamide, nylon, silica, polymethylmethacrylate or starch are known which claim to improve the sensory properties of topical composition in general, these texturing agents commonly are employed in high amounts such as in amounts of 8% by weight or more.

WO 2014/184228 discloses a topical composition comprising a natural organic oil, which is characterized in that the composition further comprises a porous polymethylmethacrylate bead having a certain particle size and an oil absorption capacity in the range of 1.2 to 2.5 cc/g in an amount in the range of 0.1 to 5 % by weight.

However, the current interest in “natural” cosmetics has turned the attention of the cosmetic chemist back to improved versions of the classical raw materials or natural and renewable raw materials, i.e. which are sustainable.

Thus, there is still an ongoing need to further improve the complex sensory properties of topical compositions comprising a liquid lipophilic component in order to fulfill the demanding expectations on the part of the end consumers. The cosmetic or pharmaceutical industry is permanently searching for ingredients which can improve this negative aspect.

When searching for agents improving the sensory properties of topical compositions comprising a liquid lipophilic component it must be taken into account that the substances must be toxicologically acceptable, well tolerated by the skin, stable, especially in the customary cosmetic and/or pharmaceutical formulation, substantially and preferably odourless and able to be prepared inexpensively, i.e. using standard methods and/or starting from standard precursors.

However, there is no clear dependency or a predictable relationship between the chemical structure of a substance, on the one hand, and its activity towards specific liquid lipophilic components, on the other hand, which makes the search for suitable substances more difficult.

It is therefore an object of the present invention to provide a cosmetic or pharmaceutical composition, comprising or consisting of a liquid lipophilic component which has an improved skin sensation upon topical application. In particular it is an object to improve the spreading behaviour and/or to reduce fatty/greasy feel and/or to improve absorption on the skin.

It is another object of the present invention to provide a substance, which improves the complex sensory properties of topical composition comprising liquid lipophilic components.

Surprisingly it has been found that the addition of an effective amount of 1,2-heptanediol or 2,3-heptanediol or a mixture comprising 1,2-heptanediol and 2,3-heptanediol or a specific alkanediol or a mixture of two or more different specific alkanediols as defined to a topical composition comprising a liquid lipophilic component significantly improves the short-term, but also the long-term sensory properties.

### SUMMARY OF THE INVENTION

In order to accomplish the above problem, the present invention provides in a first aspect a cosmetic or pharmaceutical composition, comprising or consisting of
(a1) 1,2-heptanediol;
(b1) at least one liquid lipophilic component; and
(c1) optionally at least one cosmetically or pharmaceutically active substance and/or additive; or
(a2) 2,3-heptanediol;
(b2) at least one liquid lipophilic component; and
(c2) optionally at least one cosmetically or pharmaceutically active substance and/or additive; or
(a3) a mixture comprising 1,2-heptanediol and 2,3-heptanediol;
(b3) at least one liquid lipophilic component; and
(c3) optionally at least one cosmetically or pharmaceutically active substance and/or additive.

In a second aspect, the present invention provides a cosmetic or pharmaceutical composition, comprising or consisting of
(a) at least one linear alkanediol having a carbon chain of 5 to 14 carbon atoms or a mixture comprising at least one first linear alkanediol having a carbon chain of 5 to 14 carbon atoms and one or more second linear alkanediol having a carbon chain of 5 to 14 carbon atoms which is different from the first linear alkanediol;
(b) at least one liquid lipophilic component; and
(c) optionally at least one cosmetically or pharmaceutically active substance and/or additive.

In a further aspect, the present invention provides for the use of the cosmetic or pharmaceutical composition as a cosmetic, for personal care, in particular for skin, hair, scalp or nail care, as pharmaceutical or for animal care.

In a further aspect, the present invention provides for the use of 1,2-heptanediol or 2,3-heptanediol or a mixture comprising 1,2-heptanediol and 2,3-heptanediol or at least one linear alkanediol or a mixture comprising at least one first linear alkanediol and one or more second linear alkanediol for improving the skin sensation of a cosmetic or pharmaceutical composition comprising at least one liquid lipophilic component upon topical application.

In a still further aspect, the present invention provides for the use of 1,2-heptanediol or 2,3-heptanediol or a mixture comprising 1,2-heptanediol and 2,3-heptanediol or at least one linear alkanediol or a mixture comprising at least one first linear alkanediol and one or more second linear alkanediol as silicone replacer in a cosmetic or pharmaceutical composition comprising at least one liquid lipophilic component.

Finally, the present invention provides for the use of 1,2-heptanediol or 2,3-heptanediol or a mixture comprising 1,2-heptanediol and 2,3-heptanediol or at least one linear alkanediol or a mixture comprising at least one first linear alkanediol and one or more second linear alkanediol for preparing a cosmetic or pharmaceutical composition comprising at least one liquid lipophilic component.

### DESCRIPTION OF FIGURES

FIG. 1 shows the solubility of different alkanediols in sunflower oil: 1,2-pentanediol, 1,2-hexanediol, 1,2-heptanediol, 1,2-octanediol, and 2,3-octanediol; or mixtures of alkanediols in sunflower oil: 1,2-octanediol/ 2,3-octanediol (95 : 5) and 1,2-heptanediol/2,3-heptanediol (95 : 5).

FIG. 2 shows the spreading behavior of sunflower oil +/- 15% 1,2 heptanediol: left: sunflower oil with 15% 1,2-heptanediol; right: sunflower oil without addition of 1,2-heptanediol.

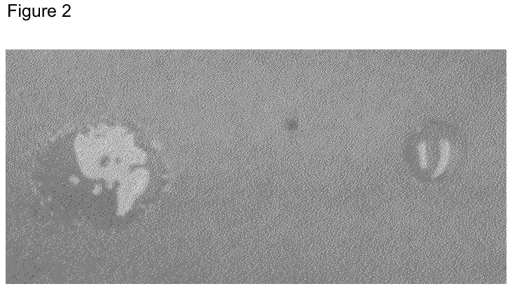

### DETAILED DESCRIPTION OF THE INVENTION

The present invention is specified in the appended claims. The invention itself, and its preferred variants, other objects and advantages, are however also apparent from the following detailed description in conjunction with the accompanying examples and figures.

In a first aspect, the present invention relates to a cosmetic or pharmaceutical composition, comprising or consisting of:
(a1) 1,2-heptanediol;
(b1) at least one liquid lipophilic component; and
(c1) optionally at least one cosmetically or pharmaceutically active substance and/or additive.

Alternatively, the present invention relates in a first aspect to a cosmetic or pharmaceutical composition, comprising or consisting of:
(a2) 2,3-heptanediol;
(b2) at least one liquid lipophilic component; and
(c2) optionally at least one cosmetically or pharmaceutically active substance and/or additive.

In a further alternative, the present invention relates in a first aspect to a cosmetic or pharmaceutical composition, comprising or consisting of:
(a3) a mixture comprising 1,2-heptanediol and 2,3-heptanediol;
(b3) at least one liquid lipophilic component; and
(c3) optionally at least one cosmetically or pharmaceutically active substance and/or additive.

In a second aspect, the present invention relates to a cosmetic or pharmaceutical composition, comprising or consisting of:
(a) at least one linear alkanediol having a carbon chain of 5 to 14 carbon atoms or a mixture comprising at least one first linear alkanediol having a carbon chain of 5 to 14 carbon atoms and one or more second linear alkanediol having a carbon chain of 5 to 14 carbon atoms which is different from the first linear alkanediol;
(b) at least one liquid lipophilic component; and
(c) optionally at least one cosmetically or pharmaceutically active substance and/or additive.

In a preferred variant of the second aspect, the present invention relates to a cosmetic or pharmaceutical composition, comprising or consisting of:
(a′) a mixture comprising at least one first linear alkanediol having a carbon chain of 5 to 14 carbon atoms and one or more second linear alkanediol having a carbon chain of 5 to 14 carbon atoms, wherein the number of the carbon atoms of the first and the second alkanediol is either same or different;
(b′) at least one liquid lipophilic component; and
(c′) optionally at least one cosmetically or pharmaceutically active substance and/or additive.

The term “comprising” means that the named components are essential, but other components may be added and is still embraced by the present invention.

The term “consisting of” as used according to the present invention means that the total amount of components (a) to (c) adds up to 100% by weight, based on the total weight of the cosmetic or pharmaceutical composition, and signifies that the subject matter is closed-ended and can only include the limitations that are expressly recited.

Whenever reference is made to “comprising” it is intended to cover both meanings as alternatives, that is the meaning can be either “comprising” or “consisting of”, unless the context dictates otherwise.

The term “at least one liquid lipophilic component” means that the cosmetic or pharmaceutical composition according to the present invention can comprise either one liquid lipophilic component or a mixture of two, three, four, five, six or even more different liquid lipophilic components.

The term “optionally” means that the subsequently described compound may but need not to be present in the composition, and that the description includes variants, were the compound is included or variants, were the compound is absent.

The term “sensory properties” in the context of the present invention is defined as the acceptance of the topic sensory attributes of a cosmetic or pharmaceutical product by consumers who are the regular users of the product category. The most important features for characterizing the sensory properties of a cosmetic or pharmaceutical formulation are the parameters “spreading” or “spreadability”, “fatty/greasy skin feel” and “absorption (remaining residue)”. Spreadability is the ease with which a product can be spread on the skin. It is commonly an important characteristic of ointments, creams, emulsions, dispersions. The parameter “fatty/greasy skin feel” is the attribute whether a skin feels fatty or greasy upon application of a cosmetic or pharmaceutical formulation. Absorption means, whether a cosmetic or pharmaceutical formulation has dermal permeability or whether a cosmetic or pharmaceutical formulation leaves a residue on the skin upon application resulting in a feeling of discomfort when applied on the skin. An improvement of the sensory properties is applicable, if at least one of the above three parameters is improved. However, it is beneficial if at least two or even three of the above three parameters are improved.

Alkanediols are glycols, i.e. any of a class of organic compounds belonging to the alcohol family; in the molecule of a glycol, two hydroxyl (—OH) groups are attached to different carbon atoms of a carbon chain.

The component (a) in the cosmetic or pharmaceutical composition according to the first aspect of the present invention is either 1,2-heptanediol (a1) or 2,3-heptanediol (a2), or a mixture comprising both heptanediols, i.e. 1,2-heptanediol and 2,3-heptanediol (a3).

1,2-heptandediol belongs to the category of alkanediols and is a straight chain alkanediol with the general formula:

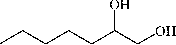

It has a carbon chain with 7 carbon atoms and the two functional OH groups of the alkanediol are in alpha,beta position and chemically bonded to the C1 and C2 carbon atoms in the alkanediol chain.

2,3-heptanediol belongs to the category of alkanediols and is a straight chain alkanediol with the general formula:

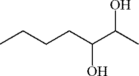

It has a carbon chain with 7 carbon atoms and the two functional OH groups of the alkanediol are in beta,gamma position and chemically bonded to the C2 and C3 carbon atoms in the alkanediol chain.

Straight chain 1,2-alkanediols have been used for more than 15 years as multifunctional actives. Short chain 1,2-alkanediols are amphiphilic compounds and thus, like 1,2-pentanediol and 1,2-hexanediol, are soluble both in water and cosmetic oils. In contrast, 1,2-octanediol is a solid and tends to precipitate or recrystallize in oily solutions. On the other hand, 1,2-decanediol is a solid and soluble only in cosmetic oils. Apart from moisturizing, some 1,2-alkanediols are used as viscosity modifiers.

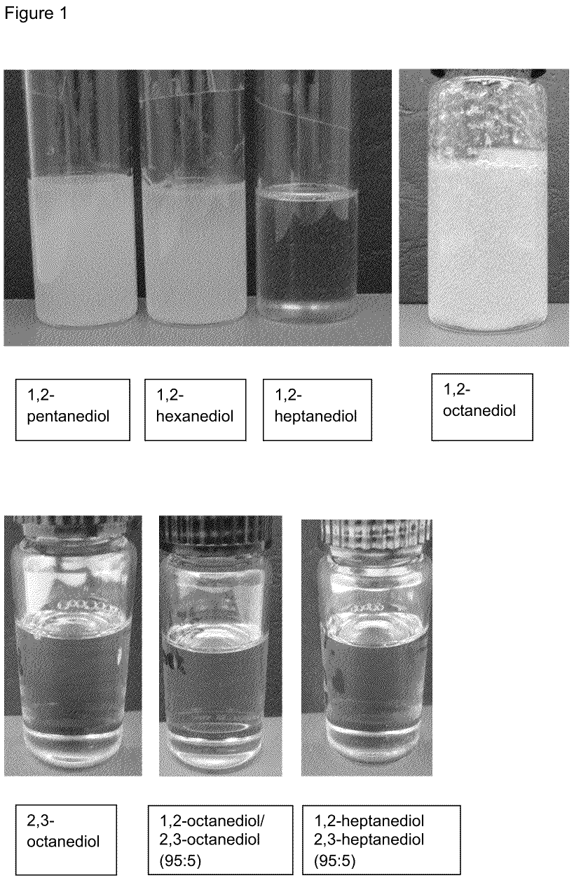

As it is demonstrated in Example 1 and showed in FIG. 1, the solubility of 1,2-heptanediol in sunflower oil is better compared to the solubility of 1,2-pentanediol and 1,2-hexanediol, the last named are well-known cosmetic ingredients. In contrast, 1,2-octanediol tends to precipitate or recrystallize in sunflower oil. Due to its excellent solubility in a lipid phase, such as natural oil, 1,2-heptanediol can be advantageously provided in high concentrations in a lipophilic component containing composition according to the first aspect of the present invention. This in turn enhances the availability of the 1,2-heptanediol and its advantageously effect in the composition.

Additionally, due to its excellent lipid phase solubility, such as oil, 1,2-heptanediol is stable in a lipophilic component containing composition, which is an important factor for the shelf-life of the lipophilic component containing composition, i.e. any consumer formulations, according to the present invention.

On the other hand, 2,3-heptanediol is liquid. Due to its excellent solubility in a lipid phase, such as natural oil, 2,3-heptanediol can be advantageously provided in high concentrations in a lipophilic component containing composition according to the first aspect of the present invention. This in turn enhances the availability of the 2,3-heptanediol and its advantageously effect in the composition.

The component (a) in the cosmetic or pharmaceutical composition according to the second aspect of the present invention is in a first alternative at least one linear alkanediol having a carbon chain of 5 to 14 carbon atoms.

Moreover, in a second alternative of this second aspect, the component (a) may include a first linear alkanediol having a carbon chain of 5 to 14 carbon atoms in combination with a second linear alkanediol having a carbon chain of 5 to 14 carbon atoms. Therefore, the invention also entails a mixture comprising a first and a second alkanediol as described herein.

Since the first linear alkanediol of the invention can be selected from the same lists and types of compounds as the linear alkanediol according to the first alternative of the second aspect, in the following, reference is made to the linear alkanediol in general, which can also be the first linear alkanediol. In mixtures of first and second linear alkanediols, where these are specifically different, the specific terms “first” and “second” will be used to distinguish the two alkanediol components.

As used in this document, the phrase “at least one linear alkanediol” or “at least one first linear alkanediol” means that the composition can comprise one linear alkanediol having a carbon chain of 5 to 14 carbon atoms or can comprise one first linear alkanediol having a carbon chain of 5 to 14 carbon atoms or can comprise more than one linear alkanediol or can comprise more than one first linear alkanediol having a carbon chain of 5 to 14 carbon atoms, i.e. two, three, four or more different linear alkanediols or first linear alkanediols having a carbon chain of 5 to 14 carbon atoms.

The linear alkanediol or the first linear alkanediol consist of a chain of 5 to 14 carbon atoms joined to each other by single covalent bonds with two OH functional groups attached to two different carbon atoms in the chain.

The at least one linear alkanediol or the at least one first linear alkanediol having a carbon chain of 5 to 14 carbon atoms in the cosmetic or pharmaceutical composition according to the second aspect of the present invention is preferably selected from the group consisting of pentanediol, hexanediol heptanediol, octanediol, nonanediol, decanediol, undecanediol, dodecanediol, tridecanediol and tetradecanediol.

In a preferred variant, the at least one linear alkanediol or the at least one first linear alkanediol is selected from the group consisting of pentanediol, hexanediol, heptanediol, octanediol, nonanediol, decanediol, undecanediol, dodecanediol and tridecanediol.

In a more preferred variant, the at least one linear alkanediol or the at least one first linear alkanediol is selected from the group consisting of alkanediols having an uneven number of carbon atoms of 5 to 13, i.e. pentanediol, heptanediol, nonanediol, undecanediol, tridecanediol and mixtures thereof.

In a still more preferred variant, the at least one linear alkanediol or the at least one first linear alkanediol is selected from the group consisting of alkanediols having an uneven number of carbon atoms of 7, 9, 11 and 13, i.e. heptanediol, nonanediol, undecanediol and tridecanediol, or is selected from the group consisting of alkanediols having an uneven number of carbon atoms of 9 and 11, i.e. nonanediol and undecanediol, or is selected from the group consisting of alkanediols having an uneven number of carbon atoms of 9 and 13, i.e. nonanediol and tridecanediol, or is selected from the group consisting of alkanediols having an uneven number of carbon atoms of 11 and 13, i.e. undecanediol and tridecanediol.

In a still more preferred variant, the at least one linear alkanediol or the at least one first linear alkanediol is either pentanediol, hexanediol, heptanediol or octanediol.

In a most preferred variant, the at least one linear alkanediol or the at least first linear alkanediol is heptanediol.

In an alternative, the cosmetic or pharmaceutical composition according to the second aspect of the present invention comprises a mixture comprising at least one first linear alkanediol having a carbon chain of 5 to 14 carbon atoms and one or more second linear alkanediols having a carbon chain of 5 to 14 carbon atoms which is different from the first linear alkanediol.

This means that the linear alkanediol mixture is a mixture comprising at least one first linear alkanediol having a carbon chain of 5 to 14 carbon atoms and one or more, i.e. two, three, four or more second llinear alkanediols having a carbon chain of 5 to 14 carbon atoms. In this alternative, the first linear alkanediol can be any of the alkanediols as described herein for the linear alkanediol in general and in particular for the first alternative of the second aspect.

For example, the mixture can include one first linear alkanediol and one, two, three or more second linear alkanediol; or the mixture can include two first linear alkanediols with one, two, three or more second linear alkanediol, etc. with the proviso, that in each mixture, the first linear alkanediols and the second linear alkanediols are different from each other.

The phrase “different from each other” means that the first linear alkanediols and the second linear alkanediols in the mixture are either different with regard to the length of their carbon chain, i.e. number of the carbon atoms, and/or with regard to their constitutional isomerism or with regard to their stereoisomerism.

Likewise, the number of the carbon atoms of the first linear alkanediol and the second linear alkanediol in the mixture can also be same. For example, the first linear alkanediol and the second linear alkanediol have a carbon chain of 7 carbon atoms, but the first linear alkanediol and the second linear alkanediol are different with regard to their constitutional isomerism or with regard to their stereoisomerism.

The second linear alkanediol preferably consists of a chain of 5 to 14 carbon atoms joined to each other by single covalent bonds with two OH functional groups attached to two different carbon atoms in the chain.

The at least one second linear alkanediol having a carbon chain of 5 to 14 carbon atoms in the cosmetic or pharmaceutical composition according to the second aspect of the present invention is selected from the group consisting of pentanediol, hexanediol, heptanediol, octanediol, nonanediol, decanediol, undecanediol, dodecanediol, tridecanediol and tetradecanediol.

In a preferred variant, the at least one second linear alkanediol is selected from the group consisting of pentanediol, hexanediol, heptanediol, octanediol, nonanediol, decanediol, undecanediol, dodecanediol and tridecanediol. If both the first and the second alkanediol is for example heptanediol, then the second linear alkanediol heptanediol is a different constitutional isomer or stereoisomer from the first linear alkanediol.

In a more preferred variant, the at least one second linear alkanediol is selected from the group consisting of pentanediol, hexanediol, heptanediol, octanediol and nonanediol, most preferably heptanediol, octanediol and nonanediol.

The term “alkanediol” within the context of the present invention also includes its constitutional isomers or position isomers. Constitutional isomers are compounds that have the same molecular formula and different connectivity. Position isomers, a particular form of constitutional isomerism, are structural isomers that can be viewed as differing only on the position of a functional group on a parent structure, which in this case is the position of the two alcohol functions.

Depending on the number of the carbon atoms of the carbon chain of the alkanediol, there are various position isomers of the alkanediol: (x,x+1) constitutional isomers; (x,x+2) constitutional isomers; (x,x+3) constitutional isomers; etc. and alpha,omega constitutional isomers when the alcohol functions are at the terminal ends of the carbon chain, wherein x stands for the number of the carbon atom in the alkanediol chain, to which the OH groups of the alkanediol are chemically bonded. For example: if x is 1, the two OH groups of the alkanediol are chemically bonded to the C1 and C2 carbon atoms in the alkanediol chain; if x is 2, the two OH groups of the alkanediol are chemically bonded to the C2 and C3 carbon atoms in the alkanediol chain, etc.

In the (x,x+1) constitutional isomers, the two OH functional groups are vicinal attached to two different adjacent carbon atoms in the chain. In the (x,x+2) constitutional isomers, the two OH functional groups are attached to two different carbon atoms in the chain where the two carbon atoms are separated by one C atom. In the (x,x+3) constitutional isomers, the two OH functional groups are attached to two different carbon atoms in the chain where the two carbon atoms are separated by two C atoms. In the alpha,omega constitutional isomers, the two functional groups are attached to the first C atom and to the terminal C atom.

In a preferred variant of the cosmetic or pharmaceutical composition according to the second aspect of the present invention, the linear alkanediol having a carbon chain of 5 to 14 carbon atoms, is preferably a vicinal (x,x+1) diol, selected from the group consisting of a 1,2-diol, 2,3-diol, 3,4-diol, 4,5-diol, further (x,x+1) diols, or mixtures thereof, preferably an alpha,beta 1,2 constitutional isomer.

In a preferred variant of the cosmetic or pharmaceutical composition according to the second aspect of the present invention, the first linear alkanediol and/or the second linear alkanediol having a carbon chain of 5 to 14 carbon atoms is a vicinal (x,x+1) diol, selected from the group consisting of a 1,2-diol, 2,3-diol, 3,4-diol, 4,5-diol, further (x,x+1) diols, or mixtures thereof, preferably an alpha,beta 1,2 constitutional isomer.

In a further preferred variant of the cosmetic or pharmaceutical composition according to the second aspect of the present invention, the linear alkanediol having a carbon chain of 5 to 14 carbon atoms, is preferably a non-vicinal (x,x+2) diol, selected from the group consisting of a 1,3-diol, 2,4-diol, 3,5-diol, further (x,x+2) diols, or mixtures thereof, preferably an alpha,gamma 1,3 constitutional isomer.

In a preferred variant of the cosmetic or pharmaceutical composition according to the second aspect of the present invention, the first linear alkanediol and/or the second linear alkanediol having a carbon chain of 5 to 14 carbon atoms is a non-vicinal (x,x+2) diol, selected from the group consisting of a 1,3-diol, 2,4-diol, 3,5-diol, 4,6-diol, further (x,x+2) diols, or mixtures thereof, preferably an alpha,gamma 1,3-consitutional isomer.

In a further preferred variant of the cosmetic or pharmaceutical composition according to the second aspect of the present invention, the linear alkanediol having a carbon chain of 5 to 14 carbon atoms, is preferably a non-vicinal (x,x+3) diol, selected from the group consisting of a 1,4 diol, 2,5 diol, further (x,x+3) diols, or mixtures thereof, preferably an alpha,delta 1,4 constitutional isomer.

In a preferred variant of the cosmetic or pharmaceutical composition according to the second aspect of the present invention, the first linear alkanediol and/or the second linear alkanediol having a carbon chain of 5 to 14 carbon atoms is a non-vicinal (x,x+3) diol, selected from the group consisting of a 1,4 diol, 2,5 diol, further (x,x+3) diols, or mixtures thereof, preferably an alpha,delta 1,4 constitutional isomer.

In a preferred variant of the cosmetic or pharmaceutical composition according to the second aspect of the invention, the linear alkanediol or the first linear alkanediol and/or the second linear alkanediol is preferably an alpha,omega alkanediol, more preferably, 1,7-heptanediol or 1,8-octanediol.

According to the present invention, vicinal (x,x+1) diols are most preferred, such as alpha,beta or beta,gamma or gamma,delta etc.

In a still preferred variant of the cosmetic or pharmaceutical composition according to the second aspect of the present invention, the linear alkanediol and in particular the first linear alkanediol and/or the second linear alkanediol is a 1,2-alkanediol, a 2,3-alkanediol, a 3,4-alkanediol, or mixtures thereof, more preferred 2,3-alkanediol.

The 1,2-alkanediols of the linear alkanediol, in particular the first linear alkanediol having a carbon chain of 5 to 14 carbon atoms and/or the second linear alkanediol having a carbon chain of 5 to 14 carbon atoms, can preferably be those as represented by the following formulae:
1,2-pentanediol

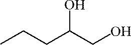

1,2-hexanediol

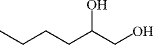

1,2-heptanediol

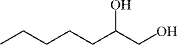

1,2-octanediol

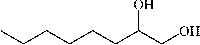

1,2-nonanediol

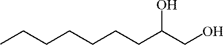

1,2-decanediol

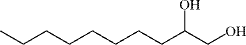

1,2-undecanediol

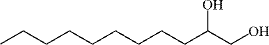

1,2-dodecanediol

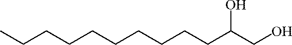

1,2-tridecanediol

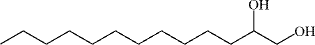

1,2-tetradecanediol

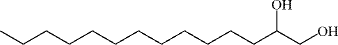 [SMILES: CCCCCCCCCCCCC(O)CO]

The 2,3-alkanediols of the linear alkanediol of the invention, in particular the first linear alkanediol having a carbon chain of 5 to 14 carbon atoms and/or the second linear alkanediol having a carbon chain of 5 to 14 carbon atoms, can preferably be those as represented by the following formulae:
2,3-pentanediol

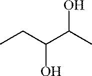

2,3-hexanediol

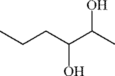

2,3-heptanediol

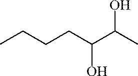

2,3-octanediol

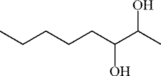

2,3-nonanediol

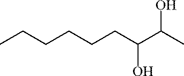

2,3-decanediol

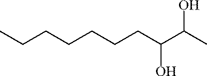

2,3-undecanediol

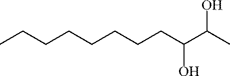

2,3-dodecanediol

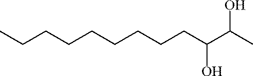

2,3-tridecanediol

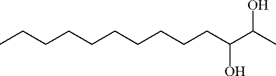

2,3-tetradecanediol

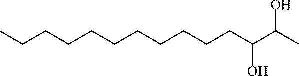 [SMILES: CCCCCCCCCCCC(O)C(C)O]

The 3,4-alkanediols of the linear alkanediol, preferably the first linear alkanediol having a carbon chain of 5 to 14 carbon atoms and/or the second linear alkanediol having a carbon chain of 5 to 14 carbon atoms, can preferably be those as represented by the following formulae:
3,4-hexanediol

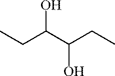

3,4-heptanediol

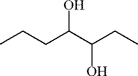

3,4-octanediol

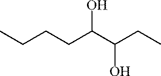

3,4-nonanediol

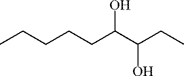

3,4-decanediol

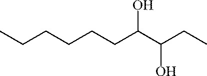

3,4-undecanediol

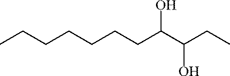

3,4-dodecanediol

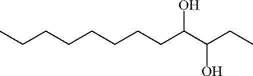

3,4-tridecanediol

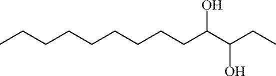

3,4-tetradecanediol

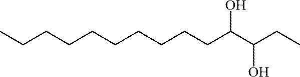 [SMILES: CCCCCCCCCCC(O)C(O)CC]

In a preferred variant, the cosmetic or pharmaceutical composition according to the second aspect of the present invention comprises a mixture comprising at least one first linear alkanediol having a carbon chain of 5 to 14 carbon atoms and one or more second linear alkanediol having a carbon chain of 5 to 14 carbon atoms wherein the number of the carbon atoms of the first and the second alkanediol is either same or different.

If both, the first and the second alkanediol have the same number of carbon atoms, such an alkanediol combination is herein also referred to as “homo alkanediol mixture” or “homo combination”. For example, the first linear alkanediol and the second linear alkanediol have a carbon chain of 7 carbon atoms, but the first linear alkanediol and the second linear alkanediol are different with regard to their constitutional isomerism or with regard to their stereoisomerism.

If the first and the second alkanediol have a different number of carbon atoms, such an alkanediol combination is herein also referred to as “hetero alkanediol mixture” or “hetero combination”. For example, the first linear alkanediol has a carbon chain of 7 carbon atoms and the second linear alkanediol has a carbon chain of 8 carbon atoms. However, beyond that, the first linear alkanediol and the second linear alkanediol can be different with regard to their constitutional isomerism or with regard to their stereoisomerism.

In a preferred variant of the cosmetic or pharmaceutical composition according to the second aspect of the present invention, the linear alkanediol or the first linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

Of the aforesaid linear alkanediols or the first linear alkanediols, the following (x,x+1) constitutional isomers are particularly preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodecanediol, 2,3-tridecanediol or mixtures thereof. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol or mixtures of said liquid alkanediols are particularly preferred. Said alkanediols can be easier incorporated into semi-finished products or final products containing a liquid lipophilic component.

In a more preferred variant, the at least one linear alkanediol or the at least one first linear alkanediol is selected from the group consisting of 1,2-alkanediols having an uneven number of carbon atoms of 5 to 13, i.e. 1,2-pentanediol, 1,2-heptanediol, 1,2-nonanediol, 1,2-undecanediol, 1,2-tridecanediol and mixtures thereof.

In a more preferred variant, the linear alkanediol or the first linear alkanediol is selected from the group consisting of 1,2-alkanediols having an uneven number of carbon atoms of 7, 9, 11 and 13, i.e. 1,2-heptanediol, 1,2-nonanediol, 1,2-undecanediol or 1,2-tridecanediol, or is selected from the group consisting of 1,2-alkanediols having an uneven number of carbon atoms of 9 and 11, i.e. 1,2-nonanediol and 1,2-undecanediol, or is selected from the group consisting of 1,2-alkanediols having an uneven number of carbon atoms of 9 and 13, i.e. 1,2-nonanediol and 1,2-tridecanediol, or is selected from the group consisting of 1,2-alkanediols having an uneven number of carbon atoms of 11 and 13, i.e. 1,2-undecanediol and 1,2-tridecanediol.

In a more preferred variant, the at least one linear alkanediol or the at least one first linear alkanediol is selected from the group consisting of 2,3-alkanediols having an uneven number of carbon atoms of 5 to 13, i.e. 2,3-pentanediol, 2,3-heptanediol, 2,3-nonanediol, 2,3-undecanediol, 2,3-tridecanediol and mixtures thereof.

In a still more preferred variant, the linear alkanediol or the first linear alkanediol is selected from the group consisting of 2,3-alkanediols having an uneven number of carbon atoms of 7, 9, 11 and 13, i.e. 2,3-heptanediol, 2,3-nonanediol, 2,3-undecanediol or 2,3-tridecanediol, or is selected from the group consisting of 2,3-alkanediols having an uneven number of carbon atoms of 9 and 11, i.e. 2,3-nonanediol and 2,3-undecanediol, or is selected from the group consisting of 2,3-alkanediols having an uneven number of carbon atoms of 9 and 13, i.e. 2,3-nonanediol and 2,3-tridecanediol, or is selected from the group consisting of 2,3-alkanediols having an uneven number of carbon atoms of 11 and 13, i.e. 2,3-undecanediol and 2,3-tridecanediol.

In a further preferred variant of the cosmetic or pharmaceutical composition according to the second aspect of the present invention, the linear alkanediol or the first linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol and mixtures thereof.

More preferred, in the cosmetic or pharmaceutical composition according to the second aspect of the present invention, the linear alkanediol or the first linear alkanediol is selected from the group consisting of 1,2-pentanediol, 2,3-pentanediol, 3,4-pentanediol and mixtures thereof or is selected from the group consisting of 1,2-heptanediol, 2,3-heptanediol, 3,4-heptanediol, and mixtures thereof. However, the linear alkanediol or the first linear alkanediol can also preferably be selected from the group consisting of 1,2-octanediol, 2,3-octanediol, 3,4-octanediol, and mixtures thereof.

Even more preferred, in the cosmetic or pharmaceutical composition according to the second aspect of the present invention, the linear alkanediol or the first linear alkanediol is an alpha,beta or a beta,gamma diol as either 1,2-heptanediol, 2,3-heptanediol, or a mixture thereof. However, the linear alkanediol or the first linear alkanediol can also preferably be an alpha,beta or a beta,gamma diol as either 1,2-octanediol, 2,3-octanediol, or a mixture thereof. A mixture comprising 1,2-heptanediol and 2,3-octanediol or a mixture of 1,2-octanediol and 2,3-heptanediol is also possible.

Most preferred, the linear alkanediol or the first linear alkanediol is 1,2-heptanediol or 1,2-octanediol.

Likewise, in a further preferred variant of the cosmetic or pharmaceutical composition according to the second aspect of the present invention, the linear alkanediol or the first linear alkanediol is selected from the group consisting of:
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol, and mixtures thereof.

More preferred, in the cosmetic or pharmaceutical composition according to the second aspect of the present invention, the linear alkanediol or the first linear alkanediol is selected from the group consisting of 1,2-nonanediol, 2,3-nonanediol, 3,4-nonanediol, and mixtures thereof, or can also be preferably selected from the group consisting of 1,2-decanediol, 2,3-decanediol, 3,4-decanediol, and mixtures thereof, or can also be preferably selected from the group consisting of 1,2-undecanediol, 2,3-undecanediol, 3,4-undecanediol, and mixtures thereof.

Even more preferred, in the cosmetic or pharmaceutical composition according to the second aspect of the present invention, the linear alkanediol or the first linear alkanediol is an alpha,beta or a beta,gamma diol as either 1,2-nonanediol, 2,3-nonanediol, or a mixture thereof. However, the linear alkanediol or the first linear alkanediol can also preferably be 1,2-decanediol, 2,3-decanediol, or a mixture thereof.

Additionally, the linear alkanediol or the first linear alkanediol can also preferably be 1,2-undecanediol, 2,3-undecanediol, or a mixture thereof. A mixture comprising 1,2-nonanediol and/or 2,3-decanediol and/or 2,3-undecanediol or a mixture of 1,2-decanediol and/or 2,3-nonanediol and/or 2,3-undecanediol or a mixture of 1,2-undecanediol and/or 2,3-nonanediol and 2,3-decanediol is also possible.

In a preferred variant of the cosmetic or pharmaceutical composition according to the second aspect of the present invention, the second linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

Of the aforesaid second linear alkanediols, the following (x,x+1) constitutional isomers are preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodedcanediol, or 2,3-tridecanediol. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol or mixtures of said liquid alkanediols are particularly preferred. Said alkanediols can be easier incorporated into semi-finished products or final products containing a liquid lipophilic component.

In a more preferred variant, the second linear alkanediol is selected from the group consisting of 1,2-alkanediols having an uneven number of carbon atoms of 5 to 13, i.e. 1,2-pentanediol, 1,2-heptanediol, 1,2-nonanediol, 1,2-undecanediol, 1,2-tridecanediol and mixtures thereof.

In a more preferred variant, the second linear alkanediol is selected from the group consisting of 1,2-alkanediols having an uneven number of carbon atoms of 7, 9, 11 and 13, i.e. 1,2-heptanediol, 1,2-nonanediol, 1,2-undecanediol and 1,2-tridecanediol, or is selected from the group consisting of 1,2-alkanediols having an uneven number of carbon atoms of 9 and 11, i.e. 1,2-nonanediol and 1,2-undecanediol, or is selected from the group consisting of 1,2-alkanediols having an uneven number of carbon atoms of 9 and 13, i.e. 1,2-nonanediol and 1,2-tridecanediol, or is selected from the group consisting of 1,2-alkanediols having an uneven number of carbon atoms of 11 and 13, i.e. 1,2-undecanediol and 1,2-tridecanediol.

In a more preferred variant, the second linear alkanediol is selected from the group consisting of 2,3-alkanediols having an uneven number of carbon atoms of 5 to 13, i.e. 2,3-pentanediol, 2,3-heptanediol, 2,3-nonanediol, 2,3-undecanediol, 2,3-tridecanediol and mixtures thereof.

In a still more preferred variant, the second linear alkanediol is selected from the group consisting of 2,3-alkanediols having an uneven number of carbon atoms of 7, 9, 11 and 13, i.e. 2,3-heptanediol, 2,3-nonanediol, 2,3-undecanediol and 2,3-tridecanediol, or is selected from the group consisting of 2,3-alkanediols having an uneven number of carbon atoms of 9 and 11, i.e. 2,3-nonanediol and 2,3-undecanediol, or is selected from the group consisting of 2,3-alkanediols having an uneven number of carbon atoms of 9 and 13, i.e. 2,3-nonanediol and 2,3-tridecanediol, or is selected from the group consisting of 2,3-alkanediols having an uneven number of carbon atoms of 11 and 13, i.e. 2,3-undecanediol and 2,3-tridecanediol.

In a more preferred variant of the cosmetic or pharmaceutical composition according to the second aspect of the present invention, the second linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol and mixtures thereof.

More preferred, in the cosmetic or pharmaceutical composition according to the second aspect of the present invention, the second linear alkanediol is selected from the group consisting of 1,2-pentanediol, 2,3-pentanediol, 3,4-pentanediol, and mixtures thereof. However, the second linear alkanediol can also preferably be selected from the group consisting of 1,2-hexanediol, 2,3-hexanediol, 3,4-hexanediol, and mixtures thereof or can also preferably be selected from the group consisting of 1,2-heptanediol, 2,3-heptanediol, 3,4-heptanediol and mixtures thereof.

Even more preferred, in the cosmetic or pharmaceutical composition according to the second aspect of the present invention, the second linear alkanediol is an alpha,beta or a beta,gamma diol as either 1,2-pentanediol, 2,3-pentanediol or a mixture thereof. However, the second linear alkanediol can also preferably be 1,2-hexanediol or 2,3-hexanediol or a mixture thereof. A mixture comprising 1,2-pentanediol and 2,3-hexanediol or a mixture comprising 2,3-pentanediol and 1,2-hexanediol is also possible.

Most preferred, the second linear alkanediol is 1,2-pentanediol or 2,3-pentanediol or 1,2-hexanediol or 2,3-hexanediol or 1,2-heptanediol or 2,3-heptanediol or 2,3-octanediol or 2,3-nonanediol.

In a particularly preferred variant, the cosmetic or pharmaceutical composition according to the second aspect of the present invention comprises a homo alkanediol mixture or a combination comprising at least one first linear alkanediol and one or more second linear alkanediol includes any one of the following mixtures/combinations:
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-octanediol and 2,3-octanediol;
a mixture comprising 1,2-nonanediol and 2,3-nonanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol; or
a mixture comprising 1,2-tridecanediol and 2,3-tridecanediol.

In said homo alkanediol mixtures the at least first linear alkanediol and the one or more second linear alkanediol have the same number of carbon atoms.

Particularly favourable is a mixture including 1,2-heptanediol in combination with 2,3-heptanediol and/or 3,4-heptanediol, or a mixture including 1,2-octanediol in combination with 2,3-octanediol and/or 3,4-octanediol.

The above specified homo alkanediol mixtures according to the second aspect of the present invention, comprising a 1,2-alkanediol and a 2,3-alkanediol either having the same number of carbon atoms, are characterized in that they improve at least two parameters “spreading”, “fatty/greasy skin feel” and “absorption”, as defined above, of a liquid lipophilic component compared to their respective single 1,2-alkanediol or 2,3-alkanediol substances, as it is demonstrated in the following examples.

Said effect or action in improving the sensory properties of a liquid lipophilic substance in terms of “spreading”, “fatty/greasy skin feel” and “absorption (remaining residue)” is found for the homo mixture including 1,2-pentanediol and 2,3-pentanediol.

Such a sensory improving effect in at least two terms “spreading”, “fatty/greasy skin feel” and “absorption (remaining residue)” is also observed for the homo mixture including 1,2-hexanediol and 2,3-hexanediol.

The homo mixture including 1,2-heptanediol and 2,3-heptanediol is particularly beneficial since it has a particular pronounced effect in improving at least two parameters of the sensory properties of a liquid lipophilic component in a cosmetic or pharmaceutical preparation product.

The alkanediol mixture comprising 1,2-octanediol and 2,3-octanediol or an alkanediol mixture comprising 1,2-nonanediol and 2,3-nonanediol are particularly advantageous, since these mixtures are effective in improving the spreading, or reducing the fatty/greasy skin feel and enhance absorption of a liquid lipophilic component in a cosmetic or pharmaceutical preparation.

Also, the alkanediol mixture comprising 1,2-decanediol and 2,3-decanediol or the alkanediol mixture comprising 1,2-undecanediol and 2,3-undecanediol contributes to improve the spreading, to reduce the fatty/greasy skin feel or to enhance the absorption of a liquid lipophilic component in a cosmetic or pharmaceutical preparation.

Such an improved skin sensation effect is also true for an alkanediol mixture including 1,2-dodecanediol and 2,3-dodecanediol or an alkanediol mixture including 1,2-tridecanediol and 2,3-tridecanediol.

The afore-mentioned intensified effect is particularly pronounced for the homo mixture including 1,2-heptanediol and 2,3-heptanediol or the homo mixture including 1,2-octanediol and 2,3-octanediol or the homo mixture including 1,2-nonanediol and 2,3-nonanediol compared to the corresponding individual 1,2-alkanediol or 2,3-alkanediol substances.

With the afore specified homo alkanediol mixtures comprising a 1,2-alkanediol and the corresponding 2,3-alkanediol it is possible to improve the overall sensory properties characterized by the parameters “spreading”, “fatty/greasy skin feel” and “absorption (remaining residue)” of a liquid lipophilic component, which makes the application in a final formulation such as a cosmetic or pharmaceutical preparation pleasant.

Additionally, with the admixture of the corresponding liquid 2,3-alkanediol to a solid 1,2-alkanediol, the solid 1,2-alkanedioles, such as 1,2-octanediol, 1,2-nonanediol etc., which alone tends to precipitate or recrystallize in oily solutions, can be solved, resulting in a liquid alkanediol mixture. The 2,3-alkanediol serves as solvent for the solid 1,2-alkanediols. Such liquid mixtures have the benefit that firstly the availability of the solid 1,2-alkanediol in the mixture is improved and secondly the incorporation of the solid 1,2-alkanediol in semi-finished products or final products is facilitated.

With the solution of the solid 1,2-octanediol in the liquid 2,3-octanediol or of the solid 1,2-nonanediol in the liquid 2,3-nonanediol, the availability of the 1,2-octanediol or 1,2-nonanediol can be likewise improved in the end use.

In addition, the above homo alkanediol mixtures comprising a 1,2-alkanediol and the corresponding 2,3-alkanediol render possible the cold preparation of cosmetic or pharmaceutical formulations, i.e. no heating during preparation is necessary. Thus, with the afore-mentioned properties of the specified alkanediol mixtures including a 1,2-alkanediol and the respective 2,3-alkanediol the processability in formulations can be improved.

Hence, a good compromise can be achieved when the active mixture is combined in such a way as to improving the sensory effect, while improving processability in formulations. The above specified 1,2-alkanediol and corresponding 2,3-alkanediol combinations solve this balancing problem over the 1,2-alkanediol substances or 2,3-alkanediol substances alone. This is well achieved by combinations such as comprising 1,2-pentanediol and 2,3-pentanediol but also for 1,2-hexanediol and 2,3-hexandiol. Also, combinations such as comprising 1,2-heptanediol and 2,3-heptanediol, but also 1,2-octanediol and 2,3-octanediol show this effect. The same also counts for 1,2-nonanediol in combination with 2,3-nonanediol, but also for 1,2-decanediol in combination with 2,3-decanediol.

The afore specified homo alkanediol mixtures including a 1,2-alkanediol and the corresponding 2,3-alkanediol display a remarkably activity in improving the sensory properties of liquid lipophilic components and are superior to the individually corresponding 1,2-alkanediols or 2,3-alkanediols and having the same concentration.

In a particularly preferred variant, the cosmetic or pharmaceutical composition according to the second aspect of the present invention which comprises a mixture or a combination of the at least one first linear alkanediol with one or more second linear alkanediol thus includes any one of the following combinations:
1,2-pentanediol in combination with one of 1,2-hexanediol, 1,2-heptanediol, 1,2-octanediol, 1,2-nonanediol, 1,2-decanediol, 1,2-undecanediol, 1,2-dodecanediol, or 1,2-tridecanediol;
1,2-hexanediol in combination with one of 1,2-pentanediol, 1,2-heptanediol, 1,2-octanediol, 1,2-nonanediol, 1,2-decanediol, 1,2-undecanediol, 1,2-doedecanediol, or 1,2-tridecanediol;
1,2-heptanediol in combination with one of 1,2-pentanediol, 1,2-hexanediol, 1,2-octanediol, 1,2-nonanediol, 1,2-decanediol, 1,2-undecanediol, 1,2-doedecanediol, or 1,2-tridecanediol;
1,2-octanediol in combination with one of 1,2-pentanediol, 1,2-hexanediol, 1,2-heptanediol, 1,2-nonanediol, 1,2-decanediol, 1,2-undecanediol, 1,2-doedecanediol, or 1,2-tridecanediol;
1,2-nonanediol in combination with one of 1,2-pentanediol, 1,2-hexanediol, 1,2-heptanediol, 1,2-octanediol, 1,2-decanediol, 1,2-undecanediol, 1,2-doedecanediol, or 1,2-tridecanediol;
1,2-decanediol in combination with one of 1,2-pentanediol, 1,2-hexanediol, 1,2-heptanediol, 1,2-octanediol, 1,2-nonanediol, 1,2-undecanediol, 1,2-doedecanediol, or 1,2-tridecanediol;
1,2-undecanediol in combination with one of 1,2-pentanediol, 1,2-hexanediol, 1,2-heptanediol, 1,2-octanediol, 1,2-nonanediol, 1,2-decanediol, 1,2-dodecanediol, or 1,2-tridecanediol;
1,2-dodecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-tridecanediol;
1,2-tridecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-doedecanediol;
2,3-pentanediol in combination with one of 1,2-hexanediol, 1,2-heptanediol, 1,2-octanediol, 1,2-nonanediol, 1,2-decanediol, 1,2-undecanediol, 1,2-doedecanediol, or 1,2-tridecanediol;
2,3-hexanediol in combination with one of 1,2-pentanediol, 1,2-heptanediol, 1,2-octanediol, 1,2-nonanediol, 1,2-decanediol, 1,2-undecanediol, 1,2-doedecanediol, or 1,2-tridecanediol;
2,3-heptanediol in combination with one of 1,2-pentanediol, 1,2-hexanediol, 1,2-octanediol, 1,2-nonanediol, 1,2-decanediol, 1,2-undecanediol, 1,2-doedecanediol, or 1,2-tridecanediol;
2,3-octanediol in combination with one of 1,2-pentanediol, 1,2-hexanediol, 1,2-heptanediol, 1,2-nonanediol, 1,2-decanediol, 1,2-undecanediol, 1,2-doedecanediol, or 1,2-tridecanediol;
2,3-nonanediol in combination with one of 1,2-pentanediol, 1,2-hexanediol, 1,2-heptanediol, 1,2-octanediol, 1,2-decanediol, 1,2-undecanediol, 1,2-doedecanediol, or 1,2-tridecanediol;
2,3-decanediol in combination with one of 1,2-pentanediol, 1,2-hexanediol, 1,2-heptanediol, 1,2-octanediol, 1,2-nonanediol, 1,2-undecanediol, 1,2-doedecanediol, or 1,2-tridecanediol;
2,3-undecanediol in combination with one of 1,2-pentanediol, 1,2-hexanediol, 1,2-heptanediol, 1,2-octanediol, 1,2-nonanediol, 1,2-decanediol, 1,2-dodecanediol, or 1,2-tridecanediol;
2,3-dodecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-tridecanediol; or
2,3-tridecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-dodecanediol;
2,3-pentanediol in combination with one of 2,3-hexanediol, 2,3-heptanediol, 2,3-octanediol, 2,3-nonanediol, 2,3-decanediol, 2,3-undecanediol, 2,3-dodecanediol, or 2,3-tridecanediol;
2,3-hexanediol in combination with one of 2,3-pentanediol, 2,3-heptanediol, 2,3-octanediol, 2,3-nonanediol, 2,3-decanediol, 2,3-undecanediol, 2,3-dodecanediol, or 2,3-tridecanediol;
2,3-heptanediol in combination with one of 2,3-pentanediol, 2,3-hexanediol, 2,3-octanediol, 2,3-nonanediol, 2,3-decanediol, 2,3-undecanediol, 2,3-dodecanediol, or 2,3-tridecanediol;
2,3-octanediol in combination with one of 2,3-pentanediol, 2,3-hexanediol, 2,3-heptanediol, 2,3-nonanediol, 2,3-decanediol, 2,3-undecanediol, 2,3-dodecanediol, or 2,3-tridecanediol;
2,3-nonanediol in combination with one of 2,3-pentanediol, 2,3-hexanediol, 2,3-heptanediol, 2,3-octanediol, 2,3-decanediol, 2,3-undecanediol, 2,3-dodecanediol, or 2,3-tridecanediol;
2,3-decanediol in combination with one of 2,3-pentanediol, 2,3-hexanediol, 2,3-heptanediol, 2,3-octanediol, 2,3-nonanediol, 2,3-undecanediol, 2,3-dodecanediol, or 2,3-tridecanediol;
2,3-undecanediol in combination with one of 2,3-pentanediol, 2,3-hexanediol, 2,3-heptanediol, 2,3-octanediol, 2,3-nonanediol, 2,3-decanediol, 2,3-dodecanediol, or 2,3-tridecanediol;
2,3-dodecanediol in combination with one of 2,3-pentanediol, 2,3-hexanediol, 2,3-heptanediol, 2,3-octanediol, 2,3-nonanediol, 2,3-decanediol, 2,3-undecanediol, or 2,3-tridecanediol; or
2,3-tridecanediol in combination with one of 2,3-pentanediol, 2,3-hexanediol, 2,3-heptanediol, 2,3-octanediol, 2,3-nonanediol, 2,3-decanediol, 2,3-undecanediol, or 2,3-dodecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following mixtures/combinations:
a mixture comprising 1,2-heptanediol and 1,2-pentanediol; or
a mixture comprising 1,2-heptanediol and 1,2-hexanediol; or
a mixture comprising 1,2-heptanediol and 1,2-octanediol; or
a mixture comprising 1,2-heptanediol and 1,2-nonanediol; or
a mixture comprising 1,2-heptanediol and 1,2-decanediol; or
a mixture comprising 1,2-heptanediol and 1,2-undecanediol; or
a mixture comprising 1,2-heptanediol and 1,2-dodecanediol; or
a mixture comprising 1,2-heptanediol and 1,2-tridecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following mixtures/combinations:
a mixture comprising 1,2-heptanediol and 2,3-heptanediol; or
a mixture comprising 1,2-heptanediol and 2,3-pentanediol; or
a mixture comprising 1,2-heptanediol and 2,3-hexanediol; or
a mixture comprising 1,2-heptanediol and 2,3-nonanediol; or
a mixture comprising 1,2-heptanediol and 2,3-decanediol; or
a mixture comprising 1,2-heptanediol and 2,3-undecanediol; or
a mixture comprising 1,2-heptanediol and 2,3-dodecanediol; or
a mixture comprising 1,2-heptanediol and 2,3-tridecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following mixtures/combinations:
1,2-pentanediol and 2,3-pentanediol;
1,2-pentanediol and 2,3-hexanediol;
1,2-pentanediol and 2,3-heptanediol;
1,2-pentanediol and 2,3-octanediol;
1,2-pentanediol and 2,3-nonanediol;
1,2-pentanediol and 2,3-decanediol;
1,2-pentanediol and 2,3-undecanediol;
1,2-pentanediol and 2,3-dodecanediol;
1,2-pentanediol and 2,3-tridecanediol;
1,2-hexanediol and 2,3-pentanediol;
1,2-hexanediol and 2,3-hexanediol;
1,2-hexanediol and 2,3-heptanediol;
1,2-hexanediol and 2,3-octanediol;
1,2-hexanediol and 2,3-nonanediol;
1,2-hexanediol and 2,3-decanediol;
1,2-hexanediol and 2,3-undecanediol;
1,2-hexanediol and 2,3-dodecanediol;
1,2-hexanediol and 2,3-tridecanediol;
1,2-heptanediol and 2,3-pentanediol;
1,2-heptanediol and 2,3-hexanediol;
1,2-heptanediol and 2,3-heptanediol;
1,2-heptanediol and 2,3-octanediol;
1,2-heptanediol and 2,3-nonanediol;
1,2-heptanediol and 2,3-decanediol;
1,2-heptanediol and 2,3-undecanediol;
1,2-heptanediol and 2,3-dodecanediol;
1,2-heptanediol and 2,3-tridecanediol;
1,2-octanediol and 2,3-pentanediol;
1,2-octanediol and 2,3-hexanediol;
1,2-octanediol and 2,3-heptanediol;
1,2-octanediol and 2,3-octanediol;
1,2-octanediol and 2,3-nonanediol;
1,2-octanediol and 2,3-decanediol;
1,2-octanediol and 2,3-undecanediol;
1,2-octanediol and 2,3-dodecanediol;
1,2-octanediol and 2,3-tridecanediol;
1,2-nonanediol and 2,3-pentanediol;
1,2-nonanediol and 2,3-hexanediol;
1,2-nonanediol and 2,3-heptanediol;
1,2-nonanediol and 2,3-octanediol;
1,2-nonanediol and 2,3-nonanediol;
1,2-nonanediol and 2,3-decanediol;
1,2-nonanediol and 2,3-undecanediol;
1,2-nonanediol and 2,3-dodecanediol;
1,2-nonanediol and 2,3-tridecanediol;
1,2-decanediol and 2,3-pentanediol;
1,2-decanediol and 2,3-hexanediol;
1,2-decanediol and 2,3-heptanediol;
1,2-decanediol and 2,3-octanediol;
1,2-decanediol and 2,3-nonanediol;
1,2-decanediol and 2,3-decanediol;
1,2-decanediol and 2,3-undecanediol;
1,2-decanediol and 2,3-dodecanediol;
1,2-decanediol and 2,3-tridecanediol;
1,2-undecanediol and 2,3-pentanediol;
1,2-undecanediol and 2,3-hexanediol;
1,2-undecanediol and 2,3-heptanediol;
1,2-undecanediol and 2,3-octanediol;
1,2-undecanediol and 2,3-nonanediol;
1,2-undecanediol and 2,3-decanediol;
1,2-undecanediol and 2,3-undecanediol;
1,2-undecanediol and 2,3-dodecanediol;
1,2-undecanediol and 2,3-tridecanediol; or
1,2-dodecanediol and 2,3-pentanediol;
1,2-dodecanediol and 2,3-hexanediol;
1,2-dodecanediol and 2,3-heptanediol;
1,2-dodecanediol and 2,3-octanediol;
1,2-dodecanediol and 2,3-nonanediol;
1,2-dodecanediol and 2,3-decanediol;
1,2-dodecanediol and 2,3-undecanediol;
1,2-dodecanediol and 2,3-dodecanediol;
1,2-dodecanediol and 2,3-tridecanediol.

In a particular preferred variant, the cosmetic or pharmaceutical composition according to the second aspect of the present invention which comprises a mixture or a combination of the at least one first linear alkanediol with one or more second linear alkanediol thus may include any of the following mixtures/combinations:
a mixture comprising 2,3-pentanediol and 2,3-hexanediol;
a mixture comprising 2,3-pentanediol and 2,3-heptanediol;
a mixture comprising 2,3-pentanediol and 2,3-octanediol;
a mixture comprising 2,3-pentanediol and 2,3-nonanediol;
a mixture comprising 2,3-pentanediol and 2,3-decanediol;
a mixture comprising 2,3-pentanediol and 2,3-undecanediol;
a mixture comprising 2,3-pentanediol and 2,3-dodecanediol;
a mixture comprising 2,3-pentanediol and 2,3-tridecanediol;
a mixture comprising 2,3-hexanediol and 2,3-pentanediol;
a mixture comprising 2,3-hexanediol and 2,3-heptanediol;
a mixture comprising 2,3-hexanediol and 2,3-octanediol;
a mixture comprising 2,3-hexanediol and 2,3-nonanediol;
a mixture comprising 2,3-hexanediol and 2,3-decanediol;
a mixture comprising 2,3-hexanediol and 2,3-undecanediol;
a mixture comprising 2,3-hexanediol and 2,3-dodecanediol;
a mixture comprising 2,3-hexanediol and 2,3-tridecanediol;
a mixture comprising 2,3-heptanediol and 2,3-pentanediol;
a mixture comprising 2,3-heptanediol and 2,3-hexanediol;
a mixture comprising 2,3-heptanediol and 2,3-octanediol;
a mixture comprising 2,3-heptanediol and 2,3-nonanediol;
a mixture comprising 2,3-heptanediol and 2,3-decanediol;
a mixture comprising 2,3-heptanediol and 2,3-undecanediol;
a mixture comprising 2,3-heptanediol and 2,3-dodecanediol;
a mixture comprising 2,3-heptanediol and 2,3-tridecanediol;
a mixture comprising 2,3-octanediol and 2,3-pentanediol;
a mixture comprising 2,3-octanediol and 2,3-hexanediol;
a mixture comprising 2,3-octanediol and 2,3-heptanediol;
a mixture comprising 2,3-octanediol and 2,3-nonanediol;
a mixture comprising 2,3-octanediol and 2,3-decanediol;
a mixture comprising 2,3-octanediol and 2,3-undecanediol;
a mixture comprising 2,3-octanediol and 2,3-dodecanediol;
a mixture comprising 2,3-octanediol and 2,3-tridecanediol;
a mixture comprising 2,3-nonanediol and 2,3-pentanediol;
a mixture comprising 2,3-nonanediol and 2,3-hexanediol;
a mixture comprising 2,3-nonanediol and 2,3-heptanediol;
a mixture comprising 2,3-nonanediol and 2,3-octanediol;
a mixture comprising 2,3-nonanediol and 2,3-decanediol;
a mixture comprising 2,3-nonanediol and 2,3-undecanediol;
a mixture comprising 2,3-nonanediol and 2,3-dodecanediol;
a mixture comprising 2,3-nonanediol and 2,3-tridecanediol;
a mixture comprising 2,3-decanediol and 2,3-pentanediol;
a mixture comprising 2,3-decanediol and 2,3-hexanediol;
a mixture comprising 2,3-decanediol and 2,3-heptanediol;
a mixture comprising 2,3-decanediol and 2,3-octanediol;
a mixture comprising 2,3-decanediol and 2,3-nonanediol;
a mixture comprising 2,3-decanediol and 2,3-undecanediol;
a mixture comprising 2,3-decanediol and 2,3-dodecanediol;
a mixture comprising 2,3-decanediol and 2,3-tridecanediol;
a mixture comprising 2,3-undecanediol and 2,3-pentanediol;
a mixture comprising 2,3-undecanediol and 2,3-hexanediol;
a mixture comprising 2,3-undecanediol and 2,3-heptanediol;
a mixture comprising 2,3-undecanediol and 2,3-octanediol;
a mixture comprising 2,3-undecanediol and 2,3-nonanediol;
a mixture comprising 2,3-undecanediol and 2,3-decanediol;
a mixture comprising 2,3-undecanediol and 2,3-dodecanediol;
a mixture comprising 2,3-undecanediol and 2,3-tridecanediol;
a mixture comprising 2,3-dodecanediol and 2,3-pentanediol;
a mixture comprising 2,3-dodecanediol and 2,3-hexanediol;
a mixture comprising 2,3-dodecanediol and 2,3-heptanediol;
a mixture comprising 2,3-dodecanediol and 2,3-octanediol;
a mixture comprising 2,3-dodecanediol and 2,3-nonanediol;
a mixture comprising 2,3-dodecanediol and 2,3-decanediol;
a mixture comprising 2,3-dodecanediol and 2,3-dodecanediol;
a mixture comprising 2,3-dodecanediol and 2,3-tridecanediol.

Particularly favourable is a mixture or a combination including 2,3-hexanediol and 2,3-heptanediol or a mixture including 2,3-hexanediol and 2,3-octanediol or a mixture including 2,3-heptanediol and 2,3-octanediol.

In hetero alkanediol mixtures described before the at least first linear alkanediol and the one or more second linear alkanediol have different number of carbon atoms.

The above specified alkanediol mixtures according to the second aspect of the present invention, comprising a first linear alkanediol and a second linear alkanediol, are characterized in that they improve at least two parameters “spreading”, “fatty/greasy skin feel” and “absorption” as described before of a liquid lipophilic component compared to their respective single 1,2-alkanediol or 2,3-alkanediol substances as it is demonstrated in the following examples.

Of the above specified alkanediol mixtures or combinations of the at least one first linear alkanediol with one or more second linear alkanediol particularly favorable is a hetero alkanediol mixture comprising 1,2-hexanediol and 2,3-octanediol or a mixture of alkanediols comprising 1,2-octanediol and 2,3-hexanediol or a mixture of alkanediols comprising 1,2-octanediol and 2,3-heptanediol.

These specified hetero alkanediol mixtures according to the second aspect of the present invention, improve the sensory properties of a liquid lipophilic component in terms of “spreading”, “fatty/greasy skin feel” and “absorption” (remaining residue), compared to their corresponding single 1,2-alkanediol or 2,3-alkanediol substances, namely 1,2- hexanediol or 1,2-octanediol or 2,3-octanediol or 2,3-hexanediol or 2,3-heptanediol.

The hetero alkanediol mixture comprising 1,2-hexanediol and 2,3-octanediol improves the sensory properties as defined above in a cosmetic or pharmaceutical preparation.

Also, the hetero alkanediol mixture comprising 1,2-octanediol and 2,3-hexanediol improves at least two parameters in terms of “spreading”, “fatty/greasy skin feel” and “absorption” (remaining residue) in a cosmetic or pharmaceutical preparation, compared to their corresponding single 1,2-alkanediol or 2,3-alkanediol substances.

An improvement of the sensory properties of a liquid lipophilic component in terms of “spreading”, “fatty/greasy skin feel” and “absorption” (remaining residue) can also be observed for the hetero alkanediol mixture comprising 1,2-octanediol and 2,3-heptanediol.

By the afore specified alkanediol mixtures it is possible to improve the overall sensory properties of a liquid lipophilic component, making the application of a cosmetic or pharmaceutical formulation including this substance more pleasant.

Additionally, with the admixture of 2,3-hexanediol or 2,3-heptanediol to the solid 1,2-octanediol, the solid 1,2-octanediol can be solved, resulting in a liquid alkanediol mixture. The 2,3-hexanediol or the 2,3-heptanediol serves as solvent for the solid 1,2-octanediol. Such liquid mixtures have the benefit that firstly the availability of the 1,2-octanediol in the mixture is improved and secondly the incorporation of the 1,2-octanediol in semi-finished products or final products is facilitated.

Furthermore, an improvement in the sensory properties in terms of “spreading”, “fatty/greasy skin feel” and “absorption” (remaining residue) in a cosmetic or pharmaceutical preparation, can be observed for the following specified alkanediol mixtures or combinations comprising at least one first linear alkanediol and one or more second linear alkanediol according to the second aspect of the present invention, namely a mixture of alkanediols including 1,2-pentanediol and 2,3-hexanediol; or a mixture including 1,2-pentanediol and 1,2-heptanediol; or a mixture including 1,2-pentanediol and 2,3-heptanediol; or a mixture including 1,2-pentanediol and 2,3-octanediol; or a mixture including 1,2-pentanediol and 1,2-nonanediol; or a mixture including 1,2-pentanediol and 2,3-nonanediol.

The afore specified hetero alkane diol mixtures including a first linear alkanediol and a second linear alkanediol display a better spreading and/or a reduced fatty/greasy skin feel and/or an enhanced absorption for a liquid lipophilic component. The intensified action is clearly superior to the corresponding individual 1,2-alkanediols or 2,3-alkanediols.

A hetero alkanediol mixture including 1,2-pentanediol and 2,3-hexanediol remarkably improves the sensory properties of a liquid lipophilic component in a cosmetic or pharmaceutical preparation.

Also, the hetero alkanediol mixture comprising 1,2-pentanediol and 1,2-heptanediol improves at least two parameters “spreading”, “fatty/greasy skin feel” and/or “absorption (remaining residue)” of a liquid lipophilic component in a cosmetic or pharmaceutical preparation.

Such an effect in improving the sensory properties of a liquid lipophilic component can also be observed for the alkanediol mixture comprising 1,2-pentanediol and 2,3-heptanediol.

The spreading, fatty/greasy skin feeling or absorption can also be improved by an alkanediol mixture comprising 1,2-pentanediol and 2,3-octanediol.

Also, an alkanediol mixture comprising 1,2-pentanediol and 1,2-nonanediol shows the effect described above.

Also, the alkanediol mixture including 1,2-pentanediol and 2,3-nonanediol is effective in improving the sensory properties of a liquid lipophilic component.

With the improvement of the sensory properties of a liquid lipophilic component the application of a cosmetic or pharmaceutical preparation including such a substance is more pleasant.

Furthermore, an improvement in sensory properties as described above can also be observed for mixtures comprising at least one first linear alkanediol one or more second linear alkanediol according to the second aspect of the present invention, namely in particular a mixture of alkanediols including 1,2-heptanediol and 1,2-octanediol or a mixture of alkanediols including 1,2-heptanediol and 2,3-octanediol or a mixture of alkanediols including 1,2-heptanediol and 1,2-nonanediol or a mixture or a combination of alkanediols including 1,2-heptanediol and 2,3-nonanediol

In particular, the hetero alkanediol mixture including 1,2-heptanediol and 1,2-octanediol remarkably improves the sensory properties of a liquid lipophilic component, i.e. improved spreading, reduced oily or fatty/greasy skin feel, imparting unwanted gloss to the skin and better absorption, when applied to the skin.

Also, an alkanediol combination including 1,2-heptanediol and 2,3-octanediol show this effect. The same also counts for the alkanediol mixture including 1,2-heptanediol and 2,3-nonanediol.

Consequently, the improvement of the sensory properties of a liquid lipophilic component makes the application of a cosmetic or pharmaceutical formulation comprising such a substance more pleasant.

Also, an alkanediol mixture including 1,2-heptanediol and 1,2-nonanediol or an alkanediol mixture including 1,2-heptanediol and 2,3-nonanediol show an intensification of the activity, i.e. an improvement in at least one parameters “spreading”, “fatty/greasy skin feel” and/or “absorption (remaining residue)” of a liquid lipophilic component in a cosmetic or pharmaceutical preparation.

In a preferred variant of the cosmetic or pharmaceutical composition according to the second aspect of the present invention, the linear alkanediol is selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, and mixtures thereof.

In a more preferred variant of the cosmetic or pharmaceutical composition the linear alkanediol is selected from the group consisting of 2,3-pentanediol, 2,3-hexanediol, 2,3-heptanediol, 2,3-octanediol, 2,3-nonanediol, and mixtures thereof.

Surprisingly, the addition of 2,3-pentanediol, 2,3-hexanediol, 2,3-heptanediol as well as other 2,3-alkanediols such as 2,3-octanediol, 2,3-nonanediol, 2,3-decanediol, 2,3-undecanediol, 2,3-dodecanediol and 2,3-tridecanediol to a liquid lipophilic component as defined herein result in a remarkable improvement in the sensory properties in at least one parameter “spreading”, “fatty/greasy skin feel” and “absorption (remaining residue).

In addition, the 2,3-alkanediols as specified above have the benefit to be liquids, whereby their incorporation in semi-finished products or final products can be facilitated and their availability in said products can be increased. Due to their excellent solubility, the 2,3-alkanediols can be advantageously provided in high concentrations in the composition according to the present invention. This in turn enhances the availability of the 2,3-alkanediols in the topical formulation or composition.

Hence, a good compromise can be achieved when the 2,3-alkanediol is combined in such a way as to maintain the sensory properties effect, while improving processability in formulations. The above specified 2,3-alkanediols solve this balancing problem over the solid 1,2-alkanediol substances.

In a particularly advantageous variant according to the second aspect of the present invention, the linear alkanediol or the first alkanediol or the second alkanediol is a liquid alkanediol.

The term “liquid alkanediol” within the context of the present invention means an alkanediol component which is liquid at room or ambient temperature and under normal pressure, i.e. standard RTP conditions.

In a very preferred variant according to the second aspect of the present invention, the linear alkanediol or the first alkanediol or the second alkanediol is a liquid alkanediol selected from the group consisting of 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, and mixtures thereof.

Of the aforesaid liquid alkanediols, 1,2-pentanediol, 2,3-pentandeiol, 1,2-hexanediol, 2,3-hexanediol, 1,2-heptanediol, 2,3-heptanediol, 2,3-octanediol, 2,3-nonanediol or mixtures thereof are most preferred.

In a most particularly advantageous variant according to the second aspect of the present invention, the linear alkanediol or the first alkanediol or the second alkanediol are characterized by a maximum water solubility of less than or equal to 10% by weight. In a preferred variant, the maximum water solubility of the linear alkanediol or the first alkanediol or the second alkanediol is less than or equal to 10% by weight and more than or equal to 1.2% by weight. More preferred, the maximum water solubility of the linear alkanediol or the first linear alkanediol or the second linear alkanediol is less than or equal to 5% by weight and more than or equal to 1.4% by weight.

Preferably, the alkanediol with the above defined maximum water solubility is selected from the group consisting of 1,2-heptanediol, 2,3-heptanediol, 2,3-octanediol, and mixtures thereof.

The water solubility of a substance is the saturation mass concentration of the substance in water at a given temperature. The determination of the solubility in water relates to essentially pure substances which are stable in water and not volatile. The determination of the water solubility of the alkanediols is further described in the following Example 4.

The term “alkanediol” within the context of the present invention also includes its stereoisomers. Stereoisomers are molecules that have the same molecular formula and differ only in how their atoms are arranged in three-dimensional space. In accordance with said definition, the linear alkanediol of the invention and the first alkanediol having a carbon chain of 5 to 14 carbon atoms or the second linear alkanediol having a carbon chain of 5 to 14 carbon atoms as described above in detail encompass the following stereoisomers:
1,2-alkanediol stereoisomers:
   (2S)-pentane-1,2-diol,
   (2S)-hexane-1,2-diol,
   (2S)-heptane-1,2-diol,
   (2S)-octane-1,2-diol,
   (2S)-nonane-1,2-diol,
   (2S)-decane-1,2-diol,
   (2S)-undecane-1,2-diol
   (2S)-dodecane-1,2-diol,
   (2S)-tridecane-1,2-diol,
   (2S)-tetradecane-1,2-diol,
   (2R)-pentane-1,2-diol,
   (2R)-hexane-1,2-diol,
   (2R)-heptane-1,2-diol,
   (2R)-octane-1,2-diol,
   (2R)-nonane-1,2-diol,
   (2R)-decane-1,2-diol,
   (2R)-undecane-1,2-diol,
   (2R)-dodecane-1,2-diol,
   (2R)-tridecane-1,2-diol,
   (2R)-tetradecane-1,2-diol,
2,3-alkanediol stereoisomers:
   (2S,3S)-pentane-2,3-diol,
   (2S,3S)-hexane-2,3-diol,
   (2S,3S)-heptane-2,3-diol,
   (2S,3S)-octane-2,3-diol,
   (2S, 3S)-nonane-2,3-diol,
   (2S,3S)-decane-2,3-diol,
   (2S,3S)-undecane-2,3-diol
   (2S,3S)-dodecane-2,3-diol,
   (2S,3S)-tridecane-2,3-diol,
   (2S,3S)-tetradecane-2,3-diol,
   (2R,3R)-pentane-2,3-diol,
   (2R, 3R)-hexane-2, 3-diol,
   (2R,3R)-heptane-2,3-diol,
   (2R,3R)-octane-2,3-diol,
   (2R,3R)-nonane-2,3-diol,
   (2R, 3R)-decane-2, 3-diol,
   (2R,3R)-undecane-2,3-diol,
   (2R,3R)-dodecane-2,3-diol,
   (2R,3R)-tridecane-2,3-diol,
   (2R,3R)-tetradecane-2,3-diol,
   (2S,3R)-pentane-2,3-diol,
   (2S, 3R)-hexane-2, 3-diol,
   (2S, 3R)-heptane-2, 3-diol,
   (2S, 3R)-octane-2, 3-diol,
   (2S,3R)-nonane-2,3-diol,
   (2S, 3R)-decane-2,3-diol,
   (2S,3R)-undecane-2,3-diol,
   (2S,3R)-dodecane-2,3-diol,
   (2S, 3R)-tridecane-2,3-diol,
   (2S, 3R)-tetradecane-2,3-diol,
   (2R,3S)-pentane-2,3-diol,
   (2R,3S)-hexane-2,3-diol,
   (2R,3S)-heptane-2,3-diol,
   (2R,3S)-octane-2,3-diol,
   (2R,3S)-nonane-2,3-diol,
   (2R,3S)-decane-2,3-diol,
   (2R,3S)-undecane-2,3-diol,
   (2R,3S)-dodecane-2,3-diol,
   (2R,3S)-tridecane-2,3-diol, and
   (2R,3S)-tetradecane-2,3-diol.
3,4-alkanediol stereoisomers:
   (3R,4R)-hexane-3,4-diol,
   (3R,4R)-heptane-3,4-diol,
   (3R,4R)-octane-3,4-diol,
   (3R,4R)-nonane-3,4-diol,
   (3R,4R)-decane-3,4-diol,
   (3R,4R)-undecane-3,4-diol,
   (3R,4R)-dodecane-3,4-diol,
   (3R,4R)-tridecane-3,4-diol,
   (3R,4R)-tetradecane-3,4-diol,
   (3S,4S)-hexane-3,4-diol,
   (3S,4S)-heptane-3,4-diol,
   (3S,4S)-octane-3,4-diol,
   (3S,4S)-nonane-3,4-diol,
   (3S,4S)-decane-3,4-diol,
   (3S,4S)-undecane-3,4-diol,
   (3S,4S)-dodecane-3,4-diol,
   (3S,4S)-tridecane-3,4-diol,
   (3S,4S)-tetradecane-3,4-diol,
   (3R,4S)-hexane-3,4-diol,
   (3R,4S)-heptane-3,4-diol,
   (3R,4S)-octane-3,4-diol,
   (3R,4S)-nonane-3,4-diol,
   (3R,4S)-decane-3,4-diol,
   (3R,4S)-undecane-3,4-diol,
   (3R,4S)-dodecane-3,4-diol,
   (3R,4S)-tridecane-3,4-diol,
   (3R,4S)-tetradecane-3,4-diol,
   (3S,4R)-hexane-3,4-diol,
   (3S,4R)-heptane-3,4-diol,
   (3S,4R)-octane-3,4-diol,
   (3S,4R)-nonane-3,4-diol,
   (3S,4R)-decane-3,4-diol,
   (3S,4R)-undecane-3,4-diol,
   (3S,4R)-dodecane-3,4-diol,
   (3S,4R)-tridecane-3,4-diol, and
   (3S,4R)-tetradecane-3,4-diol.

In the context of the present text, the terms “1,2-diol”, “2,3-diol” or “3,4-diol” includes both the corresponding S-configured enantiomers and also the R-enantiomeras well as arbitrary mixutres of these S- and R-configured enantiomers, i.e. mixtures of racemates of the respective diols.

The alkanediol mixture of the cosmetic or pharmaceutical composition according to the first aspect of the present invention according to the first aspect of the present invention comprises the 1,2-heptanediol and the 2,3-heptanediol in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90: 10 to 95: 5.

In the cosmetic or pharmaceutical composition according to the first aspect of the present invention, 1,2-heptanediol and 2,3-heptanediol are comprised in the mixture preferably in a ratio in a range of 98 : 2 to 99.9 : 0.1.

1,2-heptanediol and 2,3-heptanediol are comprised in the mixture of the cosmetic or pharmaceutical composition according to the first aspect of the present invention preferred in a ratio of ≥ 95 : ≤ 5, more preferred in a ratio of ≥ 96 : ≤ 4; still more preferred in a ratio of ≥ 97 : ≤ 3, and most preferred in a ratio of ≥ 98 : ≤ 2.

Most of all the mixture of the cosmetic or pharmaceutical composition according to the first aspect of the present invention comprises 1,2-heptanediol and 2,3-heptanediol in a ratio of ≥ 95: ≤ 5, including the ratios ≥ 95.5 : ≤ 4.5; ≥ 96 : ≤ 4; ≥ 96.5 : ≤ 3.5; ≥ 97 : ≤ 3; ≥ 97.5 : 2.5 and ≥ 98.0 : ≤ 2.0.

Even more preferred the alkanediol mixture of the cosmetic or pharmaceutical composition according to the first aspect of the present invention comprises 1,2-heptanediol and 2,3-heptanediol in a ratio of ≥ 98 : ≤ 2, including the ratios of ≥ 98.1 : ≤ 1.9; ≥ 98.2 : ≤ 1.8; ≥ 98.3 : ≤ 1.7; ≥ 98.4 : ≤ 1.6; ≥ 98.5 : ≤ 1.5; ≥ 98.6 : ≤ 1.4; ≥ 98.7 : ≤1.3; ≥ 98.8 : ≤ 1.2; ≥ 98.9 : ≤ 1.1;≥ 99 : ≤ 1.0;≥ 99.1 : ≤ 0.9; ≥ 99.2 : ≤ 0.8; ≥ 99.3 :≤ 0.7; ≥ 99.4 : ≤ 0.6; ≥ 99.5: ≤ 0.5; ≥ 99.6 : ≤ 0.4; ≥ 99.7 : ≤ 0.3; ≥ 99.8 : ≤ 0.2 and ≥ 99.9 : ≤ 0.1.

The first linear alkanediol and the second linear alkanediol are present in the alkanediol mixture according to the second aspect of the present invention in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95: 5.

In the cosmetic or pharmaceutical preparation according to the second aspect of the present invention, the first linear alkanediol and the second linear alkanediol are comprised in the mixture preferably in a ratio in a range of 98 : 2 to 99.9 : 0.1

The first linear alkanediol and the second alkanediol are comprised in the mixture of the cosmetic or pharmaceutical composition according to the second aspect of the present invention preferred in a ratio in a range of ≥ 95 : ≤ 5, more preferred in a ratio of ≥ 96 : ≤ 4; still more preferred in a ratio of ≥ 97 : ≤ 3, and most preferred in a ratio of ≥ 98 : ≤ 2.

Most of all the mixture of the cosmetic or pharmaceutical composition according to the second aspect of the present invention comprises the first linear alkanediol and the second linear alkanediol in a ratio of ≥ 95 : ≤ 5, including the ratios ≥ 95.5 : ≤ 4.5; ≥ 96 : ≤ 4; ≥ 96.5 : ≤ 3.5; ≥ 97 : ≤ 3; ≥ 97.5 : 2.5 and ≥ 98.0 : ≤ 2.0.

Even more preferred the alkanediol mixture of the cosmetic or pharmaceutical composition according to the second aspect of the present invention comprises the first linear alkanediol and the second alkanediol in a ratio ≥ 98 : ≤ 2, including the ratios of ≥ 98.1 : ≤ 1.9; ≥ 98.2 : ≤ 1.8; ≥ 98.3 : ≤ 1.7; ≥ 98.4 : ≤ 1.6; ≥ 98.5 : ≤ 1.5; ≥ 98.6 : ≤ 1.4; ≥ 98.7 : ≤ 1.3; ≥ 98.8 : ≤ 1.2; ≥ 98.9 : ≤ 1.1; ≥ 99 : ≤ 1.0; ≥ 99.1 : ≤ 0.9; ≥ 99.2 : ≤ 0.8; ≥ 99.3 ≤ 0.7; ≥ 99.4 : ≤ 0.6; ≥ 99.5 : ≤ 0.5; ≥ 99.6 : ≤ 0.4; ≥ 99.7 : ≤ 0.3; ≥ 99.8 : ≤ 0.2 and ≥ 99.9 : ≤ 0.1.

Alternatively, said ratio ranges for the first and second alkanediol are switched, such that the second alkanediol is the main component and the first alkanediol is the secondary component.

In an advantageous variant according to the second aspect of the present invention, the mixture comprises as first linear alkanediol an 1,2-alkanediol, such as 1,2-pentanediol, 1,2-hexanediol, 1,2-heptanediol, 1,2-octanediol, 1,2-nonanediol, 1,2-decanediol, 1,2-undecanediol, 1,2-dodecanediol, or 1,2-tridecanediol and as second linear alkanediol the corresponding 2,3-alkanediol, such as 2,3-pentanediol, 2,3-hexanediol, 2,3-heptanediol, 2,3- octanediol, 2,3-nonanediol, 2,3-decanediol, 2,3-undecanediol, 2,3-dodecanediol, or 2,3-tridecanediol in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95 : 5.

Likewise, for said mixtures, the ratios of first linear alkanediol : second alkanediol or ranges of ratios as described above are also applicable.

In a still further variant, according to the second aspect of the present invention, the mixture comprises as first linear alkanediol an 2,3-alkanediol, such as 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol and as second linear alkanediol the corresponding 1,2-alkanediol, such as 1,2-pentanediol, 1,2-hexanediol, 1,2-heptanediol, 1,2- octanediol, 1,2-nonanediol, 1,2-decanediol, 1,2-undecanediol, 1,2-dodecanediol, or 1,2-tridecanediol in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95 : 5.

Likewise, for said mixtures, the ratios of first linear alkanediol : second alkanediol or ranges of ratios as described above are also applicable.

In a more advantageous variant according to the second aspect of the present invention, in the mixtures comprising a combination of a 1,2-alkanediol as first linear alkanediol and the corresponding 2,3-alkanediol as second linear alkanediol, such as
1,2-pentanediol and 2,3-pentanediol, or
1,2-hexanediol and 2,3-hexanediol, or
1,2-heptanediol and 2,3-heptanediol, or
1,2-octanediol and 2,3-octanediol, or
1,2-nonanediol and 2,3-nonanediol, or
1,2-decanediol and 2,2-decanediol, or
1,2-undecanediol and 2,3-undecenaediol, or
1,2-dodecanediol and 2,3-dodecanediol, or
1,2-tridecanediol and 2,3-tridecanediol,
the first linear alkanediol and the second linear alkanediol are present in a ratio in a range of 50 : 50 to 99.9 : 0.1, preferably in a ratio in a range of 75 : 25 to 99 : 1, more preferred in a ratio in a range of 80 : 20 to 98 : 2, still more preferred in ratio in a range of 90 : 10 to 95 : 5.

Likewise, for said mixtures, the ratios of first linear alkanediol : second alkanediol or ranges of ratios as described above are also applicable.

By these mixing ratios the afore mentioned mixtures show an improvement in the sensory properties of a liquid lipophilic component in direct comparison with the corresponding individual substances as it is described and demonstrated in the experimental part.

In a more advantageous variant according to the second aspect of the present invention, in the mixtures comprising as first linear alkanediol a 2,3-alkanediol and as second linear alkanediol the corresponding 1,2-alkanediols, such as mixtures including
2,3-pentanediol and 1,2-pentanediol, or
2,3-hexanediol and 1,2-hexanediol, or
2,3-heptanediol and 1,2-heptanediol, or
2,3-octanediol and 1,2-octanediol, or
2,3-nonanediol and 1,2-nonanediol, or
2,3-decanediol and 1,2-decanediol, or
2,3-undecanediol and 1,2-undecenaediol, or
2,3-dodecanediol and 1,2-dodecanediol, or
2,3-tridecanediol and 1,2-tridecanediol,
the first linear alkanediol and the second linear alkanediol are present in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95 : 5.

Likewise, for said mixtures, the ratios of first linear alkanediol : second alkanediol or ranges of ratios as described above are also applicable.

By these mixing ratios the afore mentioned mixtures show an improvement in the sensory properties of a liquid lipophilic component in direct comparison with the corresponding individual substances as it is described and demonstrated in the experimental part.

A preferred variant according to the second aspect the present invention also encompasses a mixture including as first linear alkanediol 1,2-hexanediol and as second linear alkanediol 2,3-octanediol or a mixture including as first linear alkanediol 1,2-octanediol and as second linear alkanediol 2,3-hexanediol or a mixture including as first linear alkanediol 1,2-octanediol and as second linear alkanediol 2,3-heptanediol either in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95 : 5.

Likewise, for said mixtures, the ratios of first linear alkanediol : second alkanediol or ranges of ratios as described above are also applicable.

By these mixing ratios the afore mentioned mixtures show an improvement in the sensory properties of the liquid component in direct comparison with the corresponding individual substances as it is described and demonstrated in the experimental part.

In a further beneficial variant according to the second aspect of the present invention, the mixtures comprise a first linear alkanediol and a second linear alkanediol, such as mixtures including
1,2-pentanediol and 2,3-hexanediol; or
1,2-pentanediol and 1,2-heptanediol; or
1,2-pentanediol and 2,3-heptanediol; or
1,2-pentanediol and 2,3-octanediol; or
1,2-pentanediol and 1,2-nonanediol; or
1,2-pentanediol and 2,3-nonanediol,
wherein the first linear alkanediol and the second linear alkanediol are present in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95 : 5.

Likewise, for said mixtures, the ratios of first linear alkanediol : second alkanediol or ranges of ratios as described above are also applicable.

By these mixing ratios the afore mentioned mixtures show an improvement in the sensory properties of the liquid component in direct comparison with the corresponding individual substances as it is described and demonstrated in the experimental part.

A particularly preferred variant according to the second aspect the present invention also encompasses a mixture including as first linear alkanediol 1,2-heptanediol and as second linear alkanediol 1,2-octanediol or a mixture including as first linear alkanediol 1,2-heptanediol and as second linear alkanediol 2,3-octanediol or a mixture including as first linear alkanediol 1,2-heptanediol and as second linear alkanediol 1,2-nonanediol or a mixture including as first linear alkanediol 1,2-heptanediol and as second linear alkanediol 2,3-nonanediol wherein the first linear alkanediol and the second linear alkanediol are present in a ratio in a range of 50 : 50 to 99.9 : 0.1, preferably in a ratio in a range of 75 : 25 to 99 : 1, more preferred in a ratio in a range of 80 : 20 to 98 : 2, still more preferred in a ratio in a range of 90 : 10 to 95 : 5.

Likewise, for said mixtures, the ratios of first linear alkanediol : second alkanediol or ranges of ratios as described above are also applicable.

By these mixing ratios the afore mentioned mixtures show an improvement in the sensory properties of the liquid component in direct comparison with the corresponding individual substances as it is described and demonstrated in the experimental part.

The alkanediols are obtained either by synthesis from petrochemical or other fossil fuel sources by known methods such as olefin bishydroxylation, hydrolysis from epoxide or various chemical transformations or from bioderived feedstock by fermentation or from bio-based natural and renewable feedstock such as biomass by catalytic synthesis as it is described in US 2019/0241491 A1 and US 2020/0189995 A1. The alkanediols used according to the present invention comprise either petrochemically derived and biobased natural and renewable feedstock derived alkanediols. Preferably, the alkanediols are from bio-based sources and are thus bio-alkanediols.

The compound (b) of the cosmetic or pharmaceutical composition according to the first aspect or the second aspect of the present invention relates to a liquid lipophilic component, which is used in many cosmetic compositions, in particular skincare, haircare or body care, or for pharmaceutical compositions.

The term “liquid lipophilic component” within the context of the present invention means a lipophilic component which is liquid at room or ambient temperature and under normal pressure, i.e. standard RTP conditions, or at 35 to 37° C. skin temperature; in other words the lipophilic component is liquid at or below 37° C.

The term “lipophilic” (“fat-loving” or “fat-liking”) or “lipophilicity” refers to the ability of a chemical compound to dissolve in fats, oils, lipids and non-polar solvents such as hexane or toluene. Such non-polar solvents are themselves lipophilic. Thus, lipophilic substances tend to dissolve in other lipophilic substances, whereas hydrophilic (“water-loving”) substances tend to dissolve in water and other hydrophilic substances. Therefore, “lipophilic” components are defined according to the present invention in that they are not clearly soluble in water at RTP in a concentration of more than 0.01% by weight. The water solubility of a substance is the saturation mass concentration of the substance in water at a given temperature. The determination of the solubility in water relates to essentially pure substances which are stable in water and not volatile.

The liquid lipophilic component in the cosmetic or pharmaceutical composition according to the present invention encompasses a component selected from the group consisting of liquid plant oils, liquid hydrocarbon, liquid fatty alcohols, liquid fatty acid esters, and liquid UV filters, and mixtures of two or more of the aforesaid liquid oil components.

The liquid lipophilic component in the cosmetic or pharmaceutical composition according to the present invention is preferably a plant oil and even more preferably a liquid plant oil. It can also advantageously be a mixture of two or more plant oils components, especially liquid plant oil mixtures.

Plant oils or vegetable oils are oils extracted from seeds, or less often, from other parts of fruits. Like animal fats, plant oils are mixtures of triglycerides. Soybean oil, rapeseed oil and cocoa butter are examples of plant oils from seeds. Olive oil, palm oil and rice bran oil are examples of oils from other parts of fruits. In common usage, plant oil or vegetable oil may refer exclusively to vegetable fats which are liquid at room temperature or at 35 to 37° C. skin temperature. Vegetable oils are usually edible.

The term “plant oils” also includes unsaturated plant oils. Unsaturated oils or vegetable oils can be transformed through partial or complete “hydrogenation” into oils of higher melting point. The hydrogenation process involves “sparging” the oil at high temperature and pressure with hydrogen in the presence of a catalyst, typically a powdered nickel compound. As each carbon-carbon double-bond is chemically reduced to a single bond, two hydrogen atoms each form single bonds with the two carbon atoms. The elimination of double bonds by adding hydrogen atoms is called saturation; as the degree of saturation increases, the oil progresses toward being fully hydrogenated. An oil may be hydrogenated to increase resistance to rancidity (oxidation) or to change its physical characteristics. As the degree of saturation increases, the oil’s viscosity and melting point increase.

In a preferred variant, the plant oil as the liquid lipophilic component of the cosmetic or pharmaceutical composition of the present invention is selected from the group consisting of Persea Gratissima (Avocado Oil), Abies Alba Seed Oil, Acacia Victoriae Seed Oil, Actinidia Chinensis (Kiwi) Seed Oil, Amaranthus Hypochondriacus Seed Oil, Arachis Hypogaea (Peanut) Oil, Astrocaryum Murumuru Seed Butter, Astrocaryum Tucuma Seed Butter, Astrocaryum Tucuma Seed Oil, Astrocaryum Vulgare Fruit Oil, Astrocaryum Vulgare Kernel Oil, Avena Sativa (Oat) Kernel Oil, Brassica Alba Seed Oil, Brassica Campestris (Rapeseed) Seed Oil, Butyrospermum Parkii (Shea) Butter, Butyrospermum Parkii (Shea) Oil, Calendula Officinalis Seed Oil, Calophyllum Inophyllum Seed Oil, Calophyllum Tacamahaca Seed Oil, Camellia Oleifera Seed Oil, Camellia Reticulata Seed Oil, Camellia Sinensis Seed Oil, Cannabis Sativa Seed Oil, Cannabis Sativa Seed/Stem Oil, Canola Oil, Carthamus Tinctorius (Safflower) Seed Oil, Chlorella Vulgaris Oil, Citrullus Lanatus (Watermelon) Seed Oil, Citrus Aurantifolia (Lime) Seed Oil, Citrus Aurantium Dulcis (Orange) Seed Oil, Citrus Grandis (Grapefruit) Seed Oil, Cocos Nucifera (Coconut) Oil, Cocos Nucifera (Coconut) Seed Butter, Coffea Arabica (Coffee) Seed Oil, Chlorella Oil (biotech), Corylus Americana (Hazelnut) Seed Oil, Corylus Avellana (Hazelnut) Seed Oil, Cucumis Melo (Melon) Seed Oil, Cucumis Sativus (Cucumber) Seed Oil, Cucurbita Pepo (Pumpkin) Seed Oil, Elaeis Guineensis (Palm) Oil, Elaeis (Palm) Fruit Oil, Glycine Soja (Soybean) Oil, Gossypium Herbaceum (Cotton) Seed Oil, Gossypium Hirsutum (Cotton) Seed Oil, Helianthus Annuus (Sunflower) Seed Oil, Macadamia Integrifolia Seed Oil, Macadamia Ternifolia Seed Oil, Mangifera Indica (Mango) Seed Butter, Mangifera Indica (Mango) Seed Oil, Melissa Officinalis Seed Oil, Microalgae Oil, Moringa Oleifera Seed Oil, Moringa Peregrina Seed Oil, Oenothera Biennis (Evening Primrose) Oil, Olea Europaea (Olive) Fruit Oil, Olus Oil, Orbignya Oleifera Seed Oil, Orbignya Speciosa Kernel Oil, Oryza Sativa (Rice) Bran/Germ Oil, Oryza Sativa (Rice) Bran Oil, Oryza Sativa (Rice) Germ Oil, Oryza Sativa (Rice) Lipids, Oryza Sativa (Rice) Seed Oil, Papaver Somniferum Seed Oil, Passiflora Edulis Seed Oil, Persea Gratissima (Avocado) Butter, Persea Gratissima (Avocado) Oil, Prunus Amygdalus Dulcis (Sweet Almond) Oil, Prunus Armeniaca (Apricot) Kernel Oil, Prunus Persica (Peach) Kernel Oil, Punica Granatum Seed Oil, Pyrus Malus (Apple) Seed Oil, Ricinoleic/Caproic/Caprylic/Capric Triglyceride(s), Ricinus Communis (Castor) Seed Oil, Rosa Canina Fruit Oil, Rosa Moschata Seed Oil, Rubus Idaeus (Raspberry) Seed Oil, Sesamum Indicum (Sesame) Seed Butter, Soybean Glycerides, Theobroma Cacao (Cocoa) Seed Butter, Theobroma Grandiflorum Seed Butter, Triticum Vulgare (Wheat) Bran Lipids, Triticum Vulgare (Wheat) Germ Oil, Vitis Vinifera (Grape) Seed Oil, Zea Mays (Corn) Germ Oil, Zea Mays (Corn) Oil, and mixtures of two or more of the aforesaid plant oils.

In a preferred variant, the liquid lipophilic component of the cosmetic or pharmaceutical composition of the present invention is a plant oil selected from the group consisting of Persea Gratissima (Avocado Oil), Arachis Hypogaea (Peanut) Oil, Astrocaryum Murumuru Seed Butter, Avena Sativa (Oat) Kernel Oil, Brassica Campestris (Rapeseed) Seed Oil, Butyrospermum Parkii (Shea) Butter, Butyrospermum Parkii (Shea) Oil, Calendula Officinalis Seed Oil, Calophyllum Inophyllum Seed Oil, Calophyllum Tacamahaca Seed Oil, Camellia Oleifera Seed Oil, Camellia Reticulata Seed Oil, Camellia Sinensis Seed Oil, Cannabis Sativa Seed Oil, Cannabis Sativa Seed/Stem Oil, Canola Oil, Carthamus Tinctorius (Safflower) Seed Oil, Chlorella Vulgaris Oil, Citrus Aurantifolia (Lime) Seed Oil, Citrus Aurantium Dulcis (Orange) Seed Oil, Citrus Grandis (Grapefruit) Seed Oil, Cocos Nucifera (Coconut) Oil, Chlorella Oil (biotech), Elaeis Guineensis (Palm) Oil, Elaeis (Palm) Fruit Oil, Glycine Soja (Soybean) Oil, Gossypium Herbaceum (Cotton) Seed Oil, Gossypium Hirsutum (Cotton) Seed Oil, Helianthus Annuus (Sunflower) Seed Oil, Macadamia Ternifolia Seed Oil, Moringa Oleifera Seed Oil, Oenothera Biennis (Evening Primrose) Oil, Olea Europaea (Olive) Fruit Oil, Olus Oil, Orbignya Oleifera Seed Oil, Orbignya Speciosa Kernel Oil, Oryza Sativa (Rice) Bran/Germ Oil, Oryza Sativa (Rice) Bran Oil, Oryza Sativa (Rice) Germ Oil, Oryza Sativa (Rice) Seed Oil, Papaver Somniferum Seed Oil, Passiflora Edulis Seed Oil, Persea Gratissima (Avocado) Oil, Prunus Amygdalus Dulcis (Sweet Almond) Oil, Prunus Armeniaca (Apricot) Kernel Oil, Prunus Persica (Peach) Kernel Oil, Ricinoleic/Caproic/Caprylic/Capric Triglyceride(s), Ricinus Communis (Castor) Seed Oil, Rosa Canina Fruit Oil, Rosa Moschata Seed Oil, Theobroma Cacao (Cocoa) Seed Butter, Triticum Vulgare (Wheat) Bran Lipids, Triticum Vulgare (Wheat) Germ Oil, Zea Mays (Corn) Germ Oil, Zea Mays (Corn) Oil, and mixtures of two or more of the aforesaid plant oils.

In a more preferred variant, the cosmetic or pharmaceutical composition according to the first aspect or the second aspect of the present invention comprises as liquid lipophilic component a plant oil, selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus AmygdalusDulcis (Sweet Almond) Oil, Ricinus Communis (Castor) Seed Oil, Simmondsia Chinensis (Jojoba) Seed oil, Theobroma Cacao (Cocoa) Seed Butter, Vitis Vinifera (Grape) Seed Oil, Caprylic Capric Triglycerides, and mixtures of two or more of the aforesaid plant oils.

In a particular preferred variant, the cosmetic or pharmaceutical composition according to the first aspect or the second aspect of the present invention comprises as liquid lipophilic component a plant oil, selected from the group consisting of Caprylic Capric Triglycerides, Helianthus Annuus (Sunflower) Seed Oil, Simmondsia Chinensis ((Jojoba) Seed Oil, Olea Europaea (Olive) Fruit Oil, Argania spinosa kernel oil (Argan oil), Prunus Amygdalus Dulcis (Sweet Almond) Oil, Persea Gratissima (Avocado Oil), Butyrospermum Parkii (Shea) Butter, Cocos Nucifera (Coconut) Oil, Theobroma Cacao (Cocoa) Seed Butter, and mixtures of two or more of the aforesaid plant oils.

The plant oil can be used either as a single component or in mixture with one or more further different plant oil(s) as specified above.

The preferred specified plant oils are characterized by a melting point range of - 20° C. to + 5° C.: Glycine Soja (Soybean) Oil, Helianthus Annuus (Sunflower) Seed Oil, Olea Europaea (Olive) Fruit Oil, Prunus Amygdalus Dulcis (Sweet Almond) Oil, and Ricinus Communis (Castor) Seed Oil; by a melting point range of +5° C. to +20° C.: Persea Gratissima (Avocado Oil), Argania spinosa kernel oil (Argan oil), and Simmondsia Chinensis (Jojoba) Seed oil; and by a melting point range of + 20° C. to + 40° C.: Butyrospermum Parkii (Shea) Butter (having a typical melting point in a range of 31° C. to 38° C.), Cocos Nucifera (Coconut) Oil (having a typical melting point at 21-25° C., and Theobroma Cacao (Cocoa) Seed Butter (having a typical melting point in a range of 34° C. to 38° C.).

Preferred plant oils according to the present invention are liquid plant oils with a melting point in the range of - 20° C. to 15° C. and/or 15° C. to 40° C.

In a still more preferred variant according to the present invention, the plant oils have a melting point in the range of - 20° C. to 35° C.

Most preferred are plant oils having a melting point in the range of - 20° C. to ≤ 30° C. These plant oils having a melting point in the range of - 20° C. to ≤ 30° C. are good processible, whereas plant oils having a melting point more than 30° C. have a poor processability.

The plant oils having a melting point in the range of - 20° C. to ≤ 30° C. do not encompass Butyrospermum Parkii (Shea) Butter, having a typical melting point in a range of 31° C. to 38° C.), and Theobroma Cacao (Cocoa) Seed Butter (having a typical melting point in a range of 34° C. to 38° C.).

The plant oils having a melting point in the range of - 20° C. to ≤ 30° C. are selected from the group consisting of Caprylic Capric Triglycerides, Helianthus Annuus (Sunflower) Seed Oil, Simmondsia Chinensis ((Jojoba) Seed Oil, Olea Europaea (Olive) Fruit Oil, Argania spinosa kernel oil (Argan oil), Prunus Amygdalus Dulcis (Sweet Almond) Oil, Persea Gratissima (Avocado Oil), Cocos Nucifera (Coconut) Oil,, and mixtures of two or more of the aforesaid plant oils.

The above specified plant oils are the most common natural lipid components used as basic substances for the manufacture of cosmetics or pharmaceutical formulations. However, the drawback of said plant oils is that they exhibit unpleasant sensory properties upon topical application such as low spreading, an oily, greasy feeling, imparting unwanted gloss to the skin and rendering the composition sticky. Additionally, they are often not easily absorbed or leave an off feeling of the absorbed product on the skin.

Hydrocarbons (mineral oils) are in general organic compounds consisting entirely of hydrogen and carbon. As defined by IUPAC nomenclature or organic chemistry, the classifications for hydrocarbons are:
1. Saturated hydrocarbons are the simplest of the hydrocarbon species. They are composed entirely of single bonds and are saturated with hydrogen. The formula for acyclic saturated hydrocarbons (i.e., alkanes) is CₙH₂ₙ+₂.The most general form of saturated hydrocarbons is CₙH₂ₙ+₂(1₋ᵣ), where r is the number of rings. Those with exactly one ring are the cycloalkanes. Saturated hydrocarbons are the basis of petroleum fuels and are found as either linear or branched species.
2. Unsaturated hydrocarbons have one or more double or triple bonds between carbon atoms. Those with double bond are called alkenes. Those with one double bond have the formula CₙH₂ₙ (assuming non-cyclic structures). Those containing triple bonds are called alkynes. Those with one triple bond have the formula CₙH₂ₙ-₂.
3. Aromatic hydrocarbons, also known as arenes, are hydrocarbons that have at least one aromatic ring.

Hydrocarbons can be inter alia liquids (e.g. hexane and benzene), waxes or low melting solids (e.g. paraffin wax and naphthalene). The term ‘aliphatic’ refers to non-aromatic hydrocarbons. Saturated aliphatic hydrocarbons are sometimes referred to as “paraffins”. Mineral oils and waxes are mixtures of predominantly saturated hydrocarbons consisting of straight-chain, branched and ring structures with carbon chain lengths greater than C14. Mineral oils and waxes are chemical substances prepared from naturally occurring crude petroleum oil. They mainly consist of mineral oil saturated hydrocarbons (MOSH) and mineral oil aromatic hydrocarbons (MOAH). Hydrocarbons have been used for many decades in skin and lip care cosmetic products due to their excellent skin tolerance as well as their high protecting and cleansing performance and broad viscosity options. In contrast to vegetable oils, mineral oils are non-allergenic since they are highly stable and not susceptible to oxidation or rancidity.

In a preferred variant, the hydrocarbon as the liquid lipophilic component of the cosmetic or pharmaceutical composition of the present invention is selected from the group consisting of undecane, tridecane, mineral oil, petrolatum, squalane, isohexadecane, C7 - C8 isoparaffin, C8 - C9 isoparaffin, C9 - C11 isoparaffin, C9 -C12 isoparaffin, C9 - C13 isoparaffin, C9 - C14 isoparaffin, C9 - C16 isoparaffin, C10 - C11 isoparaffin, C10 - C12 isoparaffin, C10 - C13 isoparaffin, C11 - C12 isoparaffin, C11 - C13 isoparaffin, C11 - C14 isoparaffin, C12 - C14 isoparaffin, C12 - C15 isoparaffin, C12 - C20 isoparaffin, C13 - C14 isoparaffin, C13 - C16 isoparaffin, C14 -C16 isoparaffin, C15 - C19 isoparaffin, C18 - C70 isoparaffin, and mixtures of two or more of the aforesaid hydrocarbons.

In a particular preferred variant, the liquid lipophilic component of the cosmetic or pharmaceutical composition of the present invention is a hydrocarbon selected from the group consisting of hydrocarbons, petrolatum, squalane, isohexadecane, C13 -C14 isoparaffin, and mixtures of two or more of the aforesaid hydrocarbons.

The hydrocarbon can be used either as a single component or in mixture with one or more further different hydrocarbon(s) as specified above.

A fatty alcohol (or long-chain alcohol) is usually a high-molecular-weight, straight-chain primary alcohol, but can also range from as few as 4 to 6 carbons to as many as 22 to 26, derived from natural fats and oils. The precise chain length varies with the source. Some commercially important fatty alcohols are lauryl, stearyl and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. Most fatty alcohols in nature are found as waxes which are esters with fatty acids and fatty alcohols. The traditional sources of fatty alcohols have largely been various vegetable oils and these remain a large-scale feedstock. The alcohols are obtained from the triglycerides (fatty acid triesters), which form the bulk of the oil. The process involves the transesterification of the triglycerides to give methyl esters which are then hydrogenated to give the fatty alcohols. Fatty alcohols are also prepared from petrochemical sources. In the Ziegler process, ethylene is oligomerized using triethylaluminium followed by air oxidation. Alternatively, ethylene can be oligomerized to give mixtures of alkenes, which are subjected to hydroformylation, this process affording odd-numbered aldehyde, which is subsequently hydrogenated. Fatty alcohols are mainly used in the production of detergents and surfactants. They are components also of cosmetic solvents. They find use as co-emulsifiers, emollients and thickeners in cosmetics.

In a preferred variant, the fatty alcohol as the liquid lipophilic component of the cosmetic or pharmaceutical composition of the present invention is selected from the group consisting of phenyl propanol, dimethyl phenylbutanol, hexyldecanol, octyldodecanol, octyldecanol, tridecylalcohol, isostearyl alcohol, phenylisohexanol, phenylpropanol, trimethylbenzenepropanol, isoamylalcohol, isostearyl alcohol, isotridecyl alcohol, and mixtures of two or more of the aforesaid fatty alcohols.

In a particular preferred variant, the liquid lipophilic component of the cosmetic or pharmaceutical composition of the present invention is a fatty alcohol selected from the group consisting of hexyldecanol, octyldodecanol, phenylpropanol, isoamylalcohol, and mixtures of two or more of the aforesaid fatty alcohols.

The fatty alcohol can be used either as a single component or in mixture with one or more further different fatty alcohol(s) as specified above.

A fatty acid ester is a type of ester that results from the combination of a fatty acid with an alcohol. When the alcohol component is glycerol, the fatty acid esters produced can be monoglycerides, diglycerides or triglycerides. Fatty acid esters have a conditioning effect of softening the skin to create a smoothing sensation. They are also added to cosmetics to dissolve high-polarity active ingredients and UV absorbers. Esters of straight-chain fatty acids and lower alcohols are effective for dissolving slightly soluble ingredients for oils with a light touch during application. Isostearic acids and other liquid oils with branched fatty acids and unsaturated fatty acids are commonly used as emollients. Higher fatty acid esters and esters of higher alcohols with relatively high melting points are added to skin creams to adjust the application touch.

In a preferred variant, the fatty acid ester as the liquid lipophilic component of the cosmetic or pharmaceutical composition of the present invention is selected from the group consisting of C12 - C15 Alkyl Benzoate, Capric/Lauric/Myristic/Oleic Triglyceride, Caprylic/Capric Triglyceride, Caprylic/Capric/Lauric Triglyceride, Caprylic/Capric/Linoleic Triglyceride, Caprylic/Capric/Myristic/Stearic Triglyceride, Caprylic/Capric/Palmitic/Stearic Triglyceride, Caprylic/Capric/Stearic Triglyceride, Caprylic/Capric/Succinic Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethyl Succinate, Diethylhexyl 2,6-Naphthalate, Diethylhexyl Carbonate, Dibutyl Adipate, Diisopropyl Adipate, Dipropylheptyl Carbonate, Ethyl Laurate, Ethylhexyl Isononanoate, Ethylhexyl Palmitate, Ethylhexyl Stearate, Glyceryl Caprylate Caprate, Glyceryl Caprylate, Glyceryl Laurate, Glyceryl Triacetyl Hydroxystearate, Glyceryl Triacetyl Ricinoleate, Hexyl Laurate, Isoamyl acetate, Isoamyl Cocoate, Isopropyl Palmitate, Isosorbide Dicaprylate, Isopropyl Myristate, Myristyl Myristate, Oleic/Linoleic Triglyceride, Oleic/Palmitic Triglyceride, Oleic/Palmitic/Lauric/Myristic/Linoleic Triglyceride, Propanediol Caprylate, Ricinoleic/Caproic/Caprylic/ Capric Triglyceride, Oleostearine, Oleyl Erucate, Palmitic/Stearic Triglyceride, Propanediol Dicaprylate Caprate, Propylheptyl Caprylate, Stearyl Heptanoate/Stearyl Caprylate, Triheptanoin, Trihydroxystearin, Triisononanoin, Triisopalmitin, Triisostearin, Trilaurin, Trilinolein, Trilinolenin, Trimyristin, Triolein, Tripalmitin, Tripalmitolein, Tripelargonin, Triricinolein, and Tristearin, and mixtures of two or more of the aforesaid fatty acid esters.

In a particular preferred variant, the liquid lipophilic component of the cosmetic or pharmaceutical composition of the present invention is a fatty acid ester selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, Tristearin, and mixtures of two or more of the aforesaid fatty acid esters.

The fatty acid ester can be used either as a single component or in mixture with one or more further different fatty ester(s) as specified above.

An UV filter is a compound or a mixture of compounds that block or absorb ultraviolet (UV) light. Since excessive UV radiation can cause sunburn, photoaging, and skin cancer, care products such as sunscreen usually include a classification for the specific wavelengths they filter. UV classifications include UVA (320 - 400 nm), UVB (290 - 320 nm) and UVC (200 - 280 nm). UV-absorbing compounds are used not only in sunscreen, but also in other personal care products, such as lipstick, shampoo, hair spray, body wash, toilet soap, and insect repellent. Chemical filters protect against UV radiation by absorbing, reflecting, or scattering. Reflection and scattering are accomplished by inorganic physical UV filters, such as titanium dioxide (TiO₂) and zinc oxide (ZnO). Absorption, mainly of UVB, is done by organic UV filters, which are known as chemical UV filters.

In a preferred variant, the UV filter as the liquid lipophilic component of the cosmetic or pharmaceutical composition of the present invention is selected from the group consisting of Octocrylene, Ethylhexyl Salicylate, Homosalate, Ethylhexyl Methoxycinnamate, Isoamyl p-Methoxycinnamate, Camphor Benzalkonium Methosulfate, Polyarylamidomethyl Benzylidene Camphor, Isooctyl Methoxycionnamate, Ethylhexyl Triazone, Drometrizole Trisiloxane, Diethylhexyl Butamido Triazone, 3-Benzylidene Camphor, Octyl Salicylate, Ethylhexyl Dimethyl PABA, Bis-Ethylhexyloxyphenol Methoxyphenyl Triazine, Polysilicone-15, Diethylamino Hydroxybenzoyl Hexyl benzoate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2- Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, Dimethicodiethyl-benzalmalonate, and mixtures of two or more of the aforesaid UV filters.

In a particular preferred variant the liquid lipophilic component of the cosmetic or pharmaceutical composition of the present invention is a UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2- Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, Dimethicodi-ethylbenzalmalonate, and mixtures of two or more of the aforesaid UV filters.

The UV filter can be used either as a single component or in mixture with one or more further different UV filter (s) as specified above.

In a most preferred variant according to the first or second aspect of the present invention, the cosmetic or pharmaceutical composition according to the present invention comprises as liquid lipophilic component preferably a plant oil having a melting point in the range of - 20° C. to ≤ 30° C.; the plant oils are selected from the group consisting of Caprylic Capric Triglycerides, Helianthus Annuus (Sunflower) Seed Oil, Simmondsia Chinensis ((Jojoba) Seed Oil, Olea Europaea (Olive) Fruit Oil, Argania spinosa kernel oil (Argan oil), Prunus Amygdalus Dulcis (Sweet Almond) Oil, Persea Gratissima (Avocado Oil), Cocos Nucifera (Coconut) Oil, and mixtures of two or more of the aforesaid plant oils.

In a particular advantageously variant, the cosmetic or pharmaceutical composition according to the first aspect or second aspect of the present invention comprises one of the following combinations of components (a) and (b):
1,2-pentanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-hexanediol plus one or more plant oil(s) selected from the group consisting of Persea Gratissima (Avocado Oil), Argania spinosa kernel oil (Argan oil), Butyrospermum Parkii (Shea) Butter, Cocos Nucifera Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-octanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-nonanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-decanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-undecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-dodecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-tridecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-pentanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-hexanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-heptanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-octanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-nonanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-decanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-undecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-dodecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-tridecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with 2,3-heptanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, RicinusCommunis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 2,3-pentanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-hexanediol in combination with 2,3-hexanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-octanediol in combination with 2,3-octanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-nonanediol in combination with 2,3-nonanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-decanediol in combination with 2,3-decanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-undecanediol in combination with 2,3-undecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-dodecanediol in combination with 2,3-dodecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-tridecanediol in combination with 2,3-tridecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-hexanediol in combination with 2,3-octanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-octanediol in combination with 2,3-hexanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-octanediol in combination with 2,3-heptanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 2,3-hexanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 1,2-heptanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 2,3-heptanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 2,3-octanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 1,2-nonanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-pentanediol in combination with 2,3-nonanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with 1,2-octanediol plus one or more plant oil(s), selected from the group consisting of Persea Gratissima Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with 2,3-octanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with 1,2-nonanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with 2,3-nonanediol plus one or more plant oil(s), selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil.

More preferred combinations of component (a) and (b) in the cosmetic or pharmaceutical composition according to the first aspect or second aspect of the present invention are:
1,2-heptanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
2,3-heptanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with 2,3-heptanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol, or 1,2-tridecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil; or
1,2-heptanediol in combination with one or more of 2,3-pentanediol, 2,3-hexanediol, 2,3-octanediol, 2,3-nonanediol, 2,3-decanediol, 2,3-undecanediol, 2,3-dodecanediol or 2,3-tridecanediol plus one or more plant oil(s) selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, and Vitis Vinifera (Grape) Seed Oil.

In a most preferred variant, the cosmetic or pharmaceutical composition according to the first aspect of the present invention comprises the following combinations of components (a) and (b):
1,2-heptanediol plus one or more plant oil(s) selected from the group consisting of Persea Gratissima (Avocado Oil), Argania spinosa kernel oil (Argan oil), Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, and Vitis Vinifera (Grape) Seed Oil.

In a particular advantageously variant, the cosmetic or pharmaceutical composition according to the first aspect or second aspect of the present invention comprises one of the following combinations of components (a) and (b):
1,2-pentanediol plus one or more hydrocarbon(s) selected from the group consisting of hydrocarbons, petrolatum, squalane, isohexadecane, and C13 -C14 isoparaffin; or
1,2-hexanediol plus one or more hydrocarbon(s) selected from the group consisting of hydrocarbons, petrolatum, squalane, isohexadecane, and C13 -C14 isoparaffin; or
1,2-heptanediol plus one or more hydrocarbon(s) selected from the group consisting of hydrocarbons, petrolatum, squalane, isohexadecane, and C13 -C14 isoparaffin; or
1,2-octanediol plus one or more hydrocarbon(s) selected from the group consisting of hydrocarbons, petrolatum, squalane, isohexadecane, and C13 -C14 isoparaffin; or
1,2-nonanediol plus one or more hydrocarbon(s) selected from the group consisting of hydrocarbons, petrolatum, squalane, isohexadecane, and C13 -C14 isoparaffin;or
1,2-decanediol plus one or more hydrocarbon(s) selected from the group consisting of hydrocarbons, petrolatum, squalane, isohexadecane, C13 - C14 isoparaffin, and mixtures of two or more of the aforesaid hydrocarbons; or
1,2-undecanediol plus one or more hydrocarbon(s) selected from the group consisting of hydrocarbons, petrolatum, squalane, isohexadecane, and C13 -C14 isoparaffin; or
1,2-dodecanediol plus one or more hydrocarbon(s) selected from the group consisting of hydrocarbons, petrolatum, squalane, isohexadecane, and C13 -C14 isoparaffin; or
1,2-tridecanediol plus one or more hydrocarbon(s) selected from the group consisting of hydrocarbons, petrolatum, squalane, isohexadecane, and C13 -C14 isoparaffin; or
2,3-pentanediol plus one or more hydrocarbon(s) selected from the group consisting of hydrocarbons, petrolatum, squalane, isohexadecane, and C13 -C14 isoparaffin; or
2,3-hexanediol plus one or more hydrocarbon(s) selected from the group consisting of hydrocarbons, petrolatum, squalane, isohexadecane, and C13 -C14 isoparaffin; or
2,3-heptanediol plus one or more hydrocarbon(s) selected from the group consisting of hydrocarbons, petrolatum, squalane, isohexadecane, and C13 -C14 isoparaffin; or
2,3-octanediol plus one or more hydrocarbon(s) selected from the group consisting of hydrocarbons, petrolatum, squalane, isohexadecane, and C13 -C14 isoparaffin; or
2,3-nonanediol plus one or more hydrocarbon(s) selected from the group consisting of hydrocarbons, petrolatum, squalane, isohexadecane, and C13 -C14 isoparaffin; or
2,3-decanediol plus one or more hydrocarbon(s) selected from the group consisting of hydrocarbons, petrolatum, squalane, isohexadecane, and C13 -C14 isoparaffin; or
2,3-undecanediol plus one or more hydrocarbon(s) selected from the group consisting of hydrocarbons, petrolatum, squalane, isohexadecane, and C13 -C14 isoparaffin; or
2,3-dodecanediol plus one or more hydrocarbon(s) selected from the group consisting of hydrocarbons, petrolatum, squalane, isohexadecane, and C13 -C14 isoparaffin; or
2,3-tridecanediol plus one or more hydrocarbon(s) selected from the group consisting of hydrocarbons, petrolatum, squalane, isohexadecane, and C13 -C14 isoparaffin; or
1,2-pentanediol in combination with 2,3-pentanediol plus one or more hydrocarbon(s) selected from the group consisting of hydrocarbons, petrolatum, squalane, isohexadecane, and C13 - C14 isoparaffin; or
1,2-heptanediol in combination with 2,3-heptanediol plus one or more hydrocarbon(s) selected from the group consisting of hydrocarbons, petrolatum, squalane, isohexadecane, and C13 - C14 isoparaffin; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or ore hydrocarbon(s) selected from the group consisting of hydrocarbons, petrolatum, squalane, isohexadecane, and C13 - C14 isoparaffin; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more hydrocarbon(s) selected from the group consisting of hydrocarbons, petrolatum, squalane, isohexadecane, and C13 - C14 isoparaffin; or
1,2-hexanediol in combination with 2,3-hexanediol plus one or more hydrocarbon(s) selected from the group consisting of hydrocarbons, petrolatum, squalane, isohexadecane, and C13 - C14 isoparaffin; or
1,2-octanediol in combination with 2,3-octanediol plus one or more hydrocarbon(s) selected from the group consisting of hydrocarbons, petrolatum, squalane, isohexadecane, and C13 - C14 isoparaffin; or
1,2-nonanediol in combination with 2,3-nonanediol plus one or more hydrocarbon(s) selected from the group consisting of hydrocarbons, petrolatum, squalane, isohexadecane, and C13 - C14 isoparaffin; or
1,2-decanediol in combination with 2,3-decanediol plus one or more hydrocarbon(s) selected from the group consisting of hydrocarbons, petrolatum, squalane, isohexadecane, and C13 - C14 isoparaffin; or
1,2-undecanediol in combination with 2,3-undecanediol plus one or more hydrocarbon(s) selected from the group consisting of hydrocarbons, petrolatum, squalane, isohexadecane, and C13 - C14 isoparaffin; or
1,2-dodecanediol in combination with 2,3-dodecanediol plus one or more hydrocarbon(s) selected from the group consisting of hydrocarbons, petrolatum, squalane, isohexadecane, and C13 - C14 isoparaffin; or
1,2-tridecanediol in combination with 2,3-tridecanediol plus one or more hydrocarbon(s) selected from the group consisting of hydrocarbons, petrolatum, squalane, isohexadecane, and C13 - C14 isoparaffin; or
1,2-hexanediol in combination with 2,3-octanediol plus one or more hydrocarbon(s) selected from the group consisting of hydrocarbons, petrolatum, squalane, isohexadecane, and C13 - C14 isoparaffin; or
1,2-octanediol in combination with 2,3-hexanediol plus one or more hydrocarbon(s) selected from the group consisting of hydrocarbons, petrolatum, squalane, isohexadecane, and C13 - C14 isoparaffin; or
1,2-octanediol in combination with 2,3-heptanediol plus one or more hydrocarbon(s) selected from the group consisting of hydrocarbons, petrolatum, squalane, isohexadecane, and C13 - C14 isoparaffin; or
1,2-pentanediol in combination with 2,3-hexanediol plus one or more hydrocarbon(s) selected from the group consisting of hydrocarbons, petrolatum, squalane, isohexadecane, and C13 - C14 isoparaffin; or
1,2-pentanediol in combination with 1,2-heptanediol plus one or more hydrocarbon(s) selected from the group consisting of hydrocarbons, petrolatum, squalane, isohexadecane, and C13 - C14 isoparaffin; or
1,2-pentanediol in combination with 2,3-heptanediol plus one or more hydrocarbon(s) selected from the group consisting of hydrocarbons, petrolatum, squalane, isohexadecane, and C13 - C14 isoparaffin; or
1,2-pentanediol in combination with 2,3-octanediol plus one or more hydrocarbon(s) selected from the group consisting of hydrocarbons, petrolatum, squalane, isohexadecane, and C13 - C14 isoparaffin; or
1,2-pentanediol in combination with 1,2-nonanediol plus one or more hydrocarbon(s) selected from the group consisting of hydrocarbons, petrolatum, squalane, isohexadecane, and C13 - C14 isoparaffin; or
1,2-pentanediol in combination with 2,3-nonanediol plus one or more hydrocarbon(s) selected from the group consisting of hydrocarbons, petrolatum, squalane, isohexadecane, and C13 - C14 isoparaffin; or
1,2-heptanediol in combination with 1,2-octanediol plus one or more hydrocarbon(s) selected from the group consisting of hydrocarbons, petrolatum, squalane, isohexadecane, and C13 - C14 isoparaffin; or
1,2-heptanediol in combination with 2,3-octanediol plus one or more hydrocarbon(s) selected from the group consisting of hydrocarbons, petrolatum, squalane, isohexadecane, and C13 - C14 isoparaffin; or
1,2-heptanediol in combination with 1,2-nonanediol plus one or more hydrocarbon(s) selected from the group consisting of hydrocarbons, petrolatum, squalane, isohexadecane, and C13 - C14 isoparaffin; or
1,2-heptanediol in combination with 2,3-nonanediol plus one or more hydrocarbon(s) selected from the group consisting of hydrocarbons, petrolatum, squalane, isohexadecane, and C13 - C14 isoparaffin.

The most preferred combinations of component (a) and (b) in the cosmetic or pharmaceutical composition according to the first aspect or second aspect of the present invention are:
1,2-heptanediol plus one or more hydrocarbon(s) selected from the group consisting of hydrocarbons, petrolatum, squalane, isohexadecane, and C13 -C14 isoparaffin; or
2,3-heptanediol plus one or more hydrocarbon(s) selected from the group consisting of hydrocarbons, petrolatum, squalane, isohexadecane, and C13 -C14 isoparaffin; or
1,2-heptanediol in combination with 2,3-heptanediol plus one or more hydrocarbon(s) selected from the group consisting of hydrocarbons, petrolatum, squalane, isohexadecane, and C13 - C14 isoparaffin; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol, or 1,2-tridecanediol plus one or more hydrocarbon(s) selected from the group consisting of hydrocarbons, petrolatum, squalane, isohexadecane, and C13 - C14 isoparaffin; or
1,2-heptanediol in combination with one or more of 2,3-pentanediol, 2,3-hexanediol, 2,3-octanediol, 2,3-nonanediol, 2,3-decanediol, 2,3-undecanediol, 2,3-dodecanediol or 2,3-tridecanediol plus one or more hydrocarbon(s) selected from the group consisting of hydrocarbons, petrolatum, squalane, isohexadecane, and C13 - C14 isoparaffin.

In a particular advantageously variant, the cosmetic or pharmaceutical composition according to the first aspect or second aspect of the present invention comprises one of the following combinations of components (a) and (b):
1,2-pentanediol plus one or more fatty alcohol(s) selected from the group consisting of hexyldecanol, octyldodecanol, phenylpropanol, and isoamylalcohol; or
1,2-hexanediol plus one or more fatty alcohol(s) selected from the group consisting of hexyldecanol, octyldodecanol, phenylpropanol, and isoamylalcohol; or
1,2-heptanediol plus one or more fatty alcohol(s) selected from the group consisting of hexyldecanol, octyldodecanol, phenylpropanol, and isoamylalcohol; or
1,2-octanediol plus one or more fatty alcohol(s) selected from the group consisting of hexyldecanol, octyldodecanol, phenylpropanol, and isoamylalcohol; or
1,2-nonanediol plus one or more fatty alcohol(s) selected from the group consisting of hexyldecanol, octyldodecanol, phenylpropanol, and isoamylalcohol; or
1,2-decanediol plus one or more fatty alcohol(s) selected from the group consisting of hexyldecanol, octyldodecanol, phenylpropanol, and isoamylalcohol; or
1,2-undecanediol plus one or more fatty alcohol(s) selected from the group consisting of hexyldecanol, octyldodecanol, phenylpropanol, and isoamylalcohol; or
1,2-dodecanediol plus one or more fatty alcohol(s) selected from the group consisting of hexyldecanol, octyldodecanol, phenylpropanol, and isoamylalcohol;
1,2-tridecanediol plus one or more fatty alcohol(s) selected from the group consisting of hexyldecanol, octyldodecanol, phenylpropanol, isoamylalcohol, and mixtures of two or more of the aforesaid fatty alcohols; or
2,3-pentanediol plus one or more fatty alcohol(s) selected from the group consisting of hexyldecanol, octyldodecanol, phenylpropanol, and isoamylalcohol; or
2,3-hexanediol plus one or more fatty alcohol(s) selected from the group consisting of hexyldecanol, octyldodecanol, phenylpropanol, and isoamylalcohol; or
2,3-heptanediol plus one or more fatty alcohol(s) selected from the group consisting of hexyldecanol, octyldodecanol, phenylpropanol, and isoamylalcohol; or
2,3-octanediol plus one or more fatty alcohol(s) selected from the group consisting of hexyldecanol, octyldodecanol, phenylpropanol, and isoamylalcohol; or
2,3-nonanediol plus one or more fatty alcohol(s) selected from the group consisting of hexyldecanol, octyldodecanol, phenylpropanol, and isoamylalcohol; or
2,3-decanediol plus one or more fatty alcohol(s) selected from the group consisting of hexyldecanol, octyldodecanol, phenylpropanol, and isoamylalcohol; or
2,3-undecanediol plus one or more fatty alcohol(s) selected from the group consisting of hexyldecanol, octyldodecanol, phenylpropanol, and isoamylalcohol; or
2,3-dodecanediol plus one or more fatty alcohol(s) selected from the group consisting of hexyldecanol, octyldodecanol, phenylpropanol, and isoamylalcohol; or
2,3-tridecanediol plus one or more fatty alcohol(s) selected from the group consisting of hexyldecanol, octyldodecanol, phenylpropanol, and isoamylalcohol; or
1,2-heptanediol in combination with 2,3-heptanediol plus one or more fatty alcohol(s) selected from the group consisting of hexyldecanol, octyldodecanol, phenylpropanol, and isoamylalcohol; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or more fatty alcohol(s) selected from the group consisting of hexyldecanol, octyldodecanol, phenylpropanol, and isoamylalcohol; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more fatty alcohol(s) selected from the group consisting of hexyldecanol, octyldodecanol, phenylpropanol, and isoamylalcohol; or
1,2-pentanediol in combination with 2,3-pentanediol plus one or more fatty alcohol(s) selected from the group consisting of hexyldecanol, octyldodecanol, phenylpropanol, and isoamylalcohol; or
1,2-hexanediol in combination with 2,3-hexanediol plus one or more fatty alcohol(s) selected from the group consisting of hexyldecanol, octyldodecanol, phenylpropanol, and isoamylalcohol; or
1,2-octanediol in combination with 2,3-octanediol plus one or more fatty alcohol(s) selected from the group consisting of hexyldecanol, octyldodecanol, phenylpropanol, and isoamylalcohol; or
1,2-nonanediol in combination with 2,3-nonanediol plus one or more fatty alcohol(s) selected from the group consisting of hexyldecanol, octyldodecanol, phenylpropanol, and isoamylalcohol; or
1,2-decanediol in combination with 2,3-decanediol plus one or more fatty alcohol(s) selected from the group consisting of hexyldecanol, octyldodecanol, phenylpropanol, and isoamylalcohol; or
1,2-undecanediol in combination with 2,3-undecanediol plus one or more fatty alcohol(s) selected from the group consisting of hexyldecanol, octyldodecanol, phenylpropanol, and isoamylalcohol; or
1,2-dodecanediol in combination with 2,3-dodecanediol plus one or more fatty alcohol(s) selected from the group consisting of hexyldecanol, octyldodecanol, phenylpropanol, and isoamylalcohol; or
1,2-tridecanediol in combination with 2,3-tridecanediol plus one or more fatty alcohol(s) selected from the group consisting of hexyldecanol, octyldodecanol, phenylpropanol, and isoamylalcohol; or
1,2-hexanediol in combination with 2,3-octanediol plus one or more fatty alcohol(s) selected from the group consisting of hexyldecanol, octyldodecanol, phenylpropanol, and isoamylalcohol; or
1,2-octanediol in combination with 2,3-hexanediol plus one or more fatty alcohol(s) selected from the group consisting of hexyldecanol, octyldodecanol, phenylpropanol, and isoamylalcohol; or
1,2-octanediol in combination with 2,3-heptanediol plus one or more fatty alcohol(s) selected from the group consisting of hexyldecanol, octyldodecanol, phenylpropanol, and isoamylalcohol; or
1,2-pentanediol in combination with 2,3-hexanediol plus one or more fatty alcohol(s) selected from the group consisting of hexyldecanol, octyldodecanol, phenylpropanol, and isoamylalcohol; or
1,2-pentanediol in combination with 1,2-heptanediol plus one or more fatty alcohol(s) selected from the group consisting of hexyldecanol, octyldodecanol, phenylpropanol, and isoamylalcohol; or
1,2-pentanediol in combination with 2,3-heptanediol plus one or more fatty alcohol(s) selected from the group consisting of hexyldecanol, octyldodecanol, phenylpropanol, and isoamylalcohol; or
1,2-pentanediol in combination with 2,3-octanediol plus one or more fatty alcohol(s) selected from the group consisting of hexyldecanol, octyldodecanol, phenylpropanol, and isoamylalcohol; or
1,2-pentanediol in combination with 1,2-nonanediol plus one or more fatty alcohol(s) selected from the group consisting of hexyldecanol, octyldodecanol, phenylpropanol, and isoamylalcohol; or
1,2-pentanediol in combination with 2,3-nonanediol plus one or more fatty alcohol(s) selected from the group consisting of hexyldecanol, octyldodecanol, phenylpropanol, and isoamylalcohol; or
1,2-heptanediol in combination with 1,2-octanediol plus one or more fatty alcohol(s) selected from the group consisting of hexyldecanol, octyldodecanol, phenylpropanol, and isoamylalcohol; or
1,2-heptanediol in combination with 2,3-octanediol plus one or more fatty alcohol(s) selected from the group consisting of hexyldecanol, octyldodecanol, phenylpropanol, and isoamylalcohol; or
1,2-heptanediol in combination with 1,2-nonanediol plus one or more fatty alcohol(s) selected from the group consisting of hexyldecanol, octyldodecanol, phenylpropanol, and isoamylalcohol; or
1,2-heptanediol in combination with 2,3-nonanediol plus one or more fatty alcohol(s) selected from the group consisting of hexyldecanol, octyldodecanol, phenylpropanol, and isoamylalcohol.

The most preferred combinations of component (a) and (b) in the cosmetic or pharmaceutical composition according to the first aspect or second aspect of the present invention are:
1,2-heptanediol plus one or more fatty alcohol(s) selected from the group consisting of hexyldecanol, octyldodecanol, phenylpropanol, and isoamylalcohol; or
2,3-heptanediol plus one or more fatty alcohol(s) selected from the group consisting of hexyldecanol, octyldodecanol, phenylpropanol, and isoamylalcohol; or
1,2-heptanediol in combination with 2,3-heptanediol plus one or more fatty alcohol(s) selected from the group consisting of hexyldecanol, octyldodecanol, phenylpropanol, and isoamylalcohol; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol, or 1,2-tridecanediol plus one or more fatty alcohol(s) selected from the group consisting of hexyldecanol, octyldodecanol, phenylpropanol, and isoamylalcohol; or
1,2-heptanediol in combination with one or more of 2,3-pentanediol, 2,3-hexanediol, 2,3-octanediol, 2,3-nonanediol, 2,3-decanediol, 2,3-undecanediol, 2,3-dodecanediol or 2,3-tridecanediol plus one or more fatty alcohol(s) selected from the group consisting of hexyldecanol, octyldodecanol, phenylpropanol, and isoamylalcohol.

In a particular advantageously variant, the cosmetic or pharmaceutical composition according to the first aspect or second aspect of the present invention comprises one of the following combinations of components (a) and (b):
1,2-pentanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-hexanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-decanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-undecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-dodecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-tridecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-pentanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-hexanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-decanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-undecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-dodecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-tridecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with 2,3-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 2,3-pentanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-hexanediol in combination with 2,3-hexanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-octanediol in combination with 2,3-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-nonanediol in combination with 2,3-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-decanediol in combination with 2,3-decanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-undecanediol in combination with 2,3-undecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-dodecanediol in combination with 2,3-dodecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-tridecanediol in combination with 2,3-tridecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-hexanediol in combination with 2,3-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-octanediol in combination with 2,3-hexanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-octanediol in combination with 2,3-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 2,3-hexanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 1,2-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 2,3-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 2,3-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 1,2-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-pentanediol in combination with 2,3-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
■1,2-heptanediol in combination with 1,2-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with 2,3-octanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with 1,2-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with 2,3-nonanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin.

The most preferred combinations of component (a) and (b) in the cosmetic or pharmaceutical composition according to the first aspect or second aspect of the present invention are:
1,2-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
2,3-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with 2,3-heptanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol, or 1,2-tridecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin; or
1,2-heptanediol in combination with one or more of 2,3-pentanediol, 2,3-hexanediol, 2,3-octanediol, 2,3-nonanediol, 2,3-decanediol, 2,3-undecanediol, 2,3-dodecanediol or 2,3-tridecanediol plus one or more fatty acid ester(s) selected from the group consisting of C12 - C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, and Tristearin.

In a particular advantageously variant, the cosmetic or pharmaceutical composition according to the first aspect or second aspect of the present invention comprises one of the following combinations of components (a) and (b):
1,2-pentanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-hexanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-decanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-undecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/Bisoctrizole, and Dimethicodiethylbenzalmalonate; or
1,2-dodecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/Bisoctrizole, and Dimethicodiethylbenzalmalonate; or
1,2-tridecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
2,3-pentanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
2,3-hexanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
2,3-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
2,3-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
2,3-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
2,3-decanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
2,3-undecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/Bisoctrizole, and Dimethicodiethylbenzalmalonate; or
2,3-dodecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/Bisoctrizole, and Dimethicodiethylbenzalmalonate; or
2,3-tridecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with 2,3-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/Bisoctrizole, and Dimethicodiethylbenzalmalonate; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-hexanediol in combination with 2,3-hexanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-octanediol in combination with 2,3-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-nonanediol in combination with 2,3-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-decanediol in combination with 2,3-decanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-undecanediol in combination with 2,3-undecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-dodecanediol in combination with 2,3-dodecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate; or
1,2-tridecanediol in combination with 2,3-tridecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-hexanediol in combination with 2,3-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-octanediol in combination with 2,3-hexanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-octanediol in combination with 2,3-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-pentanediol in combination with 2,3-hexanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-pentanediol in combination with 1,2-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-pentanediol in combination with 2,3-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-pentanediol in combination with 2,3-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-pentanediol in combination with 1,2-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-pentanediol in combination with 2,3-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with 1,2-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with 2,3-octanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with 1,2-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with 2,3-nonanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate.

The most preferred combinations of component (a) and (b) in the cosmetic or pharmaceutical composition according to the first aspect or second aspect of the present invention are:
1,2-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
2,3-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with 2,3-heptanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl- butyl)phenol)/Bisoctrizole, and Dimethicodiethylbenzalmalonate; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol, or 1,2-tridecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/ Bisoctrizole, and Dimethicodiethylbenzalmalonate; or
1,2-heptanediol in combination with one or more of 2,3-pentanediol, 2,3-hexanediol, 2,3-octanediol, 2,3-nonanediol, 2,3-decanediol, 2,3-undecanediol, 2,3-dodecanediol or 2,3-tridecanediol plus one or more UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethylbutyl)phenol)/Bisoctrizole, and Dimethicodiethylbenzalmalonate.

Of the above combinations, the combinations with 1,2-heptanediol or a 2,3-alkanediol, in particular 2,3-pentanediol, 2,3-hexanediol, 2,3-heptanediole, 2,3-octanediole or 2,3-nonanediol, or a mixture including a 1,2-alkanediol and a 2,3-alkanediol as defined above, result in distinguished sensory properties, i.e. skin sensation, upon topical application, i.e. improved spreading behaviour, less fatty/greasy skin feeling and better absorption of the liquid lipophilic component upon application on the skin. These effects are described in more detail below in connection with the examples.

The above-defined specific combinations can be combined with one or more further components (c) as described later on.

Within the context of the present invention, it is also possible and in some cases advantageous to combine the cosmetic or pharmaceutical composition according to the first aspect or second aspect of the present invention with other active substances, adjuvants or additives.

The cosmetically or pharmaceutically active agents and/or adjuvants and/or additives can in some instances provide one or more than one benefit or operate via more than one mode of action.

Thus, optionally, other conventional cosmetically and/or pharmaceutically active substances, adjuvants or additives, as further described below, may be added as component (c), i.e. in order to obtain a ready-for-use composition or formulation.

The cosmetic or pharmaceutical composition according to the present invention can advantageously be combined with other cosmetically or pharmaceutically active agents and/or adjuvants and/or additives or auxiliaries, such as are customarily used in such compositions, such as for example abrasives, anti-acne agents, agents against ageing of the skin, anti-cellulitis agents, anti-dandruff agents, anti-inflammatory agents, anti-microbial agents, irritation-preventing agents, irritation-inhibiting agents, antioxidants, astringents, odor absorbers, perspiration-inhibiting agents, antiseptic agents, anti-statics, binders, buffers, carrier materials, chelating agents, cell stimulants, cleansing agents, depilatory agents, surface-active substances, deodorizing agents, antiperspirants, softeners, emulsifiers, enzymes, enzyme inhibitors, essential oils, fibers, film-forming agents, fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, dyes, friction-reducing agents, lubricants, moisturizing creams, ointments, opacifying agents, plasticizing agents, covering agents, polish, preservatives, gloss agents, green and synthetic polymers, powders, proteins, re-oiling agents, abrading agents, silicones, skin-soothing agents, skin-cleansing agents, skin care agents, skin-healing agents, skin-lightening agents, skin-protecting agents, skin-softening agents, hair promotion agents, cooling agents, skin-cooling agents, warming agents, skin-warming agents, stabilizers, surfactants, UV-absorbing agents, UV filters, primary sun protection factors, secondary sun protection factors, detergents, fabric conditioning agents, suspending agents, skin-tanning agents, actives modulating skin or hair pigmentation, matrix-metalloproteinase inhibitors, skin moisturizing agents, glycosaminoglycan stimulators, TRPV1 antagonists, desquamating agents, anti-cellulite agents or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxy fatty acids, liquefiers, dyestuffs, colour-protecting agents, pigments, anti-corrosives, fragrances or perfume oils, aromas, flavouring substances, odoriferous substances, polyols, electrolytes, organic solvents, and mixtures of two or more of the aforementioned substances, as further described below.

Of the above cosmetically or pharmaceutically active agents and/or adjuvants and/or additives or auxiliary agents against ageing of the skin, anti-microbial agents, antioxidants, chelating agents, emulsifiers, surfactants, preservatives, green and synthetic polymers, skin-cooling agents, rheology additives, oils, fragrances or perfume oils, and polyols are particularly preferred in the preparation of cosmetic and pharmaceutical composition.

Since dermatological conditions or diseases are often associated with dry skin, scratched skin, skin lesions or even inflammation, the cosmetic or pharmaceutical composition according to the present invention advantageously contains preferably anti-inflammatories, antibacterial or antimycotic substances, substances having a reddening-alleviating or itch-alleviating action, lenitive substances, moisturisers and/or cooling agents, osmolytes, keratolytic substances, nurturing substances, anti-inflammatory, antibacterial or antimycotic substances, substances having a reddening-alleviating or itch-alleviating action, lenitive substances, antidandruff substances, or other active compounds such as solvents, fragrances antioxidants, preservatives, (metal) chelating agents, penetration enhancers, or mixtures of two or more of afore specified agents, as further described below.

Anti-ageing actives: The cosmetic or pharmaceutical composition according to the present invention preferably contains one or more anti-ageing actives. In the context of the invention, anti-ageing or biogenic agents are, for example antioxidants, matrix-metalloproteinase inhibitors (MMPI), skin moisturizing agents, glycosaminglycan stimulators, anti-inflammatory agents, TRPV1 antagonists and plant extracts.

Antioxidants: A preferred cosmetic or pharmaceutical composition according to the present invention comprises one or more antioxidants. Suitable antioxidants encompass amino acids (preferably glycine, histidine, tyrosine, tryptophan) and derivatives thereof, imidazoles (preferably urocanic acid) and derivatives thereof, peptides, preferably D,L-carnosine, D-carnosine, L-carnosine and derivatives thereof (preferably anserine), carnitine, creatine, matrikine peptides (preferably lysyl-threonyl-threonyl-lysyl-serine) and palmitoylated pentapeptides, carotenoids, carotenes (preferably alpha-carotene, beta-carotene, lycopene) and derivatives thereof, lipoic acid and derivatives thereof (preferably dihydrolipoic acid), aurothioglucose, propyl thiouracil and other thiols (preferably thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl, glyceryl and oligoglyceryl esters thereof) and salts thereof, dilauryl thiodipropionate, distearyl thiodipropionate, thiodipropionic acid and derivatives thereof (preferably esters, ethers, peptides, lipids, nucleotides, nucleosides and salts) and sulfoximine compounds (preferably buthionine sulfoximines, homocysteine sulfoximine, buthionine sulfones, penta-, hexa-, heptathionine sulfoximine) in very small tolerated doses (e.g. pmol to µmol/kg), also (metal) chelators (preferably alpha-hydroxy fatty acids, palmitic acid, phytic acid, lactoferrin, alpha-hydroxy acids (preferably citric acid, lactic acid, malic acid), humic acid, bile acid, bile extracts, tannins, bilirubin, biliverdin, EDTA, EGTA and derivatives thereof), unsaturated fatty acids and derivatives thereof (preferably gamma-linolenic acid, linoleic acid, oleic acid), folic acid and derivatives thereof, ubiquinone and derivatives thereof, ubiquinol and derivatives thereof, vitamin C and derivatives (preferably ascorbyl palmitate, Mg ascorbyl phosphate, ascorbyl acetate, ascorbyl glucoside), tocopherols and derivatives (preferably vitamin E acetate), vitamin A and derivatives (vitamin A palmitate) and coniferyl benzoate of benzoic resin, rutinic acid and derivatives thereof, flavonoids and glycosylated precursors thereof, in particular quercetin and derivatives thereof, preferably alpha-glucosyl rutin, rosmarinic acid, carnosol, carnosolic acid, resveratrol, caffeic acid and derivatives thereof, sinapic acid and derivatives thereof, ferulic acid and derivatives thereof, curcuminoids, chlorogenic acid and derivatives thereof, retinoids, preferably retinyl palmitate, retinol or tretinoin, ursolic acid, levulinic acid, butyl hydroxytoluene, butyl hydroxyanisole, nordihydroguaiac acid, nordihydroguaiaretic acid, trihydroxybutyrophenone, uric acid and derivatives thereof, mannose and derivatives thereof, zinc and derivatives thereof (preferably ZnO, ZnSO₄), selenium and derivatives thereof (preferably selenium methionine), superoxide dismutase, stilbenes and derivatives thereof (preferably stilbene oxide, trans-stilbene oxide) and the derivatives (salts, esters, ethers, sugars, nucleotides, nucleosides, peptides and lipids) of these cited active ingredients which are suitable according to the invention or extracts or fractions of plants having an antioxidant effect, preferably green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John’s wort, grape seeds, wheat germ, Phyllanthus emblica, coenzymes, preferably coenzyme Q10, plastoquinone and menaquinone. Preferred antioxidants are selected from the group consisting of vitamin A and derivatives, vitamin C and derivatives, tocopherol and derivatives, preferably tocopheryl acetate, and ubiquinone. If vitamin E and/or derivatives thereof are used as the antioxidant(s), it is advantageous to choose their concentrations from the range from about 0.001 to about 10% by weight, based on the total weight of the composition. If vitamin A or vitamin A derivatives or carotenes or derivatives thereof are used as the antioxidant(s), it is advantageous to choose their concentrations from the range from about 0.001 to about 10% by weight based on the total weight of the composition.

Matrix-Metalloproteinase inhibitors (MMPI): A preferred cosmetic or pharmaceutical composition according to the present invention comprises one or more matrix-metalloproteinase inhibitors, especially those inhibiting matrix-metalloproteinases enzymatically cleaving collagen, selected from the group consisting of: ursolic acid, retinyl palmitate, propyl gallate, precocenes, 6-hydroxy-7-methoxy-2,2-dimethyl-1 (2H)-benzopyran, 3,4-dihydro-6-hydroxy-7-methoxy-2,2-dimethyl-1(2H)-benzopyran, benzamidine hydrochloride, the cysteine proteinase inhibitors N-ethylmalemide and epsilon-amino-n-caproic acid of the serinprotease inhibitors: phenylmethylsufonylfluoride, collhibin (company Pentapharm; INCI: hydrolysed rice protein), oenotherol (company Soliance; INCI: propylene glycol, aqua, Oenothera biennis root extract, ellagic acid and ellagitannins, for example from pomegranate), phosphoramidone hinokitiol, EDTA, galardin, EquiStat (company Collaborative Group; apple fruit extract, soya seed extract, ursolic acid, soya isoflavones and soya proteins), sage extracts, MDI (company Atrium; INCI: glycosaminoglycans), fermiskin (company Silab/Mawi; INCI: water and lentinus edodes extract), actimp 1.9.3 (company Expanscience/Rahn; INCI: hydrolysed lupine protein), lipobelle soyaglycone (company Mibelle; INCI: alcohol, polysorbate 80, lecithin and soy isoflavones), extracts from green and black tea and further plant extracts, proteins or glycoproteins from soya, hydrolysed proteins from rice, pea or lupine, plant extracts which inhibit MMPs, preferably extracts from shitake mushrooms, extracts from the leaves of the Rosaceae family, sub-family Rosoideae, quite particularly extracts of blackberry leaf, as e.g. SymMatrix (company Symrise, INCI: Maltodextrin, Rubus Fruticosus (Blackberry) Leaf Extract). Preferred actives of are selected from the group consisting of retinyl palmitate, ursolic acid, extracts from the leaves of the Rosaceae family, sub-family Rosoideae, genistein and daidzein.

Skin-moisturizing agents: A preferred cosmetic or pharmaceutical composition according to the present invention comprises one or more skin-moisturizing agents. Preferred skin moisturizing agents are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of: glycerol, 1,2-propylene glycol, 1,2-butylene glycol, 1,3-butylene glycol, 1,2-pentanediol, 1,2-hexanediol, 1,2-octanediol and 1,2-decanediol.

Glycosaminoglycan stimulators: A preferred cosmetic or pharmaceutical composition according to the present invention comprises one or more substances stimulating the synthesis of glycosaminoglycans which are selected from the group consisting of hyaluronic acid and derivatives or salts, Subliskin (Sederma, INCI: Sinorhizobium Meliloti Ferment Filtrate, Cetyl Hydroxyethylcellulose, Lecithin), Hyalufix (BASF, INCI: Water, Butylene Glycol, Alpinia galanga leaf extract, Xanthan Gum, Caprylic/Capric Triglyceride), Stimulhyal (Soliance, INCI: Calcium ketogluconate), Syn-Glycan (DSM, INCI: Tetradecyl Aminobutyroylvalylaminobutyric Urea Trifluoroacetate, Glycerin, Magnesium chloride), Kalpariane (Biotech Marine), DC Upregulex (Distinctive Cosmetic Ingredients, INCI: Water, Butylene Glycol, Phospholipids, Hydrolyzed Sericin), glucosamine, N-acetyl glucosamine, retinoids, preferably retinol and vitamin A, Arctium lappa fruit extract, Eriobotrya japonica extract, Genkwanin, N-Methyl-L-serine, (-)-alpha-bisabolol or synthetic alpha-bisabolol such as e.g. Dragosantol and Dragosantol 100 from Symrise, oat glucan, Echinacea purpurea extract and soy protein hydrolysate. Preferred actives are selected from the group consisting of hyaluronic acid and derivatives or salts, retinol and derivatives, (-)-alpha-bisabolol or synthetic alpha-bisabolol such as e.g. Dragosantol and Dragosantol 100 from Symrise, oat glucan, Echinacea purpurea extract, Sinorhizobium Meliloti Ferment Filtrate, Calcium ketogluconate, Alpinia galanga leaf extract and tetradecyl aminobutyroylvalylaminobutyric urea trifluoroacetate.

TRPV1 antagonists: A preferred cosmetic or pharmaceutical composition according to the present invention comprises one or more TRPV1 antagonsits. Suitable compounds which reduce the hypersensitivity of skin nerves based on their action as TRPV1 antagonists, encompass e.g. trans-4-tert-butyl cyclohexanol, or indirect modulators of TRPV1 by an activation of the µ-receptor, e.g. acetyl tetrapeptide-15, are preferred.

Plant extracts: A preferred cosmetic or pharmaceutical composition according to the present invention comprises one or more plant extracts. Plant extracts, special highly active plant extract fractions and also highly pure active substances isolated from plant extracts can also be used in the cosmetic or pharmaceutical composition according to the present invention. Extracts, fractions and active substances from camomile, aloe vera, Commiphora species, Rubia species, willows, willow-herb, ginger, marigold, arnica, Glycyrrhiza species, Echinacea species, Rubus species and pure substances such as inter alia bisabolol, apigenin, apigenin-7-glucoside, gingerols such as [6]-gingerol, paradols such as [6]-paradol, boswellic acid, phytosterols, glycyrrhizine, glabridin or licochalcone A are particularly preferred.

Anti-inflammatory agents: The cosmetic or pharmaceutical composition according to the present invention preferably also contains anti-inflammatory and/or redness and/or itch ameliorating ingredients, in particular steroidal substances of the corticosteroid type selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatories. Non-steroidal anti-inflammatories can also be used. More particularly:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John’s wort, honeysuckle, rosemary, Passiflora incarnata, witch hazel, ginger or Echinacea, or single active compounds thereof,
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives are preferred anti-itch ingredients in a composition according to the present invention.

Also useful are natural or naturally occurring anti-inflammatory mixtures of substances or mixtures of substances that alleviate reddening and/or itching, in particular extracts or fractions from camomile, Aloe vera, Commiphora species, Rubia species, willow, willow-herb, oats, calendula, arnica, St John’s wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occuring avenanthramides, preferably tranilast, avenanthramide A, avenanthramide B, avenanthramide C, non-natural or non-naturally occuring avenanthramides, preferably dihydroavenanthramide D, dihydroavenanthramide E, avenanthramide D, avenanthramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-)ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and β-glucans, in particular 1,3->1,4-β-glucan from oats.

When bisabolol is used in the context of the present invention it can be of natural or synthetic origin and is preferably “alpha-bisabolol”. Preferably, the bisabolol used is synthetically prepared or natural (-)-alpha-bisabolol and/or synthetic mixed-isomer alpha-bisabolol. If natural (-)-alpha-bisabolol is used, this can also be employed as a constituent of an essential oil or of a plant extract or of a fraction thereof, for example as a constituent of (fractions of) oil or extracts of camomile or of Vanillosmopsis (in particular Vanillosmopsis erythropappa or Vanillosmopsis arborea). Synthetic alpha-bisabolol is obtainable, for example, under the name “Dragosantol” from Symrise.

In case ginger extract is used in the context of the present invention, preferably extracts of the fresh or dried ginger root are used which are prepared by extraction with methanol, ethanol, iso-propanol, acetone, ethyl acetate, carbon dioxide (CO₂), hexane, methylene chloride, chloroform or other solvents or solvent mixtures of comparable polarity. The extracts are characterized by the presence of active skin irritation-reducing amounts of constituents such as e.g. gingerols, shogaols, gingerdiols, dehydrogingerdiones and/or paradols.

Physiological cooling agents: The cosmetic or pharmaceutical composition according to the present invention can be particularly advantageously combined with one or more physiological cooling agent(s). The use of cooling agents can alleviate itching. Preferred individual cooling agents for use within the framework of the present invention are listed below. The person skilled in the art can add many other cooling agents to this list; the cooling agents listed can also be used in combination with one another: which are preferably selected here from the following list: menthol and menthol derivatives (for example L-menthol, D-menthol, racemic menthol, isomenthol, neoisomenthol, neomenthol) menthylethers (for example (I-menthoxy)-1,2-propanediol, (I-menthoxy)-2-methyl-1,2-propanediol, I-menthyl-methylether), menthone glyceryl acetal, menthone glyceryl ketal or mixtures of both, menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy)acetate, menthylpyroglutamate), menthylcarbonates (for example menthylpropyleneglycolcarbonate, menthylethyleneglycolcarbonate, menthylglycerolcarbonate or mixtures thereof), the semi-esters of menthols with a dicarboxylic acid or derivatives thereof (for example mono-menthylsuccinate, mono-menthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or N^{α}-(menthanecarbonyl)glycinethylester [WS5], menthanecarboxylic acid-N-(4-cyanophenyl)amide or menthanecarboxylic acid-N-(4-cyanomethylphenyl)amide, menthanecarboxylic acid-N-(alkoxyalkyl)amides), menthone and menthone derivatives (for example L-menthone glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (I-(-)-isopulegol, I-(-)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2(1-pyrrolidinyl)-2-cyclopentene-1-one) or tetrahydropyrimidine-2-one (for example iciline or related compounds, as described in WO 2004/026840), further carboxamides (for example N-(2-(pyridin-2-yl)ethyl)-3-p-menthanecarboxamide or related compounds), (1R,2S,5R)-N-(4-Methoxyphenyl)-5-methyl-2-(1-isopropyl)cyclohexane-carboxamide [WS12], oxamates and [(1R,2S,5R)-2-isopropyl-5-methyl-cyclohexyl] 2-(ethylamino)-2-oxo-acetate (X Cool). Cooling agents which are preferred due to their particular synergistic effect are I-menthol, d-menthol, racemic menthol, menthone glycerol acetal (trade name: Frescolat® MGA), menthyl lactate (preferably I-menthyl lactate, in particular I-menthyl I-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are I-menthol, racemic menthol, menthone glycerol acetal (trade name: Frescolat® MGA), menthyl lactate (preferably I-menthyl lactate, in particular I-menthyl I-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are I-menthol, menthone glycerol acetal (trade name: Frescolat® MGA) and menthyl lactate (preferably I-menthyl lactate, in particular I-menthyl I-lactate (trade name: Frescolat® ML).

Moisturising and/or moisture-retaining substances: Itching occurs with particular intensity when the skin is dry. The use of skin-moisturising and/or moisture-retaining substances can significantly alleviate itching. The cosmetic or pharmaceutical composition according to the present invention can therefore advantageously also contain one or more of the following moisturising and/or moisture-retaining substances: sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Lenitive substances: The cosmetic or pharmaceutical composition according to the present invention can also contain advantageously one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica monatana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

Antibacterial or antimycotic active substances: Antibacterial or antimycotic active substances can also particularly advantageously be used in the cosmetic or pharmaceutical composition according to the present invention, wherein any antibacterial or antimycotic active substances can be used which are suitable or customary in cosmetic or pharmaceutical, in particular dermatological applications. In addition to the large group of conventional antibiotics, other products which are advantageous here include those relevant to cosmetics such as in particular triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate or combinations of said substances, which are used inter alia against underarm odour, foot odour or dandruff.

Anti-microbial agents: The cosmetic or pharmaceutical composition according to the present invention preferably contains one or more anti-microbioal agents. Suitable anti-microbial agents are, in principle, all substances effective against Gram-positive bacteria, such as, for example, 4-hydroxybenzoic acid and its salts and esters, N-(4-chlorophenyl)-N′-(3,4- dichlorophenyl)urea, 2,4,4′-trichloro-2′-hydroxydiphenyl ether (triclosan), 4-chloro-3,5-dimethyl-phenol, 2,2′-methylenebis(6-bromo-4-chlorophenol), 3-methyl-4-(1-methylethyl)phenol, 2-benzyl-4-chloro-phenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

Desquamating agents: The cosmetic or pharmaceutical composition according to the present invention preferably contains one or more desquamating agents. The expression “desquamating agent” is understood to mean any compound capable of acting:
either directly on desquamation by promoting exfoliation, such as β-hydroxy acids, in particular salicylic acid and its derivatives (including 5-n-octanoylsalicylic acid); α-hydroxy acids, such as glycolic, citric, lactic, tartaric, malic or mandelic acids; urea; gentisic acid; oligofucoses; cinnamic acid; extract of Sophora japonica; resveratrol and some derivatives of jasmonic acid;
or on the enzymes involved in the desquamation or the degradation of the corneodesmosomes, glycosidases, stratum corneum chymotryptic enzyme (SCCE) or other proteases (trypsin, chymotrypsin-like). There may be mentioned agents chelating inorganic salts: EDTA; N-acyl-N,N′,N′-ethylenediaminetriacetic acid; aminosulphonic compounds and in particular (N-2-hydroxyethylpiperazine-N-2-ethane)sulphonic acid (HEPES); derivatives of 2-oxothiazolidine-4-carboxylic acid (procysteine); derivatives of alpha-amino acids of the glycine type (as described in EP-0 852 949, and sodium methylglycine diacetate marketed by BASF under the trade name TRILON M); honey; sugar derivatives such as O-octanoyl-6-D-maltose and N-acetylglucosamine; chestnut extracts such as those marketed by the company SILAB under the name Recoverine®, prickly pear extracts such as those marketed under the name Exfolactive® by the company SILAB, or Phytosphingosine SLC® (phytosphingosine grafted with a salicylic acid) marketed by the company Degussa.

Desquamating agents suitable for the invention may be chosen in particular from the group comprising sulphonic acids, calcium chelators, α-hydroxy acids such as glycolic, citric, lactic, tartaric, malic or mandelic acids; ascorbic acid and its derivatives such as ascorbyl glucoside and magnesium ascorbyl phosphate; nicotinamide; urea; (N-2-hydroxyethylpiperazine-N-2-ethane)sulphonic acid (HEPES), β-hydroxy acids such as salicylic acid and its derivatives, retinoids such as retinol and its esters, retinal, retinoic acid and its derivatives, chestnut or prickly pear extracts, in particular marketed by SILAB; reducing compounds such as cysteine or cysteine precursors.

Desquamating agents which can be used are also nicotinic acid and its esters and nicotinamide, also called vitamin B3 or vitamin PP, and ascorbic acid and its precursors.

Anti-dandruff substances: In addition, the cosmetic or pharmaceutical composition according to the present invention can also advantageously be used in combination with one or more anti-dandruff substances, including triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate, Propanediol Monocaprylate or combinations of said substances, which are used inter alia against dandruff.

Further suitable anti-dandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

(Metal) chelating agents: A combination with one or more (metal) chelating agents can also be advantageous used in the cosmetic or pharmaceutical composition according to the present invention, wherein any metal chelating agents can be used which are suitable or customary in cosmetic or pharmaceutical applications. Preferred (metal) chelating agents include α-hydroxy fatty acids, phytic acid, lactoferrin, α-hydroxy acids, such as inter alia gluconic acid, glyceric acid, glycolic acid, isocitric acid, citric acid, lactic acid, malic acid, mandelic acid, tartaric acid, as well as humic acids, bile acids, bile extracts, bilirubin, biliverdin or EDTA, EGTA and their derivatives. The use of one or more chelating agent(s) improves the stability of the composition according to the present invention.

Emulsifiers: In addition, the cosmetic or pharmaceutical composition according to the present invention can also advantageously contain one or more emulsifiers, including for example:
products of the addition of 2 to 30 mol ethylene oxide and/or 0 to 5 mol propylene oxide onto linear C₈-₂₂ fatty alcohols, onto C₁₂-₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol monoand diesters and sorbitan monoand diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, diand/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆-₂₂ fatty acids, methyl glucose and polyols, preferably glycerol or polyglycerol;
polyalkylene glycols; and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate, and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic emulsifiers: Typical anionic emulsifiers are aliphatic C12 to C 22 fatty acids, such as palmitic acid, stearic acid or behenic acid for example, and C12 to C22 dicarboxylic acids, such as azelaic acid or sebacic acid for example.

Amphoteric emulsifiers: Other suitable emulsifiers are amphoteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine.

Surfactants: For the preparation of solid surfactant compositions, the cosmetic or pharmaceutical composition according to the present invention preferably includes one or more surfactant(s) which is/are commonly used therefor. Suitable surfactants include Sodium Laureth sulfate, Cocamidopropyl Betaine, Sodium Stearate, Sodium Lauryl Sulfate, Glycol distearate, Coco-Glucoside, Sodium Coco-Sulfate, Sodium Cocoate, Coco-Betaine, Cocamide MEA, Lauryl Glucoside, PEG-7 Glyceryl Cocoate, Decyl Glucoside, Potassium Lauryl Sulfate, Sodium Palmate, Sodium Palm Kernelate, Sodium Cocoyl Glutamate, Disodium Laureth Sulfosuccinate, Sodium lauroyl sarcosinate, Sodium Lauroyl Sarcosinate, Sodium Cocoyl Isethionate, Sodium cocoamphoacetate, Sodium Methyl Cocoyl Taurate, Caprylyl/Capryl Glucoside, Disodium Lauryl Sulfosuccinate, Sodium Lauryl Sulfosuccinate, PPG-5-ceteth-20, Polyglyceryl-4 Laurate, Sodium Lauryl Sulfoacetate, Sodium Lauryl Sulfoacetate, Sodium Myristoyl Glutamate, Alpha Olefin Sulfonate, Diethylhexyl Sodium Sulfosuccinate, Ammonium Cocoyl Isethionate, Sodium Oleoyl Sarcosinate, Ammonium Cocoyl Sarcosinate, or mixtures of one or more of the aforesaid surfactants.

Preferred surfactants for the preparation of solid surfactant compositions according to the present invention are selected from the group consisting of Sodium Stearate, Sodium Lauryl Sulfate, Coco-Glucoside, Sodium Coco-Sulfate, Sodium Cocoate, Coco-Betaine, Lauryl Glucoside, Decyl Glucoside, Potassium Lauryl Sulfate, Sodium Palmate, Sodium Palm Kernelate, Sodium Cocoyl Glutamate, Disodium Laureth sulfosuccinate, Sodium lauroyl sarcosinate, Sodium Lauroyl Sarcosinate, Sodium Cocoyl Isethionate, Sodium Cocoamphoacetate, Sodium Methyl Cocoy Taurate, Caprylyl/Capryl glucoside, Disodium Lauryl Sulfosuccinate, and mixtures of two or more of the aforesaid surfactants.

Preservatives: For preservative purposes, the cosmetic or pharmaceutical composition according to the present invention preferably includes one or more preservatives which are suitable or customary in cosmetic or pharmaceutical composition. Suitable and advantageously preservatives are, for example, phenoxyethanol, formaldehyde solution, parabens, pentanediol or sorbic acid.

Green and synthetic polymers: The cosmetic or pharmaceutical composition according to the present invention preferably includes one or more green or synthetic polymers. Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol®A-15, Mirapol® AD-1, Mirapol® AZ-1 of Miranol and the various polyquaternium types (for example 6, 7, 32 or 37) which can be found in the market under the tradenames Rheocare® CC or Ultragel® 300. Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. In a preferred variant, the cosmetic or pharmaceutical composition according to the present invention preferably includes an uncrosslinked or polyol-crosslinked polyacrylic acid as polymer component.

Thickening agents and/or rheology additives: The cosmetic or pharmaceutical composition according to the present invention preferably includes one or more thickening agents and/or rheology additives. Suitable thickeners are polymeric thickeners, such as Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates and electrolytes, such as sodium chloride and ammonium chloride.

Perfume oils and/or fragrances: The cosmetic or pharmaceutical composition according to the present invention preferably includes one or more perfume oils and/or fragrances. Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, beta-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Anti-cellulite agent: The cosmetic or pharmaceutical composition according to the present invention preferably includes one or more anti-cellulite agents. Anti-cellulite agents and lipolytic agents are preferably selected from the group consisting of beta-adrenergic receptor agonists such as synephrine and its derivatives, and cyclohexyl carbamates. Agents enhancing or boosting the activity of anti-cellulite agents, in particular agents which stimulate and/or depolarise C nerve fibres, are preferably selected from the group consisting of capsaicin and derivatives thereof, vanillyl-nonylamid and derivatives thereof, L-carnitine, coenzym A, isoflavonoides, soy extracts, ananas extract and conjugated linoleic acid.

Fat enhancing agents: The cosmetic or pharmaceutical composition according to the present invention preferably includes one or more fat enhancing agents and/or adipogenic agents as well as agents enhancing or boosting the activity of fat enhancing agents. A fat enhancing agent is for example hydroxymethoxyphenyl propylmethylmethoxybenzofuran (trade name: Sym3D®).

Oil bodies: In addition to the liquid lipophilic component, the cosmetic or pharmaceutical composition according to the present invention preferably includes one or more oil bodies. Suitable oil bodies, which form constituents of the O/W emulsions, are, for example, Guerbet alcohols based on fatty alcohols having 6 to 18, preferably 8 to 10, carbon atoms, esters of linear C5 - C22 fatty acids with linear or branched C5 - C22 fatty alcohols or esters of branched C1 - C13 carboxylic acids with linear or branched C6 - C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6 - C22 fatty acids with branched alcohols, in particular 2-ethylhexanol, esters of C18 - C38 alkylhydroxy carboxylic acids with linear or branched C6 - C22 fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C6 - C10 fatty acids, liquid mono-/di-/triglyceride mixtures based on C6 - C18 fatty acids, esters of C6 -C22 fatty alcohols and/or Guerbet alcohols with aromatic carboxylic acids, in particular benzoic acid, esters of C2 - C12 dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched CC - C22 fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes.

Superfatting agents and/or consistency factors: The cosmetic or pharmaceutical composition according to the present invention preferably includes one or more superfatting agents and/or consistency factors. Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers. The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Pearlizing waxes: The cosmetic or pharmaceutical composition according to the present invention preferably includes one or more pearlizing waxes. Suitable pearlising waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups, and mixtures thereof.

Silicones: In order to impart a silky, spreadable, and luxurious texture and to make skin look and feel smoother, and additionally to improve processability (antifoaming) the cosmetic or pharmaceutical composition according to the present invention preferably includes one or more silicones or silicone deratives. Suitable silicones can be chosen from the group consisting of Acefylline Methylsilanol Mannuronate, Acetylmethionyl Methylsilanol Elastinate Acrylates/Behenyl, Acrylate/Dimethicone Methacrylate Copolymer, Acrylates/Behenyl Methacrylate/Dimethicone Methacrylate Copolymer, Acrylates/Bis-Hydroxypropyl Dimethicone Crosspolymer, Acrylates/Dimethicone Copolymer, Acrylates/Dimethicone Methacrylate/Ethylhexyl Acrylate Copolymer, Acrylates/Dimethiconol Acrylate Copolymer, Acrylates/Ethylhexyl Acrylate/Dimethicone Methacrylate Copolymer, Acrylates/Octylacrylamide/Diphenyl Amodimethicone Copolymer, Acrylates/Polytrimethylsiloxymethacrylate Copolymer, Acrylates/Propyl Trimethicone Methacrylate Copolymer, Acrylates/Stearyl Acrylate/Dimethicone Methacrylate Copolymer, Acrylates/Tridecyl Acrylate/Triethoxysilylpropyl Methacrylate/Dimethicone Methacrylate Copolymer, Acrylates/Trifluoropropylmethacrylate/Polytrimethyl Siloxymethacrylate Copolymer, Amino Bispropyl Dimethicone, Aminoethylaminopropyl Dimethicone, Aminopropyl Dimethicone, Aminopropyl Phenyl Trimethicone, Aminopropyl Triethoxysilane, Ammonium Dimethicone PEG-7 Sulfate, Amodimethicone, Amodimethicone Hydroxystearate, Amodimethicone/Silsesquioxane Copolymer, Ascorbyl Carboxydecyl Trisiloxane, Ascorbyl Methylsilanol Pectinate, Behenoxy Dimethicone, Behentrimonium Dimethicone PEG-8 Phthalate, Behenyl Dimethicone, Bisamino PEG/PPG-41/3 Aminoethyl PG-Propyl Dimethicone, Bis-Aminopropyl/Ethoxy Aminopropyl Dimethicone, Bis(Butylbenzoate) Diaminotriazine Aminopropyltrisiloxane, Bis-Butyldimethicone Polyglyceryl-3, Bis-Butyloxyamodimethicone/PEG-60 Copolymer, Bis(C13-15 Alkoxy) Hydroxybutamidoamodimethicone, Bis(C13-15 Alkoxy) PG- Amodimethicone, Bis-(C1-8 Alkyl Lauroyl Lysine Decylcarboxamide) Dimethicone, Bis-Cetyl Cetyl Dimethicone, Bis-Cetyl/PEG-8 Cetyl PEG-8 Dimethicone, Bis-Diphenylethyl Disiloxane, Bis-Ethyl Ethyl Methicone, Bis-Gluconamidoethylaminopropyl Dimethicone, Bis-Hydrogen Dimethicone, Bis- Hydroxyethoxypropyl Dimethicone Bis-Hydroxylauryl, Dimethicone/IPDI Copolymer, Bis- Hydroxy/Methoxy Amodimethicone, Bis-Hydroxypropyl Dimethicone Behenate, Bis-Hydroxypropyl Dimethicone/SMDI Copolymer, Bis-Isobutyl PEG-14/Amodimethicone Copolymer, Bis-Isobutyl PEG-15/Amodimethicone Copolymer, Bis-Isobutyl PEG/PPG-20/35/Amodimethicone Copolymer, Bis- Isobutyl PEG/PPG-10/7/Dimethicone Copolymer, Bis-Isobutyl PEG-24/PPG-7/Dimethicone Copolymer, Bis-PEG-1 Dimethicone, Bis-PEG-4 Dimethicone, Bis-PEG-8 Dimethicone, Bis-PEG-12 Dimethicone,Bis-PEG-20 Dimethicone, Bis-PEG-12 Dimethicone Beeswax, Bis-PEG-12 Dimethicone Candelillate, Bis-PEG-15 Dimethicone/IPDI Copolymer, Bis-PEG-15 Methyl Ether Dimethicone, Bis- PEG-18 Methyl Ether Dimethyl Silane, Bis-PEG/PPG-14/14 Dimethicone, Bis-PEG/PPG-15/5 Dimethicone, Bis-PEG/PPG-18/6 Dimethicone, Bis-PEG/PPG-20/20 Dimethicone, Bis-PEG/PPG- 16/16 PEG/PPG-16/16 Dimethicone, Bis-PEG/PPG-20/5 PEG/PPG-20/5 Dimethicone, Bisphenylhexamethicone, Bis-Phenylpropyl Dimethicone, Bispolyethylene Dimethicone, Bis- (Polyglyceryl-3 Oxyphenylpropyl) Dimethicone, Bis-(Polyglyceryl-7 Oxyphenylpropyl) Dimethicone, Bis-PPG-15 Dimethicone/IPDI Copolymer, Bis(PPG-7 Undeceneth-21) Dimethicone, Bis-Stearyl Dimethicone, Bis-Trimethoxysilylethyl Tetramethyldisiloxyethyl Dimethicone, Bis-Vinyldimethicone, Bis-Vinyl Dimethicone/Dimethicone Copolymer, Borage Seed Oil PEG-7 Dimethicone Esters, Butyl Acrylate/C6-14 Perfluoroalkylethyl Acrylate/Mercaptopropyl Dimethicone Copolymer, Butyl Acrylate/Hydroxypropyl Dimethicone Acrylate Copolymer, Butyl Dimethicone Acrylate/Cyclohexylmethacrylate/Ethylhexyl Acrylate Copolymer, Butyldimethicone Methacrylate/Methyl Methacrylate Crosspolymer, t-Butyl Dimethyl Silyl Grape Seed Extract, Butyl Polydimethylsiloxyl Ethylene/Propylene/Vinylnorbornene Copolymer, C6-8 Alkyl C3-6 Alkyl Glucoside Dimethicone, C20-24 Alkyl Dimethicone,C24-28 Alkyl Dimethicone, C26-28 Alkyl Dimethicone, C30-45 Alkyl Dimethicone, C30-60 Alkyl Dimethicone, C32 Alkyl Dimethicone, C30-45 Alkyl Dimethicone/Polycyclohexene Oxide Crosspolymer, C26-28 Alkyldimethylsilyl Polypropylsilsesquioxane, C30-45 Alkyldimethylsilyl Polypropylsilsesquioxane, C20-24 Alkyl Methicone, C24-28 Alkyl Methicone, C26-28 Alkyl Methicone, C30-45 Alkyl Methicone, C20-28 Alkyl Perfluorodecylethoxy Dimethicone, C26-54 Alkyl Tetradecyl Dimethicone, Capryl Dimethicone, Caprylyl Dimethicone Ethoxy Glucoside, Caprylyl Methicone, Caprylyl Trimethicone, Carboxydecyl Trisiloxane, Castor Oil Bis-Hydroxypropyl Dimethicone Esters Cerotyl Dimethicone, Cetearyl Dimethicone Crosspolymer, Cetearyl Dimethicone/Vinyl Dimethicone Crosspolymer, Cetearyl Methicone, Cetrimonium Carboxydecyl PEG-8 Dimethicone, Cetrimonium Dimethicone PEG-7 Phthalate, Cetyl Behenyl Dimethicone, Cetyl Dimethicone, Cetyl Dimethicone/Bis-Vinyldimethicone Crosspolymer, Cetyl Hexacosyl Dimethicone, Cetyloxy Dimethicone, Cetyl PEG-8 Dimethicone, Cetyl PEG/PPG-15/15 Butyl Ether Dimethicone, Cetyl PEG/PPG-7/3 Dimethicone, Cetyl PEG/PPG-10/1 Dimethicone, Cetyl Triethylmonium Dimethicone PEG-8 Phthalate, Cetyl Triethylmonium Dimethicone PEG-8 Succinate, Copper Acetyl Tyrosinate Methylsilanol, Copper PCA Methylsilanol, C4-14 Perfluoroalkylethoxy Dimethicone, Cycloethoxymethicone, Cycloheptasiloxane, Cyclohexasiloxane, Cyclomethicone, Cyclopentasiloxane, Cyclophenylmethicone, Cyclotetrasiloxane,mCyclovinylmethicone, Cystine Bis-PG-Propyl Silanetriol, DEA PG-Propyl PEG/PPG-18/21 Dimethicone, Diisostearoyl Trimethylolpropane Siloxy Silicate, Dilauroyl Trimethylolpropane Siloxy Silicate, Dilinoleamidopropyl Dimethylamine Dimethicone PEG-7 Phosphate, Dimethicone, Dimethicone Crosspolymer, Dimethicone Crosspolymer-3, Dimethicone/Divinyldimethicone/Silsesquioxane Crosspolymer, Dimethicone Ethoxy Glucoside, Dimethicone Hydroxypropyl Trimonium Chloride, Dimethicone/Mercaptopropyl Methicone Copolymer, Dimethicone PEG-15 Acetate Dimethicone PEG-8 Adipate, Dimethicone PEG-7 Avocadoate, Dimethicone PEG-8 Avocadoate, Dimethicone PEG-8 Beeswax, Dimethicone PEG-8 Benzoate, Dimethicone PEG-8 Borageate, Dimethicone PEG-7 Cocoate, Dimethicone/PEG-10 Crosspolymer, Dimethicone/PEG-10/15 Crosspolymer, Dimethicone/PEG-15 Crosspolymer, Dimethicone PEG-7 Isostearate, Dimethicone PEG-8 Isostearate, Dimethicone PEG-7 Lactate, Dimethicone PEG-8 Lanolate, Dimethicone PEG-8 Laurate, Dimethicone PEG-8 Meadowfoamate, Dimethicone PEG-7 Octyldodecyl Citrate, Dimethicone PEG-7 Olivate, Dimethicone PEG-8 Olivate, Dimethicone PEG-7 Phosphate, Dimethicone PEG-8 Phosphate, Dimethicone PEG-10 Phosphate, Dimethicone PEG-7 Phthalate, Dimethicone PEG-8 Phthalate, Dimethicone PEG-8 Polyacrylate, Dimethicone PEG/PPG- 20/23 Benzoate, Dimethicone PEG/PPG-7/4 Phosphate, Dimethicone PEG/PPG-12/4 Phosphate, Dimethicone PEG-7 Succinate, Dimethicone PEG-8 Succinate, Dimethicone PEG-7 Sulfate, Dimethicone PEG-7 Undecylenate, Dimethicone PG-Diethylmonium Chloride, Dimethicone/Phenyl Vinyl Dimethicone Crosspolymer, Dimethicone/Polyglycerin-3 Crosspolymer, Dimethicone/PPG-20 Crosspolymer, Dimethicone Propylethylenediamine Behenate, Dimethicone Propyl PG-Betaine, Dimethicone/Silsesquioxane Copolymer, Dimethicone Silylate, Dimethicone/Vinyldimethicone Crosspolymer, Dimethicone/Vinyltrimethylsiloxysilicate Crosspolymer, Dimethiconol, Dimethiconol Arginine, Dimethiconol Beeswax, Dimethiconol Behenate, Dimethiconol Borageate, Dimethiconol Candelillate, Dimethiconol Carnaubate, Dimethiconol Cysteine, Dimethiconol Dhupa Butterate, Dimethiconol Fluoroalcohol Dilinoleic Acid, Dimethiconol Hydroxystearate, Dimethiconol lllipe Butterate, Dimethiconol/IPDI Copolymer, Dimethiconol Isostearate, Dimethiconol Kokum Butterate, Dimethiconol Lactate, Dimethiconol Meadowfoamate, Dimethiconol Methionine, Dimethiconol/Methylsilanol/Silicate Crosspolymer, Dimethiconol Mohwa Butterate, Dimethiconol Panthenol, Dimethiconol Sal Butterate, Dimethiconol/Silica Crosspolymer, Dimethiconol/Silsesquioxane Copolymer, Dimethiconol Stearate, Dimethiconol/Stearyl, Methicone/Phenyl Trimethicone Copolymer, Dimethoxysilyl Ethylenediaminopropyl Dimethicone, Dimethylaminopropylamido PCA Dimethicone, Dimethyl Oxobenzo Dioxasilane, Dimethylsilanol Hyaluronate, Dioleyl Tocopheryl Methylsilanol, Diphenyl Amodimethicone, Diphenyl Dimethicone, Diphenyl Dimethicone Crosspolymer Diphenyl Dimethicone?/inyl Diphenyl Dimethicone/Silsesquioxane Crosspolymer, Diphenylethyl Benzyloxy Dilsiloxane, Diphenylisopropyl Dimethicone, Diphenylsiloxy Phenyl/Propyl Trimethicone, Diphenylsiloxy Phenyl Trimethicone Disiloxane, Disodium Amodimethicone Disuccinamide, Disodium PEG-12 Dimethicone Sulfosuccinate, Disodium PEG-8 Lauryl Dimethicone Sulfosuccinate, Divinyldimethicone/Dimethicone Copolymer, Divinyldimethicone/Dimethicone Crosspolymer, Drometrizole Trisiloxane, Ethylhexyl AcrylateNP/Dimethicone Methacrylate Copolymer, Ethyl Methicone, Ethyl Trisiloxane, Fluoro C2-8 Alkyldimethicone, Gluconamidopropyl Aminopropyl Dimethicone, 4-(2-Beta-Glucopyranosiloxy) Propoxy-2-Hydroxybenzophenone, Glyceryl Undecyl Dimethicone, Glycidoxy Dimethicone, Hexadecyl Methicone, Hexyl Dimethicone, Hexyl Methicone, Hexyltrimethoxysilane, Hydrogen Dimethicone, Hydrogen Dimethicone/Octyl Silsesquioxane Copolymer, Hydrolyzed Collagen PG-Propyl Dimethiconol, Hydrolyzed Collagen PG-Propyl Methylsilanediol, Hydrolyzed Collagen PG-Propyl Silanetriol, Hydrolyzed Keratin PG-Propyl Methylsilanediol, Hydrolyzed Sesame Protein PG-Propyl Methylsilanediol, Hydrolyzed Silk PG-Propyl Methylsilanediol, Hydrolyzed Silk PG-Propyl Methylsilanediol Crosspolymer, Hydrolyzed Soy Protein/Dimethicone PEG-7 Acetate, Hydrolyzed Soy Protein PG-Propyl Methylsilanediol, Hydrolyzed Vegetable Protein PG-Propyl Silanetriol, Hydrolyzed Wheat Protein/Cystine Bis-PG-Propyl Silanetriol Copolymer, Hydrolyzed Wheat Protein/Dimethicone PEG-7 Acetate, Hydrolyzed Wheat Protein/Dimethicone PEG-7 Phosphate Copolymer, Hydrolyzed Wheat Protein PG-Propyl Methylsilanediol, Hydrolyzed Wheat Protein PG-Propyl Silanetriol, Hydroxyethyl Acetomonium PG-Dimethicone, Hydroxypropyldimethicone, Hydroxypropyl Dimethicone Behenate, Hydroxypropyl Dimethicone Isostearate, Hydroxypropyl Dimethicone Stearate, Isobutylmethacrylate/Bis-Hydroxypropyl Dimethicone Acrylate Copolymer, Isobutylmethacrylate/Trifluoroethylmethacrylate/Bis-Hydroxypropyl Dimethicone Acrylate Copolymer, Isopentyl Trimethoxycinnamate Trisiloxane, Isopolyglyceryl-3 Dimethicone, Isopolyglyceryl-3 Dimethiconol, Isopropyl Titanium Triisostearate/Triethoxysilylethyl, Polydimethylsiloxyethyl Dimethicone Crosspolymer, Isostearyl Carboxydecyl PEG-8 Dimethicone, Lactoyl Methylsilanol Elastinate, Lauryl Dimethicone, Lauryl Dimethicone PEG-15 Crosspolymer, Lauryl Dimethicone PEG- 10 Phosphate, Lauryl Dimethicone/Polyglycerin-3 Crosspolymer, Lauryl Methicone, Lauryl PEG-8 Dimethicone, Lauryl PEG-10 Methyl Ether Dimethicone, Lauryl PEG-9 Polydimethylsiloxyethyl Dimethicone, Lauryl PEG/PPG-18/18 Methicone, Lauryl Phenylisopropyl Methicone, Lauryl Phenylpropyl Methicone, Lauryl Polydimethylsiloxyethyl Dimethicone/Bis-Vinyldimethicone Crosspolymer, Lauryl Polyglyceryl-3 Polydimethylsiloxyethyl Dimethicone, Lauryl Trimethicone, Linoleamidopropyl PG-Dimonium Chloride Phosphate Dimethicone, Methacryloyl Propyltrimethoxysilane, Methicone, Methoxy Amodimethicone/Silsesquioxane Copolymer, Methoxycinnamidopropyl Polysilsesquioxane, Methoxycinnamoylpropyl Silsesquioxane Silicate, Methoxy PEG-13 Ethyl Polysilsesquioxane, Methoxy PEG/PPG-7/3 Aminopropyl Dimethicone, Methoxy PEG/PPG-25/4 Dimethicone, Methoxy PEG-10 Propyltrimethoxysilane, Methyleugenyl PEG-8 Dimethicone, Methylpolysiloxane Emulsion, Methylsilanol Acetylmethionate, Methylsilanol Acetyltyrosine, Methylsilanol Ascorbate, Methylsilanol Carboxymethyl Theophylline, Methylsilanol Carboxymethyl Theophylline Alginate, Methylsilanol Elastinate, Methylsilanol Glycyrrhizinate, Methylsilanol Hydroxyproline, Methylsilanol Hydroxyproline Aspartate, Methylsilanol Mannuronate, Methylsilanol PCA, Methylsilanol PEG-7 Glyceryl Cocoate, Methylsilanol/Silicate Crosspolymer, Methylsilanol Spirulinate, Methylsilanol Tri-PEG-8 Glyceryl Cocoate, Methyl Trimethicone, Methyltrimethoxysilane, Myristylamidopropyl Dimethylamine Dimethicone PEG-7 Phosphate, Myristyl Methicone, Myristyl Trisiloxane, Nylon-611/Dimethicone Copolymer, PCA Dimethicone, PEG-7 Amodimethicone, PEG-8 Amodimethicone, PEG-8 Cetyl Dimethicone, PEG-3 Dimethicone, PEG-6 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG-10 Dimethicone Crosspolymer, PEG-12 Dimethicone Crosspolymer, PEG-8 Dimethicone Dimer Dilinoleate, PEG-8 Dimethicone/Dimer Dilinoleic Acid Copolymer, PEG-10 Dimethicone/Vinyl Dimethicone Crosspolymer, PEG-8 Distearmonium Chloride PG-Dimethicone, PEG-10/Lauryl Dimethicone Crosspolymer, PEG- 15/Lauryl Dimethicone Crosspolymer, PEG-15/Lauryl Polydimethylsiloxyethyl Dimethicone Crosspolymer, PEG-8 Methicone, PEG-6 Methicone Acetate, PEG-6 Methyl Ether Dimethicone, PEG- 7 Methyl Ether Dimethicone, PEG-8 Methyl Ether Dimethicone, PEG-9 Methyl Ether Dimethicone, PEG-10 Methyl Ether Dimethicone, PEG-11 Methyl Ether Dimethicone, PEG-32 Methyl Ether Dimethicone, PEG-8 Methyl Ether Triethoxysilane, PEG-10 Nonafluorohexyl Dimethicone Copolymer, PEG-4 PEG-12 Dimethicone, PEG-8 PG-Coco-Glucoside Dimethicone, PEG-9 Polydimethylsiloxyethyl Dimethicone, PEG/PPG-20/22 Butyl Ether Dimethicone, PEG/PPG-22/22 Butyl Ether Dimethicone, PEG/PPG-23/23 Butyl Ether Dimethicone, PEG/PPG-24/18 Butyl Ether Dimethicone, PEG/PPG-27/9 Butyl Ether Dimethicone, PEG/PPG-3/10 Dimethicone, PEG/PPG-4/12 Dimethicone, PEG/PPG-6/4 Dimethicone, PEG/PPG-6/11 Dimethicone, PEG/PPG-8/14 Dimethicone, PEG/PPG-8/26 Dimethicone, PEG/PPG-10/2 Dimethicone, PEG/PPG-12/16 Dimethicone, PEG/PPG- 12/18 Dimethicone, PEG/PPG-14/4 Dimethicone, PEG/PPG-15/5 Dimethicone, PEG/PPG-15/15 Dimethicone, PEG/PPG-16/2 Dimethicone, PEG/PPG-16/8 Dimethicone, PEG/PPG-17/18 Dimethicone, PEG/PPG-18/6 Dimethicone, PEG/PPG-18/12 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, PEG/PPG-30/10 Dimethicone, PEG/PPG-25/25 Dimethicone/Acrylates Copolymer, PEG/PPG-20/22 Methyl Ether Dimethicone, PEG/PPG-24/24 Methyl Ether Glycidoxy Dimethicone, PEG/PPG-10/3 Oleyl Ether Dimethicone, PEG/PPG-5/3 Trisiloxane, PEG-4 Trifluoropropyl Dimethicone Copolymer, PEG-8 Trifluoropropyl Dimethicone Copolymer, PEG-10 Trifluoropropyl Dimethicone Copolymer, PEG-8 Trisiloxane, Perfluorocaprylyl riethoxysilylethyl Methicone, Perfluorononyl Dimethicone, Perfluorononyl Dimethicone/Methicone/Amodimethicone Crosspolymer, Perfluorononylethyl Carboxydecyl Behenyl Dimethicone, Perfluorononylethyl Carboxydecyl Hexacosyl Dimethicone, Perfluorononylethyl Carboxydecyl Lauryl/Behenyl Dimethicone, Perfluorononylethyl Carboxydecyl Lauryl Dimethicone, Perfluorononylethyl Carboxydecyl PEG-8 Dimethicone, Perfluorononylethyl Carboxydecyl PEG-10 Dimethicone, Perfluorononylethyl Dimethicone/Methicone Copolymer, Perfluorononylethyl PEG-8 Dimethicone, Perfluorononylethyl Stearyl Dimethicone, Perfluorooctylethyl/Diphenyl Dimethicone Copolymer, Perfluorooctylethyl Triethoxysilane, Perfluorooctylethyl Trimethoxysilane, Perfluorooctylethyl Trisiloxane, Perfluorooctyl Triethoxysilane, PG-Amodimethicone, Phenethyl Dimethicone, Phenethyl Disiloxane, Phenyl Dimethicone, Phenylisopropyl Dimethicone, Phenyl Methicone, Phenyl Methiconol, Phenylpropyldimethylsiloxysilicate, Phenylpropyl Ethyl Methicone, Phenyl Propyl Trimethicone, Phenyl Propyl Trimethicone/Diphenylmethicone, Phenyl Trimethicone, Platinum Divinyldisiloxane, Polyacrylate-6, Polydiethylsiloxane, Polydimethylsiloxyethyl Dimethicone/Bis-Vinyldimethicone Crosspolymer, Polydimethylsiloxyethyl Dimethicone/Methicone Copolymer, Polydimethylsiloxy PEG/PPG-24/19 Butyl Ether Silsesquioxane, Polydimethylsiloxy PPG- 13 Butyl Ether Silsesquioxane, Polyglyceryl-3 Disiloxane Dimethicone, Polyglyceryl-3/Lauryl Polydimethylsiloxyethyl Dimethicone Crosspolymer, Polyglyceryl-3 Polydimethylsiloxyethyl Dimethicone, Poly(Glycol Adipate)/Bis-Hydroxyethoxypropyl Dimethicone Copolymer, Polymethylsilsesquioxane, Polymethylsilsesquioxane/Trimethylsiloxysilicate, Polyphenylsilsesquioxane, Polypropylsilsesquioxane, Polysilicone-1, Polysilicone-2, Polysilicone-3, Polysilicone-4, Polysilicone-5, Polysilicone-6, Polysilicone-7, Polysilicone-8, Polysilicone-9, Polysilicone-10, Polysilicone-11, Polysilicone-12, Polysilicone-13, Polysilicone-14, Polysilicone-15, Polysilicone-16, Polysilicone-17, Polysilicone-18, Polysilicone-19, Polysilicone-20, Polysilicone-21, Polysilicone-18 Cetyl Phosphate, Polysilicone-1 Crosspolymer, Polysilicone-18 Stearate, Polyurethane-10, Potassium Dimethicone PEG-7 Panthenyl Phosphate, Potassium Dimethicone PEG- 7 Phosphate, PPG-12 Butyl Ether Dimethicone, PPG-2 Dimethicone, PPG-12 Dimethicone, PPG-27 Dimethicone, PPG-4 Oleth-10 Dimethicone, Propoxytetramethyl Piperidinyl Dimethicone, Propyl Trimethicone, Quaternium-80, Retinoxytrimethylsilane, Silanediol Salicylate, Silanetriol, Silanetriol Arginate, Silanetriol Glutamate, Silanetriol Lysinate, Silanetriol Melaninate, Silanetriol Trehalose Ether, Silica, Silica Dimethicone Silylate, Silica Dimethyl Silylate, Silica Silylate, Silicon Carbide, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, SiliconeQuaternium-16, Silicone Quaternium-16/Glycidoxy Dimethicone Crosspolymer, Silicone Quaternium-17, Silicone Quaternium- 18, Silicone Quaternium-19, Silicone Quaternium-20, Silicone Quaternium-21, Silicone Quaternium- 22, Silicone Quaternium-24, Silicone Quaternium-25, Siloxanetriol Alginate, Siloxanetriol Phytate, Simethicone, Sodium Carboxydecyl PEG-8 Dimethicone, Sodium Dimethicone PEG-7 Acetyl Methyltaurate, Sodium Hyaluronate Dimethylsilanol, Sodium Lactate Methylsilanol, Sodium Mannuronate Methylsilanol, Sodium PCA Methylsilanol, Sodium PG-Propyldimethicone Thiosulfate Copolymer, Sodium PG-Propyl Thiosulfate Dimethicone, Sodium Propoxyhydroxypropyl Thiosulfate Silica, Sorbityl Silanediol, Soy Triethoxysilylpropyldimonium Chloride, Stearalkonium Dimethicone PEG-8 Phthalate, Stearamidopropyl Dimethicone, Steardimonium Hydroxypropyl Panthenyl PEG-7 Dimethicone Phosphate Chloride, Steardimonium Hydroxypropyl PEG-7 Dimethicone Phosphate Chloride, Stearoxy Dimethicone, Stearoxymethicone/Dimethicone Copolymer, Stearoxytrimethylsilane, Stearyl Aminopropyl Methicone, Stearyl Dimethicone, Stearyl/Lauryl Methacrylate Crosspolymer, Stearyl Methicone, Stearyl Triethoxysilanek, Stearyl Trimethicone, Styrene/Acrylates/Dimethicone Acrylate Crosspolymer, Styrene/Acrylates/Dimethicone Copolymer, TEA-Dimethicone PEG-7 Phosphate, Tetrabutoxypropyl Trisiloxane, Tetramethyl Hexaphenyl Tetrasiloxane, Tetramethyl Tetraphenyl Trisiloxane, Tocopheryloxypropyl Trisiloxane, Trideceth-9 PG-Amodimethicone, Triethoxycaprylylsilane, Triethoxysilylethyl Dimethicone/Methicone Copolymer, Triethoxysilylethyl Polydimethylsiloxyethyl Dimethicone, Triethoxysilylethyl Polydimethylsiloxyethyl Hexyl Dimethicone, Triethoxysilylpropylcarbamoyl Ethoxypropyl Butyl Dimethicone, Trifluoromethyl C1-4 Alkyl Dimethicone, Trifluoropropyl Cyclopentasiloxane, Trifluoropropyl Cyclotetrasiloxane, Trifluoropropyl Dimethicone, Trifluoropropyl Dimethicone/PEG-10 Crosspolymer, Trifluoropropyl Dimethicone/Trifluoropropyl Divinyldimethicone Crosspolymer, Trifluoropropyl Dimethicone/Vinyl Trifluoropropyl, Dimethicone/Silsesquioxane Crosspolymer, Trifluoropropyl Dimethiconol, Trifluoropropyldimethyl/trimethylsiloxysilicate, Trifluoropropyl Methicone, Trimethoxycaprylylsilane, Trimethoxysilyl Dimethicone, Trimethyl Pentaphenyl Trisiloxane, Trimethylsiloxyamodimethicone, Trimethylsiloxyphenyl Dimethicone, Trimethylsiloxysilicate, Trimethylsiloxysilicate/Dimethicone Crosspolymer, Trimethylsiloxysilicate/Dimethiconol Crosspolymer, Trimethylsiloxysilylcarbamoyl Pullulan, Trimethylsilyl Hydrolyzed Conchiolin Protein PG-Propyl Methylsilanediol Crosspolymer, Trimethylsilyl Hydrolyzed Silk PG-Propyl Methylsilanediol Crosspolymer, Trimethylsilyl Hydrolyzed Wheat Protein PG-Propyl Methylsilanediol Crosspolymer, Trimethylsilyl Pullulan, Trimethylsilyl Trimethylsiloxy Glycolate, Trimethylsilyl Trimethylsiloxy Lactate, Trimethylsilyl Trimethylsiloxy Salicylate, Triphenyl Trimethicone, Trisiloxane, Tris-Tributoxysiloxymethylsilane, Undecylcrylene Dimethicone, Vinyl Dimethicone, Vinyl Dimethicone/Lauryl Dimethicone Crosspolymer, Vinyl Dimethicone/Methicone Silsesquioxane Crosspolymer, Vinyldimethyl/Trimethylsiloxysilicate Stearyl Dimethicone Crosspolymer, VP/Dimethiconylacrylate/Polycarbamyl/Polyglycol Ester, Zinc Carboxydecyl Trisiloxane and Zinc Dimethicone PEG-8 Succinate, and mixtures thereof.

More preferably the silicones to be contained in the cosmetic or pharmaceutical composition according to the invention are Dimethicone, Cyclomethicone, Cyclopentasiloxane, Cyclotetrasiloxane, Phenyl Trimethicone, and Cyclohexasiloxane.

Waxes and/or stabilizers: Besides natural oils used, one or more waxes may also be present in the cosmetic or pharmaceutical composition according to the present invention, more especially natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Metal salts of fatty acids such as, for example, magnesium, aluminium and/or zinc stearate or ricinoleate may be used as stabilizers.

Primary sun protection factors: In addition to the above disclosed liquid UV filters, the cosmetic or pharmaceutical composition according to the present invention preferably includes one or more further primary sun protection factors in order to optimize the sun protection factor (SPF) of the formulation, i.e. to obtain a high SPF of 5 to 50, or even 5 to 60, and to cover a broad UVA and UVB range. Primary sun protection factors in the context of the invention are, for example, organic substances (light filters) which are liquid or crystalline at room temperature and which are capable of absorbing ultraviolet radiation and of releasing the energy absorbed in the form of longer-wave radiation, for example heat.

The cosmetic or pharmaceutical composition according to the invention advantageously contains at least one UV-A filter and/or at least one UV-B filter and/or a broadband filter and/or at least one inorganic pigment. Compositions according to the invention preferably contain at least one UV-B filter or a broadband filter, more particularly preferably at least one UV-A filter and at least one UV-B filter. Preferred cosmetic compositions, preferably topical compositions according to the present invention comprise one, two, three or more sun protection factors selected from the group consisting of 4-aminobenzoic acid and derivatives, salicylic acid derivatives, benzophenone derivatives, dibenzoylmethane derivatives, diphenyl acrylates, 3-imidazol-4-yl acrylic acid and esters thereof, benzofuran derivatives, benzylidene malonate derivatives, polymeric UV absorbers containing one or more organosilicon radicals, cinnamic acid derivatives, camphor derivatives, trianilino-s-triazine derivatives, 2-hydroxyphenylbenzotriazole derivatives, phenylbenzimidazole sulfonic acid derivatives and salts thereof, anthranilic acid menthyl esters, benzotriazole derivatives and indole derivatives.

In addition, a combination with active ingredients which penetrate into the skin and protect the skin cells from inside against sunlight-induced damage and reduce the level of cutaneous matrix metalloproteases is advantageously. Preferred respective ingredients are so called arylhydrocarbon receptor antagonists. Preferred is 2-benzylidene-5,6-dimethoxy-3,3-dimethylindan-1-one.

The UV filters cited below which can be used within the context of the present invention are preferred but naturally are not limiting.

UV filters which are preferably used are selected from the group consisting of
p-aminobenzoic acid
p-aminobenzoic acid ethyl ester (25 mol) ethoxylated (INCI name: PEG-25 PABA)
p-dimethylaminobenzoic acid-2-ethylhexyl ester
p-aminobenzoic acid ethyl ester (2 mol) N-propoxylated
p-aminobenzoic acid glycerol ester
salicylic acid homomenthyl ester (homosalates) (Neo Heliopan®HMS)
salicylic acid-2-ethylhexyl ester (Neo Heliopan®OS)
triethanolamine salicylate
4-isopropyl benzyl salicylate
anthranilic acid menthyl ester (Neo Heliopan®MA)
diisopropyl cinnamic acid ethyl ester
p-methoxycinnamic acid-2-ethylhexyl ester (Neo Heliopan®AV)
diisopropyl cinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-phenylbenzimidazole sulfonic acid and salts (Neo Heliopan®Hydro)
3-(4′-trimethylammonium) benzylidene bornan-2-one methyl sulfate
beta-imidazole-4(5)-acrylic acid (urocanic acid)
3-(4′-sulfo)benzylidene bornan-2-one and salts
3-(4′-methyl benzylidene)-D,L-camphor (Neo Heliopan®MBC)
3-benzylidene-D,L-camphor
N-[(2 and 4)-[2-(oxoborn-3-ylidene) methyl]benzyl] acrylamide polymer
4,4′-[(6-[4-(1,1-dimethyl)aminocarbonyl) phenylamino]-1,3,5-triazine-2,4-diyl)diimino]-bis-(benzoic acid-2-ethylhexyl ester) (Uvasorb®HEB)
benzylidene malonate polysiloxane (Parsol®SLX)
glyceryl ethylhexanoate dimethoxycinnamate
dipropylene glycol salicylate
tris(2-ethylhexyl)-4,4’,4″-(1,3,5-triazine-2,4,6-triyltriimino)tribenzoate (= 2,4,6-trianilino-(p-carbo-2′-ethylhexyl-1′-oxy)-1,3,5-triazine) (Uvinul®T150).

In a preferred variant the cosmetic or pharmaceutical composition according to the present invention comprises a combination with one or more broadband filters which are selected from the group consisting of
2-ethylhexyl-2-cyano-3,3-diphenyl acrylate (Neo Heliopan®303)
ethyl-2-cyano-3,3′-diphenyl acrylate
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methyl benzophenone
sodium hydroxymethoxybenzophenone sulfonate
disodium-2,2′-dihydroxy-4,4′-dimethoxy-5,5′-disulfobenzophenone
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl®XL)
2,2′-methylene bis-(6-(2H-benzotriazol-2-yl)-4-1,1,3,3-tetramethylbutyl) phenol) (Tinosorb®M)
2,4-bis-[4-(2-ethylhexyloxy)-2-hydroxyphenyl]-1,3,5-triazine
2,4-bis-[{(4-(2-ethylhexyloxy)-2-hydroxy}phenyl]-6-(4-methoxyphenyl)-1,3,5-triazine (Tinosorb®S)
2,4-bis-[{(4-(3-sulfonato)-2-hydroxypropyloxy)-2-hydroxy}phenyl]-6-(4-methoxyphenyl)-1,3,5-triazine sodium salt
2,4-bis-[{(3-(2-propyloxy)-2-hydroxypropyloxy)-2-hydroxy}phenyl]-6-(4-methoxyphenyl)-1,3,5-triazine
2,4-bis-[{4-(2-ethylhexyloxy)-2-hydroxy}phenyl]-6-[4-(2-methoxyethyl carbonyl) phenylamino]-1,3,5-triazine
2,4-bis-[{4-(3-(2-propyloxy)-2-hydroxypropyloxy)-2-hydroxy}phenyl]-6-[4-(2-ethylcarboxyl) phenylamino]-1,3,5-triazine
2,4-bis-[{4-(2-ethylhexyloxy)-2-hydroxy }phenyl]-6-(1-methylpyrrol-2-yl)-1,3,5-triazine
2,4-bis-[{4-tris-(trimethylsiloxysilylpropyloxy)-2-hydroxy}phenyl]-6-(4-methoxyphenyl)-1,3,5-triazine
2,4-bis-[{4-(2″-methylpropenyloxy)-2-hydroxy}phenyl]-6-(4-methoxyphenyl)-1,3,5-triazine
2,4-bis-[{4-(1’,1’,1’,3’,5’,5′,5′-heptamethylsiloxy-2″-methylpropyloxy)-2-hydroxy}phenyl]-6-(4-methoxyphenyl)-1,3,5-triazine.

In a preferred variant the cosmetic or pharmaceutical composition according to the present invention comprises a combination with one or more UV-A filters which are selected from the group consisting of
4-isopropyl dibenzoyl methane
terephthalylidene dibornane sulfonic acid and salts (Mexoryl®SX)
4-t-butyl-4′-methoxydibenzoyl methane (avobenzone) / (Neo Heliopan®357)
phenylene bis-benzimidazyl tetrasulfonic acid disodium salt (Neo Heliopan®AP)
2,2′-(1,4-phenylene)-bis-(1H-benzimidazole-4,6-disulfonic acid), monosodium salt
2-(4-diethylamino-2-hydroxybenzoyl) benzoic acid hexyl ester (Uvinul® A Plus)
indanylidene compounds.

In a more preferred variant, the cosmetic or pharmaceutical composition according to the present invention comprises a combination with one or more UV filters which are selected from the group consisting of
p-aminobenzoic acid
3-(4′-trimethylammonium) benzylidene bornan-2-one methyl sulfate
salicylic acid homomenthyl ester (Neo Heliopan®HMS)
2-hydroxy-4-methoxybenzophenone (Neo Heliopan®BB)
2-phenylbenzimidazole sulfonic acid (Neo Heliopan®Hydro)
terephthalylidene dibornane sulfonic acid and salts (Mexoryl®SX)
4-tert-butyl-4′-methoxydibenzoyl methane (Neo Heliopan®357)
3-(4′-sulfo)benzylidene bornan-2-one and salts
2-ethylhexyl-2-cyano-3,3-diphenyl acrylate (Neo Heliopan®303)
N-[(2 and 4)-[2-(oxoborn-3-ylidene) methyl]benzyl] acrylamide polymer
p-methoxycinnamic acid-2-ethylhexyl ester (Neo Heliopan®AV)
p-aminobenzoic acid ethyl ester (25 mol) ethoxylated (INCI name: PEG-25 PABA)
p-methoxycinnamic acid isoamyl ester (Neo Heliopan®E1000)
2,4,6-trianilino-(p-carbo-2′-ethylhexyl-1′-oxy)-1,3,5-triazine (Uvinul®T150)
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl®XL)
4,4′-[(6-[4-(1,1-dimethyl)aminocarbonyl) phenylamino]-1,3,5-triazine-2,4-diyl)diimino]-bis-(benzoic acid-2-ethylhexyl ester) (Uvasorb HEB)
3-(4′-methyl benzylidene)-D,L-camphor (Neo Heliopan®MBC)
3-benzylidene camphor
salicylic acid-2-ethylhexyl ester (Neo Heliopan®OS)
4-dimethylaminobenzoic acid-2-ethylhexyl ester (Padimate O)
hydroxy-4-methoxybenzophenone-5-sulfonic acid and Na salt
2,2′-methylene bis-(6-(2H-benzotriazol-2-yl)-4-1,1,3,3-tetramethylbutyl) phenol) (Tinosorb®M)
phenylene bis-benzimidazyl tetrasulfonic acid disodium salt (Neo Heliopan®AP)
2,4-bis-[{(4-(2-ethylhexyloxy)-2-hydroxy}phenyl]-6-(4-methoxyphenyl)-1,3,5-triazine (Tinosorb®S)
benzylidene malonate polysiloxane (Parsol®SLX)
menthyl anthranilate (Neo Heliopan®MA)
2-(4-diethylamino-2-hydroxybenzoyl) benzoic acid hexyl ester (Uvinul® A Plus)
indanylidene compounds.

In a further preferred variant according to the present invention the cosmetic or pharmaceutical composition contains a total amount of sunscreen agents, i.e. in particular UV filters and/or inorganic pigments (UV filtering pigments) so that the composition according to the invention has a light protection factor of greater than or equal to 5. Such compositions according to the invention are particularly suitable for protecting the skin and hair.

Secondary sun protection factors: In addition to the above disclosed liquid UV filters and besides the groups of primary sun protection factors mentioned above, secondary sun protection factors of the antioxidant type may also be advantageously used in the cosmetic or pharmaceutical composition according to the present invention. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxyfatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, titanium dioxide (for example dispersions in ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Advantageous inorganic secondary light protection pigments are finely dispersed metal oxides and metal salts. The total quantity of inorganic pigments, in particular hydrophobic inorganic micro-pigments in the finished cosmetic preparation according to the present invention is advantageously from 0.1 to 30% by weight, preferably 0.5 to 10.0% by weight, in each case based on the total weight of the preparation.

Also preferred are particulate UV filters or inorganic pigments, which can optionally be hydrophobed, can be used, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures thereof.

Actives modulating skin and/or hair pigmentation: The cosmetic or pharmaceutical composition according to the present invention may include active ingredients for skin and/or hair lightening. Preferred active ingredients for skin and/or hair lightening are selected from the group consisting of kojic acid (5-hydroxy-2-hydroxymethyl-4-pyranone), kojic acid derivatives, preferably kojic acid dipalmitate, arbutin, ascorbic acid, ascorbic acid derivatives, preferably magnesium ascorbyl phosphate, hydroquinone, hydroquinone derivatives, resorcinol, resorcinol derivatives, preferably 4-alkylresorcinols and 4-(1-phenylethyl)1,3-dihydroxybenzene (phenylethyl resorcinol), cyclohexylcarbamates, sulfur-containing molecules, preferably glutathione or cysteine, alpha-hydroxy acids (preferably citric acid, lactic acid, malic acid), salts and esters thereof, N-acetyl tyrosine and derivatives, undecenoyl phenylalanine, gluconic acid, chromone derivatives, preferably aloesin, flavonoids, 1-aminoethyl phosphinic acid, thiourea derivatives, ellagic acid, nicotinamide (niacinamide), zinc salts, preferably zinc chloride or zinc gluconate, thujaplicin and derivatives, triterpenes, preferably maslinic acid, sterols, preferably ergosterol, benzofuranones, preferably senkyunolide, vinyl guiacol, ethyl guiacol, dionic acids, preferably octodecene dionic acid and/or azelaic acid, inhibitors of nitrogen oxide synthesis, preferably L-nitroarginine and derivatives thereof, 2,7-dinitroindazole or thiocitrulline, metal chelators (preferably alpha-hydroxy fatty acids, phytic acid, humic acid, bile acid, bile extracts, EDTA, EGTA and derivatives thereof), retinoids, soy milk and extract, serine protease inhibitors or lipoic acid or other synthetic or natural active ingredients for skin and hair lightening, the latter preferably used in the form of an extract from plants, preferably bearberry extract, rice extract, papaya extract, turmeric extract, mulberry extract, bengkoang extract, nutgrass extract, liquorice root extract or constituents concentrated or isolated therefrom, preferably glabridin or licochalcone A, artocarpus extract, extract of rumex and ramulus species, extracts of pine species (pinus), extracts of vitis species or stilbene derivatives isolated or concentrated therefrom, saxifrage extract, scutelleria extract, grape extract and/or microalgae extract, in particular Tetraselmis suecica Extract.

Preferred skin lighteners are kojic acid and phenylethyl resorcinol as tyrosinase inhibitors, beta- and alpha-arbutin, hydroquinone, nicotinamide, dioic acid, Mg ascorbyl phosphate and vitamin C and its derivatives, mulberry extract, Bengkoang extract, papaya extract, turmeric extract, nutgrass extract, licorice extract (containing glycyrrhizin), alpha-hydroxy-acids, 4-alkylresorcinols, 4-hydroxyanisole. These skin lighteners are preferred due to their very good activity, in particular in combination with sclareolide according to the present invention. In addition, said preferred skin lighteners are readily available.

Advantageous skin and hair tanning active ingredients in this respect are substrates or substrate analogues of tyrosinase such as L-tyrosine, N-acetyl tyrosine, L-DOPA or L-dihydroxyphenylalanine, xanthine alkaloids such as caffeine, theobromine and theophyl-line and derivatives thereof, proopiomelanocortin peptides such as ACTH, alpha-MSH, peptide analogues thereof and other substances which bind to the melanocortin receptor, peptides such as Val-Gly-Val-Ala-Pro-Gly, -Lys-lle Gly-Arg-Lys or Leu-lle-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, curcumin, zinc diglycinate (Zn(Gly)2), manganese(ll) bicarbonate complexes (“pseudocat-alases”), tetrasubstituted cyclohexene derivatives, isoprenoids, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes to keratinocytes such as serine proteases or agonists of the PAR-2 receptor, extracts of plants and plant parts of the chrysanthemum species, sanguisorba species, walnut extracts, urucum extracts, rhubarb extracts, microalgae extracts, in particular Isochrysis galbana, trehalose, erythrulose and dihydroxyacetone. Flavonoids which bring about skin and hair tinting or browning (e.g. quercetin, rhamnetin, kaempferol, fisetin, genistein, daidzein, chrysin and apigenin, epicatechin, diosmin and diosmetin, morin, quercitrin, naringenin, hesperidin, phloridzin and phloretin) can also be used.

Hair growth activators or inhibitors: Cosmetic or pharmaceutical compositions according to the present invention may also comprise one or more hair growth activators, i.e. agents to stimulate hair growth. Hair growth activators are preferably selected from the group consisting of pyrimidine derivatives such as 2,4-diaminopyrimidine-3-oxide (Aminexil), 2,4-diamino-6-piperidinopyrimidine-3-oxide (Minoxidil) and derivatives thereof, 6-amino-1,2-dihydro-1-hydroxy-2-imino-4-piperidinopyrimidine and its derivatives, xanthine alkaloids such as caffeine, theobromine and theophylline and derivatives thereof, quercetin and derivatives, dihydroquercetin (taxifolin) and derivatives, potassium channel openers, antiandrogenic agents, synthetic or natural 5-reductase inhibitors, nicotinic acid esters such as tocopheryl nicotinate, benzyl nicotinate and C1-C6 alkyl nicotinate, proteins such as for example the tripeptide Lys-Pro-Val, diphencypren, hormones, finasteride, dutasteride, flutamide, bicalutamide, pregnane derivatives, progesterone and its derivatives, cyproterone acetate, spironolactone and other diuretics, calcineurin inhibitors such as FK506 (Tacrolimus, Fujimycin) and its derivatives, Cyclosporin A and derivatives thereof, zinc and zinc salts, polyphenols, procyanidins, proanthocyanidins, phytosterols such as for example beta-sitosterol, biotin, eugenol, (±)-beta-citronellol, panthenol, glycogen for example from mussels, extracts from microorganisms, algae, plants and plant parts of for example the genera dandelion (Leontodon or Taraxacum), Orthosiphon, Vitex, Coffea, Paullinia, Theobroma, Asiasarum, Cucurbita or Styphnolobium, Serenoa repens (saw palmetto), Sophora flavescens, Pygeum africanum, Panicum miliaceum, Cimicifuga racemosa, Glycine max, Eugenia caryophyllata, Cotinus coggygria, Hibiscus rosa-sinensis, Camellia sinensis, Ilex paraguariensis, Isochrysis galbana, licorice, grape, apple, barley or hops or/nd hydrolysates from rice or wheat.

Alternatively, the cosmetic or pharmaceutical composition according to the present invention may include one or more hair growth inhibitors (as described above), i.e. agents to reduce or prevent hair growth. Hair growth inhibitors are preferably selected from the group consisting of activin, activin derivatives or activin agonists, ornithine decarboxylase inhibitors such as alpha-difluoromethylornithine or pentacyclic triterpenes like for example ursolic acid, betulin, betulinic acid, oleanolic acid and derivatives thereof, 5alpha-reductase inhibitors, androgen receptor antagonists, S-adenosylmethionine decarboxylase inhibitors, gamma-glutamyl transpeptidase inhibitors, transglutaminase inhibitors, soybean-derived serine protease inhibitors, extracts from microorganisms, algae, different microalgae or plants and plant parts of for example the families Leguminosae, Solanaceae, Graminae, Asclepiadaceae or Cucurbitaceae, the genera Chondrus, Gloiopeltis, Ceramium, Durvillea, Glycine max, Sanguisorba officinalis, Calendula officinalis, Hamamelis virginiana, Arnica montana, Salix alba, Hypericum perforatum or Gymnema sylvestre.

Enzyme inhibitors: The cosmetic or pharmaceutical composition according to the present invention may comprise one or more enzyme inhibitors. Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen CAT). The substances inhibit enzyme activity, thereby reducing the formation of odour. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Odour absorbers and/or antiperspirant active agents: The cosmetic or pharmaceutical composition according to the present invention may include one or more odour absorbers and/or antiperspirant active agents (antiperspirants). Suitable odour absorbers are substances which are able to absorb and largely retain odour-forming compounds. They lower the partial pressure of the individual components, thus also reducing their rate of diffusion. It is important that perfumes must remain unimpaired in this process. Odour absorbers are not effective against bacteria. They comprise, for example, as main constituent, a complex zinc salt of ricinoleic acid or specific, largely odour-neutral fragrances which are known to the person skilled in the art as “fixatives”, such as, for example, extracts of labdanum or styrax or certain abietic acid derivatives. The odour masking agents are fragrances or perfume oils, which, in addition to their function as odour masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal products, such as, for example, civet and castoreum. Typical synthetic fragrance compounds are products of the ester, ether, aldehyde, ketone, alcohol, and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethole, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden flower oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Suitable astringent antiperspirant active ingredients are primarily salts of aluminium, zirconium or of zinc. Such suitable antihydrotic active ingredients are, for example, aluminium chloride, aluminium chlorohydrate, aluminium dichlorohydrate, aluminium sesquichlorohydrate and complex compounds thereof, e.g. with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, e.g. with amino acids, such as glycine.

Film formers: The cosmetic or pharmaceutical composition according to the present invention may include one or more film formers. Standard film formers are preferably chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Carriers and hydrotropes: The cosmetic or pharmaceutical composition according to the present invention may comprise a carrier or a mixture of different carriers. Preferred cosmetics carrier materials are solid or liquid at 25° C. and 1013 mbar (including highly viscous substances) as for example glycerol, 1,2-propylene glycol, 1,2-butylene glycol, 1,3-propylene glycol, 1,3-butylene glycol, ethanol, water, and mixtures of two or more of said liquid carrier materials with water. Optionally, these preparations according to the invention may be produced using preservatives or solubilizers. Other preferred liquid carrier substances, which may be a component of a preparation according to the invention are selected from the group consisting of oils such as vegetable oil, neutral oil and mineral oil.

Preferred solid carrier materials, which may be a component of the cosmetic or pharmaceutical composition according to the invention are hydrocolloids, such as starches, degraded starches, chemically or physically modified starches, dextrins, (powdery) maltodextrins (preferably with a dextrose equivalent value of 5 to 25, preferably of 10 - 20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin, inulin, and mixtures of two or more of these solids, in particular maltodextrins (preferably with a dextrose equivalent value of 15 - 20), lactose, silicon dioxide and/or glucose.

In addition, hydrotropes, for example ethanol, isopropyl alcohol or polyols, may be used to improve flow behaviour. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 Dalton;
technical oligoglycerol mixtures with a degree of self-condensation of 1.5 to 10, such as for example technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Dyes: The cosmetic or pharmaceutical composition according to the present invention may comprise one or more dyes. Suitable dyes are any of the substances suitable and approved for cosmetic purposes. Examples include cochineal red A (CI. 16255), patent blue V (Cl. 42051), indigotin (Cl. 73015), chlorophyllin (Cl. 75810), quinoline yellow (Cl. 47005), titanium dioxide (Cl. 77891), indanthrene blue RS (CI. 69800) and madder lake (Cl. 58000). Luminol may also be present as a luminescent dye. Advantageous coloured pigments are for example titanium dioxide, mica, iron oxides (e.g. Fe₂O₃ Fe3O₄, FeO(OH)) and/or tin oxide. Advantageous dyes are for example carmine, Berlin blue, chromium oxide green, ultramarine blue and/or manganese violet.

In addition to the above-described substances, further ingredients commonly used in the cosmetic or pharmaceutical industry, which are suitable or customary in the compositions of the present invention, can be used.

The 1,2-heptanediol or the 2,3-heptanediol or the mixture comprising 1,2-heptanediol and 2,3-heptanediol according to the first aspect of the present invention, in an amount of 0.001% to 15.0 by weight, based on the total weight of the composition. In a preferred variant, the cosmetic or pharmaceutical composition comprises the 1,2-heptanediol or the 2,3-heptanediol or the mixture comprising 1,2-heptanediol and 2,3-heptanediol in an amount of 0.01 to 10.0% by weight, based on the total weight of the composition. In a more preferred variant, the 1,2-heptanediol or the 2,3-heptanediol or the mixture comprising 1,2-heptanediol and 2,3-heptanediol is advantageously used in the cosmetic or pharmaceutical composition in an amount of at 0.1 to 5.0% by weight, based on the total weight of the composition. In a still more preferred variant, the 1,2-heptanediol or the 2,3-heptanediol or the mixture comprising 1,2-heptanediol and 2,3-heptanediol is advantageously used in the cosmetic or pharmaceutical composition in an amount of at 0.3 to 3.0% by weight, based on the total weight of the composition. In a most preferred variant, the 1,2-heptanediol or 2,3-heptanediol or the mixture comprising 1,2-heptanediol and 2,3-heptanediol is advantageously used in the cosmetic or pharmaceutical composition in an amount of at 0.5 to 1.0% by weight, based on the total weight of the composition.

For the heptanediol mixture, the above amounts relate to the total content of the 1,2-heptanediol and the 2,3-heptanediol in the mixture, i.e. the amount is the sum of the content of alkanediolsalkanediols1,2-heptanediol and 2,3-heptanediol in the mixture.

The at least one linear alkanediol or the mixture comprising at least one first linear alkanediol and one or more second linear alkanediol according to the second aspect of the present invention is present in the cosmetic or pharmaceutical composition in an amount of 0.001 to 15.0% by weight, based on the total weight of the composition. In a preferred variant, the cosmetic or pharmaceutical composition comprises the at least one linear alkanediol or the mixture comprising at least one first linear alkanediol and one or more second linear alkanediol in an amount of 0.01 to 10.0% by weight, based on the total weight of the composition. In a more preferred variant, the at least one linear alkanediol or the mixture comprising at least one first linear alkanediol and one or more second linear alkanediol is advantageously used in the cosmetic or pharmaceutical composition in an amount of at 0.1 to 5.0% by weight, based on the total weight of the composition. In a still more preferred variant, the at least one linear alkanediol or the mixture comprising at least one first linear alkanediol and one or more second linear alkanediol is advantageously used in the cosmetic or pharmaceutical composition in an amount of at 0.3 to 3.0% by weight, based on the total weight of the composition. In a most preferred variant, the at least one linear alkanediol or the mixture comprising at least one first linear alkanediol and one or more second linear alkanediol is advantageously used in the cosmetic or pharmaceutical composition in an amount of at 0.5 to 1.0% by weight, based on the total weight of the composition.

For the alkane mixture, the above amounts relate to the total content of the first linear alkanediol and the second linear alkanediol in the mixture, i.e. the amount is the sum of the content of the first linear alkanediol and the second linear alkanediol in the mixture.

In a particular preferred variant according to the first aspect of the present invention, the cosmetic or pharmaceutical composition comprises the 2,3-heptanediol or the 2,3-heptanediol of the mixture comprising 1,2-heptanediol and 2,3-heptanediol in an amount of 0.001 to 15.0% by weight, preferred in an amount of 0.01 to 10.0% by weight, more preferred in an amount of 0.1 to 5.0% by weight, still more preferred in an amount of 0.3 to 3.0% by weight, and most preferred in an amount of 0.5 to 1.0% by weight, based on the total weight of the cosmetic or pharmaceutical composition.

Even more preferred, the cosmetic or pharmaceutical composition comprises the 2,3-heptanediol or the 2,3-heptanediol of the mixture comprising 1,2-heptanediol and 2,3-heptanediol in an amount of 0.001 to 0.5% weight, preferably in an amount of 0.005 to 0.1% by weight and most preferably in an amount of 0.01 to 0.075% by weight, based on the total weight of the composition.

In a particular preferred variant according to the first aspect of the present invention, the cosmetic or pharmaceutical composition comprises the 2,3-alkanediol as linear alkanediol or of the mixture comprising at least one first linear alkanediol and one or more second linear alkanediol in an amount of 0.001 to 15.0% by weight, preferably in an amount of 0.01 to 10.0% by weight, more preferred in an amount of 0.1 to 5.0% by weight, still more preferred in an amount of 0.3 to 3.0% by weight, and most preferred in an amount of 0.5 to 1.0% by weight, based on the total weight of the cosmetic or pharmaceutical composition.

Even more preferred, the cosmetic or pharmaceutical composition comprises the 2,3-alkanediol as linear alkanediol or of the mixture comprising at least one first linear alkanediol and one or more second linear alkanediol in an amount of 0.001 to 0.5% weight, preferably in an amount of 0.005 to 0.1% by weight and most preferably in an amount of 0.01 to 0.075% by weight, based on the total weight of the cosmetic or pharmaceutical composition.

If the cosmetic or pharmaceutical composition according to the present invention is an emulsion, the liquid lipophilic component is present in the cosmetic or pharmaceutical composition in an amount of 0.01 to 50.0% by weight, based on the total weight of the composition. In a preferred variant, the cosmetic or pharmaceutical composition comprises the liquid lipophilic component in an amount of 1.0 to 40.0 % by weight, based on the total weight of the composition. In a particular preferred variant, the liquid lipophilic component is advantageously used in the cosmetic or pharmaceutical composition in an amount of at 3.0 to 30% by weight, based on the total weight of the composition.

If the cosmetic or pharmaceutical composition according to the present invention is a water free formulation, i.e. an oil formulation, the liquid lipophilic component is present in the cosmetic or pharmaceutical composition in an amount of ≥ 60% by weight, preferably in an amount of ≥ 75% by weight, more preferably in an amount of ≥ 90% by weight, based on the total weight of the composition.

If the cosmetic or pharmaceutical composition according to the present invention is a solid surfactant composition, the liquid lipophilic component is present in the cosmetic or pharmaceutical composition in an amount of 1 to 40% by weight, preferably in an amount of 3 to 30% by weight, more preferably in an amount of 5 to 25% by weight, based on the total weight of the composition.

The cosmetic or pharmaceutical composition according to the first aspect or the second aspect of the present invention is further characterized in that it does not contain a silicone or silicone derivative, in particular a silicone oil.

In a preferred variant, the cosmetic or pharmaceutical composition does not contain one or more silicone or silicone derivative as defined and specified in detail above.

Surprisingly, it was found, as described in more detail in the following Example 1, that 1,2-heptanediol is easier to incorporate into a liquid lipophilic component or the cosmetic or pharmaceutical composition as described in the present invention. The solubility of the 1,2-heptanediol in a liquid lipophilic containing formulation or composition is superior compared to the solubility of 1,2-pentanediol and 1,2-hexanediol, as is demonstrated by the following examples. In contrast, 1,2-octanediol tends to precipitate or recrystallize in oily solutions. Due to their excellent solubility in a liquid lipophilic component containing composition, the 1,2-heptanediol can be advantageously provided in high concentrations in the composition according to the present invention. This in turn enhances the availability of the 1,2-heptanediol in the topical formulation or composition.

Surprisingly, it was found, as described in more detail in the following Example 1, that 2,3-alkanediols, in particular 2,3-heptanediol or 2,3-octanediol, is/are easier to incorporate into a liquid lipophilic component or the cosmetic or pharmaceutical composition as described in the present invention. Unlike 1,2-octanediol, the solubility of the 2,3-octanediol in a liquid lipophilic containing formulation or composition is superior and results in a clear, non-turbid solution Also the solubility of a combination of a 1,2-alkanediol and a 2,3-alkanediol, in particular 1,2-octanediolwith 2,3-octanediol and 1,2-heptanediol with 2,3-heptanediol, is excellent and results in a clear and non-turbid solution. The use of 2,3-octanediol in mixture with 1,2-octanediol, even in small amounts, can improve the solubility of 1,2-octanediol, which alone tends to precipitate or recrystallize in oily solutions. Due to their excellent lipid phase solubility, the 2,3-alkanediols can be advantageously provided in high concentrations in the composition according to the present invention. This in turn enhances the availability of the 2,3-alkanediols in the topical formulation or composition.

As is further demonstrated in the following examples, the presence of 1,2-heptanediol or a linear alkanediol or a mixture of at least one first linear alkanediol with one or more second linear alkanediol improves the skin sensation of a cosmetic or pharmaceutical composition comprising at least one liquid lipophilic component upon topical application: The combination of a liquid lipophilic component as defined above with 1,2-heptanediol or a linear alkanediol or a mixture of at least one first linear alkanediol with one or more second linear alkanediol surprisingly results in an improved spreading behavior or reduces the fatty/greasy skin feel of oily ingredients. In parallel, the absorption (less remaining residue) was improved. These attributes are favourable for the application of topical formulations or compositions.

As can be seen from Tables 2 to 21, the combined use of 1,2-heptanediol and a liquid lipophilic ingredient significantly improves the spreading behavior and/or significantly reduces the fatty/greasy skin feel upon application of the composition on the skin. In parallel, the absorption (less remaining residue) was improved too.

As it is further demonstrated by the Examples specified in Table22, with the use of 1,2-heptanediol or 2,3-heptanediol or a mixture of 1,2-heptanediol and 2,3-heptanediol or the use of 1,2-octanediol or 2,3 octanediol or a mixture of 1,2-octanediol and 2,3-octanediol the spreading behavior of liquid lipophilic components can be significantly improved. Additionally, for all samples an oily or fatty/greasy skin feeling, imparting unwanted gloss to the skin and rendering the composition sticky, can be considerably improved by the use of one of the aforementioned alkanediols. Furthermore, the test shows, that the liquid lipophilic ingredients of the composition are better absorbed and do not leave a discomfort on the skin when applied in combination with one of the afore mentioned alkanediol.

For some liquid lipophilic component samples even two or three of the above mentioned parameters, i.e. spreading and/or fatty/greasy skin feeling and/or absorption can be improved, resulting in excellent sensory properties of the applied cosmetic or pharmaceutical composition comprising a liquid lipophilic component according to the present invention.

As it is obvious from Table 22, a mixture including 1,2-heptanediol and 2,3-heptanediol improves at least one parameter “spreading”, “fatty/greasy skin” and/or “absorption/remaining residue” toward plant oils such as argan oil, avocado oil, castor oil, grapeseed oil, upon topical application in comparison to the use of the single alkanediol substances. A mixture including 1,2-heptanediol and 2,3-heptanediol improves at least two parameters “spreading”, “fatty/greasy skin” and/or “absorption/remaining residue” toward plant oils such as cacao butter, CCT (neutral oil), olive oil, upon topical application in comparison to the single alkanediol substances. The same effect could be observed for the heptanediol mixture with respect to Octocrylene (Neo Heliopan 303); spreading and absorption were improved in comparison to the use of the alkanediol substances alone.

A mixture including 1,2-octanediol and 2,3-octanediol improves at least two or even three parameters “spreading”, “fatty/greasy skin” and/or “absorption/remaining residue” toward plant oils such argan oil, avocado oil, castor oil, olive oil, or with respect to Octorylene (Neo Heliopan 303) in comparison to 1,2-octanediol or 2,3-octanediol alone. This effect makes said alkanediol mixture particularly beneficial.

Also, a mixture including 1,2-hexanediol and 2,3-hexanediol improves at least two parameters as defined above toward plant oils such as CCT (neutral oil).

Compared to the single 1,2-alkanediol compounds, also the corresponding 2,3-alkanediol compounds such as 2,3-heptanediol or 2,3-octanediol showed an improvement in at least one, advantageously two parameters “spreading”, fatty/greasy skin feel” and/or absorption which effect make the 2,3-alkanediol compounds as defined herein particular valuable for the improvement of the sensory properties of liquid lipophilic components.

In particular, liquid lipophilic components which have a poor spreadability by nature, which initial spreading, i.e. without the addition of an alkanediol components, was scored with < 2, show a remarkably improvement in spreading both the tested single alkanediol compounds and the tested alkanediol mixture. 1,2-heptanediol and a mixture of 1,2-heptanediol and 2,3-heptanediol perform best.

Surprisingly, the cosmetic or pharmaceutical composition according to the present invention has a similar spreading behaviour and significantly reduces the fatty/greasy skin feel of oily ingredients compared to a cosmetic or pharmaceutical oil composition comprising the same amount silicon, as it is demonstrated by Example 5. The tested sunflower oil compositions have almost the same absorption (less remaining residue). Hence, the use of alkanediols as defined before, in particular 1,2-heptanediol, are/is suitable to partially or completely replace a silicone in a cosmetic or pharmaceutical composition, in order to achieve a superior skin sensation as usually achieved by the addition of silicone. By the complete or partial replacement of silicone with a green alkanediol in a cosmetic or pharmaceutical composition, the naturality index, which is the criteria for natural and organic cosmetic ingredients, of a cosmetic or pharmaceutical formulation, can be significantly increased.

The cosmetic or pharmaceutical, preferably dermatological, composition according to the first aspect or second aspect of the present invention is intended for topical applications. The term “topical” is understood to mean external applications on a mammal’s skin, which are in particular for the treatment, protection, care and cleansing of the skin, scalp, eyelashes, eyebrows, nails, mucous membranes and hair. The mammal is preferably a human.

For topical application, the cosmetic or pharmaceutical composition is either a rinse off or leave on preparation.

The cosmetic or pharmaceutical, in particular dermatological, composition according to the first aspect and second aspect of the present invention can be present in different forms of, e.g. in the form of a dispersion, in the form of a water free formulation, in the form of a solid surfactant formulation, or in the form of an aqueous, aqueous/alcoholic, in particular aqueous/ethanolic, or an aqueous/glycolic based solution.

In a particular variant, the cosmetic or pharmaceutical composition according to the present invention is a cold formulation, which can be prepared without heating step.

In a preferred variant, the cosmetic or pharmaceutical, preferably dermatological, composition according to the first aspect and second aspect of the present invention is a dispersion. The term “dispersion” in the context of the present invention means, that the cosmetic or pharmaceutical composition is a disperse two-phase system consisting of colloidal particles (disperse phase) and a medium in which they are suspended (disperse medium). Both phases are not miscible with each other, only with an emulsifier. Such dispersions, for example emulsions, comprise the at least one liquid lipophilic component preferably in an amount of ≥ 1% by weight, more preferably in an amount of ≥ 3% by weight.

Preferably, the cosmetic or pharmaceutical composition according to the first aspect or second aspect of the present invention takes various forms such as an emulsion, in particular a O/W emulsion, a W/O emulsion, a multiple emulsion, a hydrodispersion gel, a balm, a multiple emulsion of the water-in-oil type (W/O/WO) or of the oil-in-water type (O/W/O), PIT emulsion, Pickering emulsion, a micro-emulsion, a liquid, a lotion, a suspension, a milk, an ointment, a paste, a gel, a cream based, an oil based or a liposome based formulation.

In a further variant, the cosmetic or pharmaceutical, preferably dermatological, composition according to the first aspect and second aspect of the present invention is a water free formulation, i.e. an oil formulation. The term water free formulation in the context of the present invention means that the at least one liquid lipophilic component is present in an amount of ≥ 60% by weight, preferably in an amount of ≥ 90% by weight.

Such water free formulations include e.g. oils, skin butters, powders, lip stick, antiperspirant/deo stick, and decorative cosmetics, etc.

In a further variant, the cosmetic or pharmaceutical, preferably dermatological, composition according to the first aspect and second aspect of the present invention is a solid surfactant composition.

Such solid surfactant compositions include e.g. solid shampoos, solid body wash, bar soaps.

Alternatively, the cosmetic or pharmaceutical composition as disclosed herein is an aqueous or aqueous/alcoholic, preferably aqueous/ethanolic, or aqueous/glycolic based solution. The aqueous/alcoholic or aqueous/glycolic based solution comprises an aliphatic alcohol or a glycol in an amount of 0.1 to ≤ 50% by weight, based on the total weight of the solution. The aliphatic alcohol is preferably selected from the group consisting of ethanol, isopropanol, n-propanol. The glycol is preferably selected from the group consisting of glycerin, propylene glycol, butylene glycol or dipropylene glycol. Preferably, the overall water content in the final composition of such compositions can be ≥ 60%, more preferably ≥ 70%, even more preferably ≥ 80 %, and even more preferably ≥ 90%. New applications such as those including solutions for water wipes have high water content. In a particular variant, the inventive compositions can be used for such wet wipe applications. They may then most preferably contain ≥ 95 % water, or even ≥ 98% water.

The higher the water content in such a formulation, the lower the solubility of the liquid lipophilic components.

Such aqueous or aqueous/alcoholic or aqueous/glycolic based solutions include for example deo/antiperspirant preparations, after shave, cleansing preparations, anti-acne preparations, or wet wipe solutions.

In one preferred variant, the cosmetic or pharmaceutical composition according to the present invention is an emulsion as defined herein, advantageously an oil-in-water (O/W) emulsion comprising an oily phase dispersed in an aqueous phase in the presence of an O/W emulsifier or is a water free formulation or a solid surfactant composition.

These formulations or compositions are prepared according to usual and known methods.

Preferably, the cosmetic composition according to the present invention is a preparation for cosmetic use and/or non-therapeutic use in personal care, preferably for skin protection, skin care, scalp protection, scalp care, hair care, nail care, in particular for the prevention and/or treatment of skin conditions, intolerant or sensitive skin, skin irritation, skin reddening, rosacea, wheals, pruritus (itching), skin aging, wrinkle formation, loss of skin volume, loss of skin elasticity, pigment spots, pigment abnormalities, skin dryness, flaking, greasiness, hypopigmentation and/or hyperpigmentation of the skin; or is a functional preparation or a preparation for animal care.

Examples of personal care are preferably anti-ageing preparations, skin care emulsions, body oils, body lotions, cleansing lotions, face or body balms, after shave balms, after sun balms, deo emulsions, cationic emulsions, body gels, treatment creams, skin protection ointments, moisturizing gels, face and/or body moisturizers, light protective preparations (sunscreens), micellar water, hair spray, colour protection hair care products, skin lightening product, anti-dark spot preparations, etc.

Alternatively, the composition according to the present invention is a preparation for medical use.

The pharmaceutical, in particular dermatological, composition according to the present invention is a preparation for the prevention and treatment of a condition of the skin or mucosa.

In order to be used, the cosmetic or pharmaceutical, in particular dermatological composition according to the first aspect or second aspect of the present invention is applied to the skin, hair, scalp and/or nails in an adequate amount in such manner as is customary with cosmetics and dermatological products.

Surprisingly, 1,2-heptanediol or 2,3-heptanediol or a mixture comprising 1,2-heptanediol and 2,3-heptanediol or the at least one linear alkanediol or the mixture of at least one first linear alkanediol with one or more second linear alkanediol, in particular 2,3-alkanediols, have a significantly better solubility in the liquid lipophilic component containing composition compared to 1,2-pentanediol or 1,2-hexanediol, which lead to turbid solutions, or 1,2-octanediol, which tends to precipitate or recrystallize in oily solutions.

Additionally, said 1,2-heptanediol and/or 2,3-heptanediol or 2,3-alkanediols do not precipitate in the liquid lipophilic component containing composition and, thus, do not lead to turbidity. Thus, due to their better solubility stable liquid lipophilic component containing composition can be obtained and even maintained during storage.

It was found that 1,2-heptanediol or 2,3-heptanediol or a mixture including 1,2-heptanediol and 2,3-heptanediol or the at least one linear alkanediol or the mixture of at least one first linear alkanediol with one or more second linear alkanediol, in particular 2,3-alkanediols, used according to the present invention are available in the liquid lipophilic component containing composition in a high concentration due to its better solubility. This resulted in an improved availability for the liquid lipophilic component, but also provided synergistic effects for the combination with the alkanediol.

Surprisingly, the use of 1,2-heptanediol or 2,3-heptanediol or a mixture including 1,2-heptanediol and 2,3-heptanediol or the at least one linear alkanediol or the mixture of at least one first linear alkanediol with one or more second linear alkanediol in a liquid lipophilic component containing cosmetic or pharmaceutical composition according to the first aspect or second aspect of the present invention improves the skin sensation. Upon topical application, the use of 1,2-heptanediol or the at least one linear alkanediol or the mixture of at least one first linear alkanediol with one or more second linear alkanediol in a liquid lipophilic component containing cosmetic or pharmaceutical composition leads to improved sensory properties upon application, as it is demonstrated by the following examples.

Thus, the present invention relates in a further aspect to the use of 1,2-heptanediol or 2,3-heptanediol or a mixture including 1,2-alkanediol and 2,3-alkanediol or the at least one linear alkanediol or the mixture of at least one first linear alkanediol with one or more second linear alkanediol for improving sensory properties such as skin sensation of a cosmetic or pharmaceutical composition comprising at least one liquid lipophilic component upon topical application. In particular, advantageous sensory properties are obtained by improving the spreading behaviour of the composition, reducing an oily, greasy feeling and unwanted gloss. Furthermore, the composition is better absorbed and do not leave a discomfort of the absorbed product when applied on the skin.

Surprisingly, the use of 1,2-heptanediol and/or 2,3-heptanediol or the at least one linear alkanediol or the mixture of at least one first linear alkanediol with one or more second linear alkanediol in a liquid lipophilic component containing cosmetic or pharmaceutical composition according to the first aspect or second aspect of the present invention leads to an almost identical skin sensation than a liquid lipophilic component containing cosmetic or pharmaceutical composition comprising instead the same amount of a silicone.

Thus, the present invention relates in a further aspect to the use of 1,2-heptanediol and/or 2,3-heptanediol or the at least one linear alkanediol or the mixture of at least one first linear alkanediol with one or more second linear alkanediol as a silicone replacer in a cosmetic or pharmaceutical composition comprising at least one liquid lipophilic component.

Additionally, in a further aspect, the present invention relates to the use of 1,2-heptanediol and/or 2,3-heptanediol or the at least one first linear alkanediol or the mixture of at least one first linear alkanediol with one or more second linear alkanediol for preparing a cosmetic or pharmaceutical composition comprising at least one liquid lipophilic component upon topical application.

The present invention shall now be described in detail with reference to the following examples, which are merely illustrative of the present invention, such that the content of the present invention is not limited by or to the following examples.

### EXAMPLES

### Example 1: Solubility of Different Alkanediols or Mixtures Thereof in Oil

Each 15 g 1,2-pentanediol, 1,2-hexanediol, 1,2-heptanediol or 1,2-octanediol were dissolved in 85 g sunflower oil. Each 15 g of a mixture of 1,2-octanediol and 2,3-octanediol (95 : 5) or 1,2-heptanediol and 2,3-heptanediol (95 : 5) were dissolved in 85 g sunflower oil.

The solubility of the different alkanediols or mixtures thereof is shown in FIG. 1: 1,2 pentanediol and 1,2 hexanediol are well-known cosmetic ingredients, but their solubility in oils is poor in comparison to 1,2 heptanediol. 1,2 heptanediol showed a better solubility in oily ingredients. 1,2-octanediol tends to precipitate or recrystallize in sunflower oil. Unlike 1,2-octanediol, 2,3-octanediol has surprisingly a good solubility in sunflower oil. Also, mixtures of 1,2-octanediol and 2,3-octanediol or 1,2-heptanediol and 2,3-heptanediol surprisingly have a good solubility and lead to clear solutions.

### Example 2: Sensory Properties of Topical Compositions According to the Present Invention

For evaluating the sensory behaviour of 1,2-heptanediol in combination with different liquid lipophilic components, oil components specified in the following Table 1 were blended with 1,2-heptanediol (formulation) in a ratio as specified in the tables and assessed by untrained panellists in comparison to the liquid lipophilic component without addition of 1,2-heptanediol (placebo).

Test procedure:
Hands were pre-washed with liquid soap by following a defined procedure;
30 µl of corresponding sample was applied on the backside of each hand (randomized);
After 30 s the spreading behaviour of the respective formulation on skin was evaluated versus the respective oil component without 1,2-heptanediol on a scale from 1 to 5 (1 = low spreading; 5 = high spreading);
The samples were distributed with the fingertips of middle and forefinger with 2 x 5 cycles;
After 2 minutes, the fatty/greasy skin feel was evaluated on a scale from 1 to 5 (1 = low fatty/greasy skin feel; 5 = high fatty/greasy skin feel);
Furthermore the applicants were asked to assess the absorption, i.e. the impression of amount of remaining residue on the skin on a scale from 1 to 5 (1 = low absorption; 5 = high absorption).

TABLE 1

Tested ingredients
INCl
Caprylic Capric Triglyceride (CCT) (neutral oil)
Helianthus Annuus (Sunflower) Seed Oil
Butyrospermum Parkii (Shea) Butter
Propanediol Dicaprylate/Dicaprate (Symollient PDCC)
Prunus Amygdalus Dulcis (Sweet Almond) Oil
Simmondsia Chinensis (Jojoba) Seed Oil
Cetearyl Nonanoate (SymMollient S)
Hexyldecanol (Eutanol G)
Octocrylene (Neo Heliopan 303)
Ethylhexyl Salicylate (Neo Heliopan OS)
Ethylhexyl Methoxycinnamate (Neo Heliopan AV)
Isoamyl p-Methoxycinnamate (Neo Heliopan E 1000)
Homosalate (Neo Heliopan HMS)
Chlorella oil (biotech)
Microalgae oil

### Results

TABLE 2

Caprylic Capric Triglyceride
Caprylic Capric Triglyceride + 1,2 heptanediol; 85 : 15 w/w % (B, B1; B2) Caprylic Capric Triglyceride, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 4 | 5 | 2 | 4 | 3 |
| 2 | 1 | 2 | 2 | 3 | 2 | 3 |
| 3 | 1 | 4 | 3 | 2 | 3 | 2 |
| 4 | 1 | 4 | 5 | 3 | 4 | 2 |
| 5 | 3 | 2 | 5 | 3 | 4 | 3 |
| 6 | 1 | 3 | 2 | 3 | 4 | 4 |
| 7 | 1 | 5 | 4 | 3 | 4 | 2 |
| 8 | 1 | 3 | 4 | 2 | 4 | 3 |
| 9 | 1 | 4 | 2 | 4 | 4 | 3 |
| 10 | 1 | 4 | 1 | 2 | 2 | 3 |
| 11 | 3 | 4 | 2 | 3 | 3 | 3 |
| 12 | 2 | 4 | 4 | 2 | 2 | 1 |
| 13 | 3 | 2 | 4 | 5 | 4 | 3 |
| 14 | 5 | 3 | 2 | 4 | 2 | 3 |
| mean value | 1.9 | 3.4 | 3.2 | 2.9 | 3.3 | 2.7 |

TABLE 3

Sunflower oil
Sunflower Oil + 1,2 heptanediol; 85 : 15 w/w % (B, B1, B2) Sunflower Oil, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 1 | 4 | 4 | 2 | 4 | 1 |

TABLE 4

Shea butter (Cetiol SB45)
Shea butter + 1,2 heptanediol; 50 : 50 w/w % (B, B1, B2) Shea butter, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 4 | 5 | 2 | 5 | 4 |
| 2 | 2 | 4 | 4 | 2 | 3 | 2 |
| 3 | 3 | 3 | 4 | 2 | 3 | 1 |
| 4 | 1 | 4 | 4 | 2 | 5 | 3 |
| 5 | 3 | 5 | 3 | 2 | 5 | 4 |
| 6 | 1 | 4 | 4 | 2 | 4 | 2 |
| mean value | 2.0 | 4.0 | 4.0 | 2.0 | 4.2 | 2.7 |

TABLE 5

Shea butter/Caprylic Capric Triglyceride
Shea butter + Caprylic Capric Triglyceride + 1,2 heptanediol, ratio 1:1:1 (B, B1, B2) Shea butter + Caprylic Capric Triglyceride, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 3 | 4 | 1 | 5 | 3 |
| 2 | 1 | 3 | 4 | 3 | 3 | 4 |
| 3 | 2 | 4 | 3 | 1 | 4 | 2 |
| 4 | 2 | 5 | 3 | 1 | 2 | 2 |
| 5 | 1 | 4 | 4 | 3 | 4 | 3 |
| 6 | 2 | 4 | 4 | 2 | 4 | 1 |
| mean value | 1.7 | 3.8 | 3.7 | 2.3 | 3.7 | 2.5 |

TABLE 6

Propanediol Dicaprylate/Caprate
Propanediol Dicaprylate/Caprate + 1,2 heptanediol; 85 : 15 w/w % (B, B1, B2) Propanediol Dicaprylate/Caprate, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 3 | 4 | 1 | 4 | 2 |
| 2 | 1 | 2 | 3 | 2 | 4 | 1 |
| 3 | 3 | 2 | 1 | 3 | 2 | 3 |
| 4 | 1 | 3 | 3 | 4 | 3 | 4 |
| 5 | 1 | 3 | 3 | 2 | 2 | 3 |
| 6 | 2 | 4 | 3 | 1 | 2 | 1 |
| 7 | 2 | 3 | 2 | 3 | 1 | 3 |
| 8 | 2 | 3 | 1 | 2 | 2 | 1 |
| 9 | 1 | 2 | 4 | 1 | 4 | 1 |
| 10 | 1 | 3 | 3 | 4 | 2 | 4 |
| 11 | 1 | 3 | 2 | 3 | 3 | 3 |
| 12 | 2 | 3 | 3 | 5 | 3 | 2 |
| 13 | 2 | 4 | 3 | 2 | 1 | 3 |
| mean value | 1.6 | 2.9 | 2.7 | 2.5 | 2.5 | 2.4 |

TABLE 7

Sweet Almond oil
Sweet Almond Oil + 1,2 heptanediol; 85 : 15 w/w % (B, B1, B2) Sweet Almond Oil, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 1 | 3 | 4 | 3 | 4 | 2 |
| 2 | 1 | 3 | 4 | 2 | 4 | 2 |
| 3 | 2 | 3 | 1 | 3 | 3 | 3 |
| 4 | 2 | 4 | 4 | 3 | 4 | 2 |
| 5 | 1 | 2 | 3 | 1 | 3 | 1 |
| 6 | 1 | 3 | 4 | 3 | 4 | 3 |
| mean value | 1.3 | 3.0 | 3.3 | 2.5 | 3.7 | 2.2 |

TABLE 8

Jojoba oil
Jojoba Oil + 1,2 heptanediol; 85 : 15 w/w % (B, B1, B2) Jojoba Oil, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 4 | 4 | 2 | 4 | 1 |

TABLE 9

Cetearyl Nonanoate
Cetearyl Nonanoate + 1,2 heptanediol, 85 : 15 w/w % (B, B1, B2) Cetearyl Nonanoate, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 3 | 4 | 3 | 4 | 2 |
| 2 | 1 | 5 | 2 | 1 | 3 | 2 |
| 3 | 2 | 5 | 3 | 2 | 4 | 2 |
| 4 | 2 | 5 | 4 | 3 | 3 | 1 |
| 5 | 1 | 3 | 2 | 4 | 2 | 3 |
| 6 | 2 | 4 | 4 | 2 | 3 | 2 |
| mean value | 1.7 | 4.2 | 3.2 | 2.5 | 3.2 | 2.0 |

TABLE 10

Hexyldecanol
Hexyldecanol + 1,2 heptanediol, 85 : 15 w/w % (B, B1, B2) Hexyldecanol, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 3 | 4 | 3 | 4 | 2 | 3 |
| 2 | 2 | 5 | 3 | 1 | 3 | 1 |
| 3 | 2 | 4 | 2 | 2 | 2 | 3 |
| 4 | 4 | 2 | 4 | 3 | 4 | 3 |
| 5 | 2 | 3 | 3 | 4 | 2 | 3 |
| 6 | 2 | 3 | 3 | 4 | 2 | 3 |
| mean value | 2.5 | 3.5 | 3.0 | 3.0 | 2.5 | 2.7 |

TABLE 11

Octocrylene
Octocrylene + 1,2 heptanediol, 85 : 15 w/w % (B, B1, B2) Octocrylene, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 1 | 2 | 3 | 5 | 5 | 4 |
| 2 | 1 | 4 | 3 | 4 | 5 | 4 |
| 3 | 1 | 2 | 2 | 3 | 3 | 2 |
| 4 | 1 | 4 | 1 | 3 | 4 | 1 |
| 5 | 1 | 2 | 1 | 3 | 3 | 2 |
| 6 | 1 | 2 | 1 | 2 | 4 | 3 |
| mean value | 1.0 | 2.7 | 1.8 | 3.3 | 4.0 | 2.7 |

TABLE 12

Ethylhexyl Salicylate
Ethylhexyl Salicylate + 1,2 heptanediol, 85 : 15 w/w % (B, B1, B2) Ethylhexyl Salicylate, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 1 | 4 | 3 | 2 | 2 | 2 |

TABLE 13

Ethylhexyl Methoxycinnamate
Ethylhexyl Methoxycinnamate + 1,2 heptanediol, 85 : 15 w/w % (B, B1, B2) Ethylhexyl Methoxycinnamate, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 4 | 3 | 2 | 3 | 3 |
| 2 | 1 | 4 | 5 | 4 | 4 | 3 |
| 3 | 1 | 3 | 3 | 3 | 3 | 2 |
| 4 | 1 | 3 | 4 | 3 | 4 | 4 |
| 5 | 2 | 4 | 3 | 3 | 3 | 3 |
| 6 | 2 | 4 | 4 | 4 | 4 | 3 |
| mean value | 1.5 | 3.7 | 3.7 | 3.2 | 3.5 | 3.0 |

TABLE 14

Isoamyl p-Methoxycinnamate
Isoamyl p-Methoxycinnamate + 1,2 heptanediol, 85 : 15 w/w % (B, B1, B2) Isoamyl p-Methoxycinnamate, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 3 | 3 | 1 | 4 | 1 |

TABLE 15

Homosalate
Homosalate + 1,2 heptanediol, 85 : 15 w/w % (B, B1, B2) Homosalate, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 4 | 2 | 4 | 4 | 4 | 3 |
| 2 | 2 | 3 | 3 | 3 | 3 | 3 |
| 3 | 1 | 2 | 2 | 1 | 2 | 1 |
| 4 | 1 | 2 | 2 | 3 | 2 | 1 |
| 5 | 1 | 3 | 2 | 4 | 2 | 4 |
| 6 | 3 | 5 | 3 | 4 | 4 | 3 |
| mean value | 2.0 | 2.8 | 2.7 | 3.2 | 2.8 | 2.5 |

TABLE 16

Chlorella oil (biotech)
Chlorella oil + 1,2 heptanediol, 85 : 15 w/w % (B, B1, B2) Chlorella oil, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 1 | 4 | 3 | 2 | 3 | 2 |
| 2 | 2 | 4 | 2 | 3 | 2 | 3 |
| 3 | 1 | 4 | 2 | 3 | 3 | 4 |
| 4 | 1 | 5 | 3 | 2 | 3 | 4 |
| 5 | 1 | 3 | 4 | 3 | 4 | 2 |
| 6 | 2 | 4 | 4 | 3 | 4 | 3 |
| mean value | 1.3 | 4.0 | 3.0 | 2.7 | 3.2 | 3.0 |

TABLE 17

Microalgae oil
Microalgae oil + 1,2 heptanediol, 85 : 15 w/w % (B, B1, B2) Microalgae oil, neat (A, A1, A2)

| | A | B | A1 | B1 | A2 | B2 |
|---|---|---|---|---|---|---|
| Panellist | spreading | | fatty/greasy skin feel | | absorption/ remaining residue | |
| 1 | 2 | 3 | 3 | 1 | 4 | 2 |
| 2 | 3 | 5 | 3 | 3 | 4 | 3 |
| 3 | 2 | 4 | 5 | 3 | 4 | 2 |
| 4 | 2 | 4 | 4 | 3 | 4 | 2 |
| 5 | 1 | 4 | 3 | 2 | 3 | 2 |
| 6 | 3 | 3 | 3 | 4 | 3 | 4 |
| mean value | 2.2 | 3.8 | 3.5 | 2.7 | 3.7 | 2.5 |

It was found that 1,2-heptanediol improves the spreading behaviour and/or significantly reduces the fatty/greasy skin feel of oily ingredients. In parallel, the absorption (less remaining residue) was improved too.

### Example 3: Sensory Properties of Topical Compositions According to the Present Invention

For evaluating the sensory behaviour of mixtures including a 1,2-alkanediol and a 2,3-alkanediol in combination with different liquid lipophilic components, oil components specified in Table 1 were blended with different alkanediol mixtures including a 1,2-alkanediol and a 2,3-alkanediol in a ratio as specified in the following tables and assessed by untrained panellists in comparison to the liquid lipophilic component without addition of the alkanediol mixture (placebo).

The test procedure was the same as described in Example 2.

### Results

TABLE 18

Caprylic Capric Triglyceride (CCT)
CCT + 1,2-hexanediol; w/w % 85 : 15 (B, B1; B2) CCT, neat (A, A1, A2)

| | A | B | A1 | B1 | A2 | B2 |
|---|---|---|---|---|---|---|
| Panelist | spreading | | fatty/greasy skin feel | | remaining residue/absorption | |
| 1 | 3 | 4 | 5 | 4 | 4 | 3 |
| 2 | 1 | 3 | 4 | 3 | 5 | 3 |
| 3 | 1 | 4 | 4 | 3 | 4 | 2 |
| 4 | 2 | 3 | 5 | 3 | 5 | 3 |
| 5 | 2 | 4 | 5 | 3 | 5 | 3 |
| mean value | 1.8 | 3.6 | 4.6 | 3.2 | 4.6 | 2.8 |
| CCT +2,3-hexanediol w/w % 85 : 15 (B, B1; B2) CCT, neat (A, A1, A2) | | | | | | |
| | A | B | A1 | B1 | A2 | B2 |
| Panelist | spreading | | fatty/greasy skin feel | | remaining residue/absorption | |
| 1 | 3 | 5 | 5 | 4 | 4 | 2 |
| 2 | 1 | 2 | 4 | 4 | 5 | 4 |
| 3 | 1 | 4 | 4 | 3 | 4 | 3 |
| 4 | 2 | 1 | 5 | 2 | 5 | 2 |
| 5 | 2 | 4 | 5 | 3 | 5 | 3 |
| mean value | 1.8 | 3.2 | 4.6 | 3.2 | 4.6 | 2.8 |
| 1 | 3 | 4 | 5 | 3 | 4 | 3 |
| 2 | 1 | 2 | 4 | 3 | 5 | 2 |
| 3 | 1 | 5 | 4 | 2 | 4 | 2 |
| 4 | 2 | 3 | 5 | 4 | 5 | 3 |
| 5 | 2 | 4 | 5 | 3 | 5 | 3 |
| mean value | 1.8 | 3.6 | 4.6 | 3.0 | 4.6 | 2.6 |

The blend of caprylic capric triglycerides (CCT) with 1,2-hexandiol and 2,3-hexanediol (ratio 95 : 5) showed less fatty/greasy skin feel and in addition less remaining residue on skin (indicates better absorption) versus 1,2-hexanediol and 2,3-hexanediol alone.

TABLE 19

Cetearyl Nonanoate (SymMollient® S)
Cetearyl Nonanoate + 1,2-heptanediol; w/w % 85 : 15 (B, B1; B2) Cetearyl Nononate, neat (A, A1, A2)

| | A | B | A1 | B1 | A2 | B2 |
|---|---|---|---|---|---|---|
| Panelist | spreading | | fatty/greasy skin feel | | remaining residue/absorption | |
| 1 | 2 | 4 | 5 | 3 | 5 | 3 |
| 2 | 2 | 4 | 5 | 4 | 5 | 3 |
| 3 | 3 | 4 | 5 | 4 | 5 | 4 |
| 4 | 2 | 4 | 4 | 3 | 4 | 3 |
| 5 | 2 | 4 | 5 | 3 | 5 | 3 |
| mean value | 2.2 | 4.0 | 4.8 | 3.4 | 4.8 | 3.2 |
| 1 | 2 | 3 | 5 | 5 | 5 | 5 |
| 2 | 2 | 4 | 5 | 4 | 5 | 5 |
| 3 | 3 | 4 | 5 | 4 | 5 | 4 |
| 4 | 2 | 3 | 4 | 3 | 4 | 3 |
| 5 | 2 | 4 | 5 | 2 | 5 | 2 |
| mean value | 2.2 | 3.6 | 4.8 | 3.6 | 4.8 | 3.8 |
| 1 | 2 | 5 | 5 | 5 | 5 | 4 |
| 2 | 2 | 5 | 5 | 4 | 5 | 4 |
| 3 | 3 | 5 | 5 | 4 | 5 | 4 |
| 4 | 2 | 4 | 4 | 3 | 4 | 3 |
| 5 | 2 | 5 | 5 | 3 | 5 | 3 |
| mean value | 2.2 | 4.8 | 4.8 | 3.8 | 4.8 | 3.6 |

The blend of Cetearyl Nonanoate with 1,2-heptanediol and 2,3-heptanediol (ratio 95:5) showed significant better spreading properties versus 1,2-heptanediol and 2,3-heptanediol alone.

TABLE 20

Octocrylene (NeoHeliopan® 303)
Octocrylene + 1,2-heptanediol; w/w % 85 : 15 (B, B1; B2) Octocrylene, neat (A, A1, A2)

| | A | B | A1 | B1 | A2 | B2 |
|---|---|---|---|---|---|---|
| Panelist | spreading | | fatty/greasy skin feel | | remaining residue/absorption | |
| 1 | 1 | 4 | 5 | 4 | 5 | 4 |

The blend of Octocrylene with 1,2-heptanediol and 2,3-heptanediol (ratio 95 : 5) showed better spreading properties, less fatty/greasy skin feel and better absorption versus the blend of octocrylene with 1,2- and 2,3-heptanediol each alone.

TABLE 21

Octocrylene (NeoHeliopan® 303)
Octocrylene + 1,2-octanediol w/w % 85 : 15 (B, B1; B2) Octocrylene, neat (A, A1, A2)

| | A | B | A1 | B1 | A2 | B2 |
|---|---|---|---|---|---|---|
| Panelist | spreading | | fatty/greasy skin feel | | remaining residue/absorption | |
| 1 | 1 | 4 | 3 | 4 | 3 | 3 |
| 2 | 1 | 3 | 5 | 3 | 5 | 4 |
| mean value | 1.0 | 3.5 | 4.0 | 3.5 | 4.0 | 3.5 |
| Octocrylene + 2,3-octanediol w/w % 85 : 15 (B, B1; B2) Octocrylene, neat (A, A1, A2) | | | | | | |
| | A | B | A1 | B1 | A2 | B2 |
| Panelist | spreading | | fatty/greasy skin feel | | remaining residue/absorption | |
| S | 1 | 3 | 3 | 2 | 3 | 2 |
| N | 1 | 4 | 5 | 4 | 5 | 3 |
| mean value | 1.0 | 3.5 | 4.0 | 3.0 | 4.0 | 2.5 |
| Octocrylene + Blend of 1,2- and 2,3-octanediol (95:5) w/w% 85:15 (B, B1; B2) Octocrylene, neat (A, A1, A2) | | | | | | |
| | A | B | A1 | B1 | A2 | B2 |
| Panelist | spreading | | fatty/greasy skin feel | | remaining residue/absorption | |
| 1 | 1 | 5 | 3 | 2 | 3 | 2 |
| 2 | 1 | 4 | 5 | 3 | 5 | 3 |
| mean value | 1.0 | 4.5 | 4.0 | 2.5 | 4.0 | 2.5 |

The blend of Octocrylene with 1,2-octanediol and 2,3-octanediol (ratio 95:5) showed better spreading properties, less fatty /greasy skin feel and better absorption versus the blend of Octocrylene with 1,2- and 2,3-octanediol each alone.

### Example 4: Sensory Properties of Topical Formulations According to the Present Invention

For evaluating the sensory behaviour, liquid lipophilic components specified in the following Table 22 were blended with 1,2-heptanediol or 2,3-heptanediol or a mixture of 1,2-heptanediol and 2,3-heptanediol or 1,2-octanediol or 2,3-octanediol or a mixture of 1,2-octanediol and 2,3-octanediol (formulation) in a ratio of 85 : 15 and assessed by untrained panellists in comparison to the liquid lipophilic component without addition of the specified alkanediols (placebo).

The test procedure was carried out in the same manner as described in Example 2.

The test results are summarized in the following Table 22.

TABLE 22

| | Liquid lipophilic component | alkanediol | Spreading | | | Fatty/greasy skin feel | | | Absorption/ remaining residue | | |
|---|---|---|---|---|---|---|---|---|---|---|---|
| | | | without | with | Delta *) | without | with | Delta *) | without | with | Delta *) |
| Plant Oil | Argan Oil | 1,2-heptanediol | 2 | 4.2 | 2.2 | 4 | 2.4 | 1.6 | 3.8 | 2.8 | 1 |
| Plant Oil | Argan Oil | 2,3-heptanediol | 2 | 3 | 1 | 4 | 2.6 | 1.4 | 3.8 | 2.4 | 1.4 |
| Plant Oil | Argan Oil | 1,2-heptanediol/ 2,3-heptanediol (95:5) | 2 | 3.8 | 1.8 | 4 | 2.6 | 1.4 | 3.8 | 2.4 | 1.4 |
| Plant Oil | Argan Oil | 1,2-octanediol | 2 | 3.8 | 1.8 | 4 | 2.6 | 1.4 | 3.8 | 2.6 | 1.2 |
| Plant Oil | Argan Oil | 2,3-octanediol | 2 | 3.4 | 1.4 | 4 | 2.4 | 1.6 | 3.8 | 2.2 | 1.6 |
| Plant Oil | Argan Oil | 1,2-octanediol/ 2,3-octanediol (95:5) | 2 | 4.2 | 2.2 | 4 | 2.2 | 1.8 | 3.8 | 2 | 1.8 |
| Plant Oil | Avocado Oil | 1,2-heptanediol | 2.2 | 4.4 | 2.2 | 3.8 | 2.6 | 1.2 | 3.8 | 2.6 | 1.2 |
| Plant Oil | Avocado Oil | 2,3-heptanediol | 2.2 | 3.8 | 1.6 | 3.8 | 2 | 1.8 | 3.8 | 2.4 | 1.4 |
| Plant Oil | Avocado Oil | 1,2-heptanediol/ 2,3-heptanediol (95:5) | 2.2 | 3.6 | 1.4 | 3.8 | 2.2 | 1.6 | 3.8 | 2 | 1.8 |
| Plant Oil | Avocado Oil | 1,2-octanediol | 2 | 4 | 2 | 3.8 | 3 | 0.8 | 3.8 | 2.6 | 1.2 |
| Plant Oil | Avocado Oil | 2,3-octanediol | 2 | 3.2 | 1.2 | 3.8 | 2.8 | 1 | 3.8 | 2.4 | 1.4 |
| Plant Oil | Avocado Oil | 1,2-octanediol/ 2,3-octanediol (95:5) | 2 | 3.8 | 1.8 | 3.8 | 2.2 | 1.6 | 3.8 | 2.2 | 1.6 |
| Plant Oil | Butyrospermum Parkii (Shea) Butter | 1,2-heptanediol | 2 | 4 | 2 | 4 | 2 | 2 | 4.2 | 2.7 | 1.5 |
| Plant Oil | Cacao butter | 1,2-heptanediol | 1.8 | 3.8 | 2 | 4.2 | 3 | 1.2 | 3.6 | 3.6 | 0 |
| Plant Oil | Cacao butter | 2,3-heptanediol | 1.8 | 3 | 1.2 | 4.2 | 3.4 | 0.8 | 3.6 | 3.2 | 0.4 |
| Plant Oil | Cacao butter | 1,2-heptanediol/ 2,3-heptanediol (95:5) | 1.8 | 4 | 2.2 | 4.2 | 3 | 1.2 | 3.6 | 3 | 0.6 |
| Plant Oil | Cacao butter | 1,2-octanediol | 1.8 | 3.8 | 2 | 4.2 | 3.2 | 1 | 3.6 | 3 | 0.6 |
| Plant Oil | Cacao butter | 2,3-octanediol | 1.8 | 3 | 1.2 | 4.2 | 3 | 1.2 | 3.6 | 2.8 | 0.8 |
| Plant Oil | Cacao butter | 1,2-octanediol/ 2,3-octanediol (95:5) | 1.8 | 2.6 | 0.8 | 4.2 | 3.2 | 1 | 3 | 2.6 | 0.4 |
| Plant Oil | Caprylic Capric Triglyceride (neutral oil) | 1,2-heptanediol | 1.9 | 3.4 | 1.5 | 3.2 | 2.9 | 0.3 | 3.3 | 2.7 | 0.6 |
| Plant Oil | Caprylic Capric Triglyceride (neutral oil) | 2,3-heptanediol | 1.9 | 3.6 | 1.7 | 3.2 | 3.4 | -0.2 | 3.3 | 3 | 0.3 |
| Plant Oil | Caprylic Capric Triglyceride (neutral oil) | 1,2-heptanediol/ 2,3-heptanediol (95:5) | 1.9 | 3.8 | 1.9 | 3.2 | 2.6 | 0.6 | 3.3 | 2.8 | 0.5 |
| Plant Oil | Castor Oil | 1,2-heptanediol | 1.2 | 2.2 | 1 | 4.2 | 2.8 | 1.4 | 4.4 | 4 | 0.4 |
| Plant Oil | Castor Oil | 2,3-heptanediol | 1.2 | 1.6 | 0.4 | 4.2 | 3.6 | 0.6 | 4.4 | 3.8 | 0.6 |
| Plant Oil | Castor Oil | 1,2-heptanediol/ 2,3-heptanediol (95:5) | 1.2 | 1.8 | 0.6 | 4.2 | 3.4 | 0.8 | 4.4 | 3.4 | 1 |
| Plant Oil | Castor Oil | 1,2-octanediol | 1.2 | 1.6 | 0.4 | 4.2 | 3.6 | 0.6 | 4.4 | 3.4 | 1 |
| Plant Oil | Castor Oil | 2,3-octanediol | 1.2 | 1.8 | 0.6 | 4.2 | 2.8 | 1.4 | 4.4 | 3 | 1.4 |
| Plant Oil | Castor Oil | 1,2-octanediol/ 2,3-octanediol (95:5) | 1.2 | 2 | 0.8 | 4.2 | 3.4 | 0.8 | 4.4 | 3.2 | 1.2 |
| Plant oil | Chlorella oil (biotech) | 1,2-heptanediol | 1.3 | 4 | 2.7 | 3 | 2.7 | 0.3 | 3.2 | 3 | 0.2 |
| Plant Oil | Coconut Oil | 1,2-heptanediol | 2.4 | 4.2 | 1.8 | 3.2 | 1.8 | 1.4 | 3.4 | 2 | 1.4 |
| Plant Oil | Coconut Oil | 2,3-heptanediol | 2.4 | 3.4 | 1 | 3.2 | 2.4 | 0.8 | 3.4 | 2.4 | 1 |
| Plant Oil | Coconut Oil | 1,2-heptanediol/ 2,3-heptanediol (95:5) | 2.4 | 4.2 | 1.8 | 3.2 | 3 | 0.2 | 3.4 | 2.4 | 1 |
| Plant Oil | Coconut Oil | 1,2-octanediol | 2.4 | 4.4 | 2 | 3.6 | 2.6 | 1 | 3.8 | 3 | 0.8 |
| Plant Oil | Coconut Oil | 2,3-octanediol | 2.4 | 4.2 | 1.8 | 3.6 | 2.2 | 1.4 | 3.8 | 2.6 | 1.2 |
| Plant Oil | Coconut Oil | 1,2-octanediol/ 2,3-octanediol (95:5) | 2.4 | 4 | 1.6 | 3.6 | 2.8 | 0.8 | 3.8 | 3 | 0.8 |
| Plant Oil | Grapeseed Oil | 1,2-heptanediol | 2 | 3.8 | 1.8 | 4.2 | 2.2 | 2 | 2.6 | 2.8 | 0.2 |
| Plant Oil | Grapeseed Oil | 2,3-heptanediol | 2 | 3.6 | 1.6 | 4.2 | 2.6 | 1.6 | 3 | 2.2 | 0.8 |
| Plant Oil | Grapeseed Oil | 1,2-heptanediol/ 2,3-heptanediol (95:5) | 2 | 3.8 | 1.8 | 4.2 | 2.2 | 2 | 2.6 | 1.6 | 1 |
| Plant Oil | Grapeseed Oil | 1,2-octanediol | 2 | 4 | 2 | 4.2 | 2.8 | 1.4 | 2.6 | 1.6 | 1 |
| Plant Oil | Grapeseed Oil | 2,3-octanediol | 2 | 2.8 | 0.8 | 4.2 | 2.8 | 1.4 | 2.6 | 2.4 | 0.2 |
| Plant Oil | Grapeseed Oil | 1,2-octanediol/ 2,3-octanediol (95:5) | 2 | 3.8 | 1.8 | 4.2 | 2.2 | 2 | 2.3 | 1.4 | 0.9 |
| Plant Oil | Helianthus Annuus (Sunflower) Seed Oil | 1,2-heptanediol | 1.2 | 4.2 | 3 | 3.8 | 2.3 | 1.5 | 3.3 | 2 | 1.3 |
| Plant Oil | Helianthus Annuus (Sunflower) Seed Oil | 2,3-heptanediol | 1.6 | 3.8 | 2.2 | 3.6 | 2.6 | 1 | 3.6 | 2.6 | 1 |
| Plant Oil | Helianthus Annuus (Sunflower) Seed Oil | 1,2-heptanediol/ 2,3-heptanediol (95:5) | 1.6 | 4.4 | 2.8 | 3.6 | 2.4 | 1.2 | 3.6 | 2 | 1.6 |
| Plant Oil | Helianthus Annuus (Sunflower) Seed Oil | 1,2-octanediol | 1.4 | 4.2 | 2.8 | 3.8 | 2 | 1.8 | 3.8 | 2 | 1.8 |
| Plant Oil | Helianthus Annuus (Sunflower) Seed Oil | 2,3-octanediol | 1.4 | 3.4 | 2 | 3.8 | 3 | 0.8 | 3.8 | 3.4 | 0.4 |
| Plant Oil | Helianthus Annuus (Sunflower) Seed Oil | 1,2-octanediol/ 2,3-octanediol (95:5) | 1.4 | 4.4 | 3 | 3.8 | 2.4 | 1.4 | 3.6 | 2.6 | 1 |
| Plant Oil | Helianthus Annuus (Sunflower) Seed Oil | 1,2-heptanediol/ 1,2-octanediol (1:1) | 1.2 | 5 | 3.8 | 3.8 | 2.8 | 1 | 3.3 | 2.2 | 1.1 |
| Plant Oil | Helianthus Annuus (Sunflower) Seed Oil | 1,2-heptanediol/ 1,2-octanediol (95:5) | 1.2 | 4.2 | 3 | 3.8 | 2.8 | 1 | 3.3 | 2.4 | 0.9 |
| Plant Oil | Olive Oil | 1,2-heptanediol | 2.2 | 4 | 1.8 | 3.8 | 2 | 1.8 | 4 | 2.4 | 1.6 |
| Plant Oil | Olive Oil | 2,3-heptanediol | 2.2 | 4 | 1.8 | 3.8 | 2.2 | 1.6 | 4 | 2 | 2 |
| Plant Oil | Olive Oil | 1,2-heptanediol/ | 2.2 | 3.8 | 1.6 | 3.8 | 1.8 | 2 | 4 | 2 | 2 |
| | | 2,3-heptanediol (95:5) | | | | | | | | | |
| Plant Oil | Olive Oil | 1,2-octanediol | 2.2 | 4.2 | 2 | 3.8 | 2.6 | 1.2 | 4 | 2.6 | 1.4 |
| Plant Oil | Olive Oil | 2,3-octanediol | 2.4 | 3.2 | 0.8 | 3.8 | 2.4 | 1.4 | 4 | 2.6 | 1.4 |
| Plant Oil | Olive Oil | 1,2-octanediol/ 2,3-octanediol (95:5) | 2.2 | 4.4 | 2.2 | 3.8 | 2.2 | 1.6 | 4 | 2.4 | 1.6 |
| Plant Oil | Prunus Amygdalus Dulcis (Sweet Almond) Oil | 1,2-heptanediol | 1.3 | 3 | 1.7 | 3.3 | 2.5 | 0.8 | 3.7 | 2.2 | 1.5 |
| Plant Oil | Simmondsia Chinensis (Jojoba) Seed Oil | 1,2-heptanediol | 1.5 | 4 | 2.5 | 3.8 | 2.8 | 1 | 3.7 | 2.5 | 1.2 |
| Natural oil | Microalgae oil | 1,2-heptanediol | 2.2 | 3.8 | 1.6 | 3.5 | 2.7 | 0.8 | 3.7 | 2.5 | 1.2 |
| Fatty alcohol | Hexyldecanol (Eutanol G) | 1,2-heptanediol | 2.5 | 3.5 | 1 | 3 | 3 | 0 | 2.5 | 2.7 | 0.2 |
| Fatty ester | Cetearyl Nonanoate (SymMollient S) | 1,2-heptanediol | 1.7 | 4.2 | 2.5 | 3.2 | 2.5 | 0.7 | 3.2 | 2 | 1.2 |
| Fatty ester | Propanediol Dicaprylate/Dic aprate (Symollient PDCC) | 1,2-heptanediol | 1.6 | 2.9 | 1.3 | 2.7 | 2.5 | 0.2 | 2.5 | 2.4 | 0.1 |
| UV filter | Ethylhexyl Methoxycinnam ate (Neo Heliopan AV) | 1,2-heptanediol | 1.5 | 3.7 | 2.2 | 3.7 | 3.2 | 0.5 | 3.5 | 3 | 0.5 |
| UV filter | Ethylhexyl Salicylate (Neo Heliopan OS) | 1,2-heptanediol | 1.8 | 4.3 | 2.5 | 3.7 | 1.8 | 1.9 | 3.5 | 2.2 | 1.3 |
| UV filter | Homosalate (Neo Heliopan HMS) | 1,2-heptanediol | 2 | 2.8 | 0.8 | 2.7 | 3.2 | 0.5 | 2.8 | 2.5 | 0.3 |
| UV filter | Isoamyl p-Methoxycinnam ate (Neo Heliopan E 1000) | 1,2-heptanediol | 1.5 | 2.7 | 1.2 | 4 | 2.7 | 1.3 | 4.2 | 2.5 | 1.7 |
| UV filter | Octocrylene (Neo Heliopan 303) | 1,2-heptanediol | 1 | 2.7 | 1.7 | 1.8 | 3.3 | 1.5 | 4 | 2.7 | 1.3 |
| Plant Oil | Caprylic Capric Triglyceride (CCT) (neutral oil) | 1,2-hexanediol | 1.8 | 3.6 | 1.8 | 4.6 | 3.2 | 1.4 | 4.6 | 2.8 | 1.8 |
| Plant Oil | Caprylic Capric Triglyceride (CCT) (neutral oil) | 2,3-hexanediol | 1.8 | 3.2 | 1.4 | 4.6 | 3.2 | 1.4 | 4.6 | 2.8 | 1.8 |
| Plant Oil | Caprylic Capric Triglyceride (CCT) (neutral oil) | 1,2-hexanediol/2,3-hexanediol (95:5) | 1.8 | 3.6 | 1.8 | 4.6 | 3.0 | 1.6 | 4.6 | 2.6 | 2.0 |
| \Fatty ester | Cetearyl Nonanoate | 1,2-heptanediol | 2.2 | 4 | 1.8 | 4.8 | 3.4 | 1.4 | 4.8 | 3.2 | 1.6 |
| Fatty ester | Cetearyl Nonanoate | 2,3-heptanediol | 2.2 | 3.6 | 1.4 | 4.8 | 3.6 | 1.2 | 4.8 | 3.8 | 1.0 |
| Fatty ester | Octocrylene (Neo Heliopan 303) | 1,2-heptanediol | 1.0 | 3.5 | 2.5 | 4.0 | 3.5 | 0.5 | 4.0 | 3.5 | 0.5 |
| Fatty ester | Octocrylene (Neo Heliopan 303) | 2,3-heptanediol | 1.0 | 3.5 | 2.5 | 4.0 | 3.0 | 1.0 | 4.0 | 3.5 | 0.5 |
| Fatty ester | Octocrylene (Neo Heliopan 303) | 1,2-heptanediol/2,3-heptanediol (95:5) | 1.0 | 4.0 | 3.0 | 4.0 | 3.0 | 1.0 | 4.0 | 2.5 | 1.5 |
| Fatty ester | Octocrylene (Neo Heliopan 303) | 1,2-octanediol | 1.0 | 3.5 | 2.5 | 4.0 | 3.5 | 0.5 | 4.0 | 3.5 | 0.5 |
| Fatty ester | Octocrylene (Neo Heliopan 303) | 2,3-octanediol | 1.0 | 3.5 | 2.5 | 4 | 3.0 | 1.0 | 4.0 | 2.5 | 1.5 |
| Fatty ester | Octocrylene (Neo Heliopan 303) | 1,2-octanediol/2,3-octanediol (95:5) | 1.0 | 4.5 | 3.5 | 4.0 | 2.5 | 1.5 | 4.0 | 2.5 | 1.5 |

*) Higher delta values indicate better spreading properties, less fatty/greasy skin feel and less remaining residue on skin/ better absorption

It was found that the use of 1,2-heptanediol or 2,3-heptanediol or a mixture of 1,2-heptanediol and 2,3-heptanediol or 1,2-octanediol or 2,3-octanediol or a mixture of 1,2-octanediol and 2,3-octanediol improves the spreading behavior and/or significantly reduces the fatty/greasy skin feel of oily ingredients. In parallel, the absorption (less remaining residue) was improved too.

In particular liquid lipophilic components which have a poor spreading by nature, which initial spreading, i.e. without the addition of an alkanediol component, was scored with < 2, show a remarkably improvement in spreading both the tested single alkanediol compound and the tested alkanediol mixture. 1,2-heptanediol and a mixture of 1,2-heptanediol and 2,3-heptanediol perform best.

### Example5: Sensory Properties of a Composition According to the Present Invention in Comparison to a Composition Comprising Sunflower Oil and Cyclopentasiloxane

The sensory behavior of a composition comprising sunflower oil plus 1,2-heptanediol in a ratio of 85 : 15 was assessed by untrained panelists in comparison to a composition comprising sunflower oil plus cyclopentasiloxane in a ratio of 85 : 15.

The test procedure was carried out in the same manner as described in Example 2.

The results of the evaluation are summarized in Table 23.

TABLE 23

Sunflower oil + 1,2-Heptanediol, 85 : 15 w/w % (A, A1, A2) Sunflower oil + Cyclopentasiloxane, 85 : 15 (B, B1, B2)

| Panellist | A | B | A1 | B1 | A1 | B1 |
|---|---|---|---|---|---|---|
| Panellist | Spreading | | Fatty/greasy skin feel | | Absorption/ remaining residue | |
| 1 | 2 | 3 | 3 | 4 | 3 | 3 |
| 2 | 5 | 4 | 3 | 3 | 2 | 3 |
| 3 | 3 | 4 | 3 | 4 | 3 | 4 |
| 4 | 4 | 4 | 3 | 3 | 3 | 2 |
| 5 | 4 | 4 | 3 | 4 | 3 | 3 |
| mean value | 3.6 | 3.8 | 3.0 | 3.6 | 2.8 | 3.0 |

As it is obvious from the results from Table 23, the tested sunflower oil composition according to the present invention has a similar spreading behavior and/or reduces the fatty/greasy skin feel of oily ingredients like a sunflower oil composition comprising the same amount silicon. The tested sunflower oil composition has also a similar absorption (less remaining residue), and, thus, in overall impart a silky, spreadable, and luxurious texture. Hence, the alkanediols as defined before, in particular 1,2-heptanediol, are suitable to partially or completely replace a silicone in a cosmetic or pharmaceutical composition, in order to achieve a superior skin sensation.

### Example6: Solubility of Alkanediols in Water

For the determination of the maximum water solubility of an alkanediol, water and different concentrations of the respective alkanediol were blended by stirring. The assessment of solubility was carried out at ambient temperature after 24 h storage at 5° C. The determination of whether the alkanediol has dissolved in water is based entirely on visual observation. An alkanediol compound has dissolved if the mixture is clear and shows no signs of cloudiness or precipitation.

TABLE 24

| | | |
|---|---|---|
| Tradename/ Chemical name | INCI | Maximum solubility in water (% by weight) |
| Hydrolite® 5 green | Pentylene Glycol | 50 |
| Hydrolite® 6 | Hexanediol | 50 |
| 1,2-heptanediol | Heptanediol | 2.5 |
| Hydrolite® 8 | Caprylyl Glycol | 0.5 |
| SymDiol® 68 | Hexanediol, Caprylyl Glycol | 1.0 |
| 1,2-nonanediol | Nonanediol | 0.1 |
| SymClariol® | Decylene Glycol | - |
| 1,2-undecanediol | Undecanediol | - |
| 2,3-heptanediol | - | 3.6 |
| 2,3-octanediol | - | 1.5 |
| 2,3-nonanediol | - | 0.3 |
| 2,3-undecanediol | - | - |

### Example 7: Formulation Examples

In the following formulation examples the following five perfume oils PFO1, PFO2, PFO3, PFO4 or PFO5 were each used as fragrance.

TABLE 25

Composition of perfume oil 1 (PO1; amounts in ‰ by weight)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| Benzylacetate | 50 |
| BERGAMOT IDENTOIL® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2.4 10% IPM | 2 |
| FLOROSA | 5 |
| FRAMBINON® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 26

Composition of perfume oil 2 (PO2; amounts in ‰ by weight)

| Ingredients | Amount |
|---|---|
| Acetophenone. 10% in DPG | 10 |
| n-Undecanal | 5 |
| Aldehyde C14 (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3. 10% in DPG | 5 |
| Hexenyl salicylate cis-3 | 20 |
| Indole. 10% in DPG | 10 |
| Alpha-ionone | 15 |
| Beta-ionone | 5 |
| Lilial® (2-methyl-3-(4-tert-butylphenyl)propanal) | 60 |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 27

Composition of perfume oil 3 (PO3; amounts in ‰ by weight)

| Ingredients | Amount |
|---|---|
| Benzyl acetate | 60 |
| Citronellyl acetate | 60 |
| Cyclamenaldehyde (2-methyl-3-(4-isopropylphenyl)propanal | 20 |
| Dipropylene glycol (DPG) | 60 |
| Ethyllinalool | 40 |
| Florol (2-isobutyl-4-methyltetrahydro-2H-pyran-4-ol) | 30 |
| Globanone® [(E/Z)-8-cyclohexadecen-1-one] | 180 |
| Hedione® (methyldihydrojasmonate) | 140 |
| Hexenyl salicylate. cis-3 | 10 |
| Vertocitral (2.4-dimethyl-3-cyclohexenecarboxaldehyde) | 5 |
| Hydratropaldehyde. 10% in DPG | 5 |
| Isodamascone (1-(2.4.4-trimethyl-2-cyclohexen-1-yl)-2-buten-1-one. 10% in DPG | 5 |
| Isomuscone (cyclohexadecanone) | 40 |
| Jacinthaflor (2-methyl-4-phenyl-1.3-dioxolane) | 10 |
| Cis-jasmone. 10% in DPG | 20 |
| Linalool | 50 |
| Linalyl acetate | 30 |
| Methyl benzoate. 10% in DPG | 25 |
| para-Methyl cresol. 10% in DPG | 10 |
| Nerol | 20 |
| Phenylpropylaldehyde | 5 |
| 2-Phenylethyl alcohol | 82 |
| Tetrahydrogeraniol | 13 |
| 2.2-Dimethyl-3-cyclohexyl-1-propanol | 80 |
| Total: | 1000 |

TABLE 28

Composition of perfume oil 4 (PO4; amounts in ‰ by weight)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE® 1951 10% in DPG | 15 |
| COUMARIN | 5 |
| CYCLOGALBANATE® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE® HC/30 | 35 |
| HEDIONE® | 210 |
| HELIONAL® | 15 |
| HELVETOLIDE® (ALICYC) | 30 |
| HEXENYLSALICYLATE CIS-3 | 20 |
| ISO E SUPER® | 40 |
| LEAFOVERT® 10% in DEP | 10 |
| LILIAL® | 80 |
| LYRAL® | 20 |
| MANDARIN OIL | 10 |
| STYRALYL ACETATE | 5 |
| SYMROSE® | 15 |
| VANILLIN 10% in DEP | 20 |
| DIPROPYLENE GLYCOL (DPG) | 50 |
| TOTAL | 1000 |

TABLE 29

Composition of perfume oil 5 (PO5; amounts in ‰ by weight)

| Ingredients | Amount |
|---|---|
| AMAROCITE® | 10 |
| AMBROCENIDE® 10% in DPG | 5 |
| AMBROXIDE | 15 |
| AURELIONE® (⅞-Cyclohexadecenone) (MACRO) | 70 |
| BERGAMOT OIL. bergapten-free | 90 |
| CALONE® 1951 10% in DPG | 20 |
| CARAWAY OIL | 10 |
| CITRAL | 20 |
| COUMARIN | 10 |
| ALPHA-DAMASCONE 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL® | 5 |
| FRUCTATE® | 5 |
| GERANIUM OIL | 5 |
| HEDIONE® HC/30 | 100 |
| HELIONAL® | 10 |
| INDOLE 10% in DPG | 5 |
| ISO E SUPER® | 100 |
| KEPHALIS® | 5 |
| LAVENDER OIL | 40 |
| CITRUS OIL | 80 |
| LILIAL® | 30 |
| MANDARIN OIL | 20 |
| MUSCENONE (MACRO) | 5 |
| SANDRANOL® | 10 |
| VANILLIN 10% in DPG | 5 |
| DIPROPYLENE GLYCOL | 30 |
| TOTAL | 1000 |

The above perfume oils PO1, PO2, PO3, PO4 or PO5 were incorporated into the formulations presented below.

Cosmetic and pharmaceutical formulations (compositions); amounts are indicated as % by weight for all formulations.

TABLE30

Cream O/W

| Ingredients | INCI | Amount |
|---|---|---|
| Dracorin® CE | Glyceryl Stearate Citrate | 1.0 |
| Lanette® O | Cetearyl Alcohol | 2.0 |
| Cutina® GMS-V | Glyceryl Stearate | 1.0 |
| Tegosoft® MM | Myristyl Myristate | 1.0 |
| Hydrolite-6 | Hexanediol | 1.0 |
| Xiameter® PMX-0246. Cyclosiloxane | Cyclohexasiloxane (and) Cyclopentasiloxane | 0.5 |
| Dragoxat® 89 | Ethylhexyl Isononanoate | 2.0 |
| PCL-Liquid 100 | Cetearyl Ethylhexanoate | 4.0 |
| Neutral Oil | Caprylic/Capric Triglyceride | 4.0 |
| Carbopol® Ultrez 21 | Acrylates/C10-30 Alkyl Acrylate Crosspolymer | 0.2 |
| Keltrol® CG-T | Xanthan Gum | 0.1 |
| Water | Water (Aqua) | ad 100 |
| Glycerol 99.5 P. | Glycerol | 3.0 |
| 1.2-Propylene Glycol 99 P GC | Propylene Glycol | 2.0 |
| Sodium Benzoate | Sodium Benzoate | 0.1 |
| Sodium Hydroxide 10% solution | Sodium Hydroxide | 0.5 |
| Perfume oil PO1, PO2, PO3, PO4, or PO5 | Perfume | 0.3 |
| Euxyl® K702 | Dehydroacetic Acid, Benzoic Acid, Phenoxyethanol, Polyaminopropyl Biguanide, Ethylhexylglycerin | 0.3 |
| 1,2-heptanediol | 1.2-heptanediol | 1.0 |
| 1,2-octanediol/2,3-octanediol (95:5 w/w %) | 1,2-octanediol/2,3-octanediol (95:5 w/w %) | 1.0 |

TABLE31

Hand and body cream

| Ingredients | INCI | Amount |
|---|---|---|
| Dracorin® GOC | Glyceryl Oleate Citrate, Caprylic/Capric Triglyceride | 2.0 |
| PCL-Solid | Stearyl Heptanoate, Stearyl Caprylate | 2.5 |
| Lanette® O | Cetearyl Alcohol | 1.5 |
| Cutina® GMS-V | Glyceryl Stearate | 1.0 |
| Dragoxat® 89 | Ethylhexyl Isononanoate | 3.0 |
| PCL-Liquid 100 | Cetearyl Ethylhexanoate | 7.0 |
| Isodragol® | Triisononanoin | 4.0 |
| Xiameter® PMX-0345 Cyclosiloxane | Cyclopentasiloxane (and) Cyclohexasiloxane | 0.5 |
| Water | Water (Aqua) | ad 100 |
| Carbopol® Ultrez 21 | Acrylates/C10-30 Alkyl Acrylate Crosspolymer | 0.2 |
| Keltrol® CG-RD | Xanthan Gum | 0.1 |
| Glycerol 85 P. | Glycerol | 3.0 |
| DragoBetaGlucan | Water (Aqua). Butylene Glycol. Glycerol. Avena Sativa (Oat) Kernel Extract | 1.5 |
| Potassium Sorbate | Potassium Sorbate | 0.1 |
| Sodium Hydroxide 10% solution | Sodium Hydroxide | 0.5 |
| Perfume oil PO1, PO2, PO3, PO4, or PO5 | Fragrance | 0.2 |
| Euxyl® K300 | Methyl-, Butyl-, Ethyl-, Propyl-, Isobutylparaben, Phenoxyethan ol | 0.3 |
| 1,2-heptanediol | 1,2-heptanediol | 1.0 |
| 2,3-heptanediol | 2,3-heptanediol | 1.0 |

TABLE32

Daily face cream; SPF 20

| | |
|---|---|
| Ingredients | Amount |
| SymOcide PH Phenoxyethanol, Hydroxyacetophenone, Caprylyl Glycol, Water (Aqua) | 1 |
| Ascorbyl Palmitate Ascorbyl Palmitate | 0.1 |
| Biotive L-Arginine Arginine | 0.2 |
| Buriti oil Mauritia Flexuosa Fruit Oil | 1 |
| Cocoa butter Theobroma Cacao (Cocoa) Seed Butter | 2 |
| Dimethicone Dimethicone | 0.5 |
| Disodium EDTA Disodium EDTA | 0.1 |
| Dragosantol 100 Bisabolol | 0.1 |
| Dragoxat 89 Ethylhexyl Isononanoate | 5 |
| Emulsiphos Potassium Cetyl Phosphate, Hydrogenated Palm Glycerides | 2 |
| Extrapone Corail Glycerin, Aqua, Hydrolyzed Corallina Officinalis | 1 |
| Glycerin Glycerin | 3 |
| Isoadipate Diisopropyl Adipate | 5 |
| Jojoba Wax Flakes Hydrogenated Jojoba Oil | 1 |
| Keltrol CG-T Xanthan Gum | 0.1 |
| Lanette O Cetearyl Alcohol | 5 |
| Lanette 16 | 1 |
| Cetyl Alcohol | |
| Lanette 22 Behenyl Alcohol | 1 |
| Neo Heliopan 357 Butyl Methoxydibenzoylmethane | 3 |
| Neo Heliopan HMS Homosalate | 10 |
| Neo Heliopan Hydro used as a 25% aq. Solution neutralized by arginine Phenylbenzimidazole Sulfonic Acid | 8 |
| Neo Heliopan OS Ethylhexyl Salicylate | 5 |
| Orgasol Caresse Polyamide-5 | 1 |
| Perfume oil PO1, PO2, PO3, PO4, or PO5 | 0.1 |
| Shea butter Butyrospermum Parkii (Shea) Butter | 3 |
| Simugel EG Sodium Acrylate / Sodium Acryloyldimethyl Taurate Copolymer, Isohexadecane, Polysorbate 80 | 1 |
| SymFinity 1298 Echinacea Purpurea Extract | 0.1 |
| SymMatrix Maltodextrin. Rubus Fructicosus (Blackberry) Leaf Extract | 0.1 |
| SymSitive 1609 Pentylene Glycol, 4-t-Butylcyclohexanol | 1 |
| Tegosoft TN C12-15 Alkyl Benzoate | 4 |
| Hydrolite-8 (Caprylyl Glycol) | 0.5 |
| 1,2-heptanediol/2,3-heptanediol blend (95:5 w/w %) | 1.0 |
| Water Aqua | ad 100 |

TABLE33

Night cream W/O

| | | |
|---|---|---|
| Ingredients | INCI | Amount |
| 1,2-heptanediol | 1,2-heptanediol | 3.0 |
| 2,3-heptanediol | 2,3-heptanediol | 0.1 |
| 2,3-octanediol | 2,3-octanediol | 0.5 |
| Aloe Vera Gel Concentrate 10/1 * | Water (Aqua), Aloe Barbadensis Leaf Juice | 3.0 |
| Alugel 34 TH | Aluminium Stearate | 1.0 |
| Dragosan W/O P* | Sorbitan Isostearate, Hydrogenated Castor Oil, Ceresin, Beeswax (Cera Alba) | 6.0 |
| Dragosantol® 100* | Bisabolol | 0.2 |
| Extrapone® Witch Hazel Distillate colourless | Propylene Glycol, Hamamelis Virginiana (Witch Hazel) Water, Water (Aqua), Hamamelis Virginiana (Witch Hazel) Extract | 1.0 |
| Perfume oil PO1, PO2, PO3, PO4, or PO5 | Fragrance | 0.4 |
| Glycerol 85 % | Glycerin | 2.0 |
| Karion F | Sorbitol | 2.0 |
| Magnesium Chloride | Magnesium Chloride | 0.7 |
| Isoadipate | Diisoadipate | 2.0 |
| PCL Liquid 100 | Cetearyl Ethylhexoate | 12.0 |
| Retinyl Palmitate in Oil | Retinyl Palmitate | 0.05 |
| Hydroxypinacolone Retinoate | Hydroxypinacolone Retinoate | 0.05 |
| Sun Flower Oil | Helianthus Annuus (Sunflower) Seed Oil | 5.0 |
| Sweet Almond Oil | Prunus dulcis | 5.0 |
| SymMatrix® | Maltodextrin. Rubus Fruticosus (Blackberry) Leaf Extract | 1.0 |
| SymOcide PS | Phenoxyethanol. Decylene glycol. 1.2-Hexanediol | 1.0 |
| SymVital® AgeRepair | Zingiber Officinale (Ginger) Root Extract | 0.1 |
| Vitamin E | Tocopherol | 3.0 |
| Water (demineralized) | Water (Aqua) | ad 100 |

TABLE34

Body lotion

| Ingredients | Amount |
|---|---|
| Cetearyl Alcohol | 2.0 |
| Ethylhexyl Isononanoate | 5.0 |
| Cetearyl Ethylhexanoate. Isopropyl Myristate | 3.0 |
| Glyceryl Oleate Citrate. Caprylic/Capric Triglyceride | 4.0 |
| Water (Aqua) | ad 100 |
| Carbomer | 0.3 |
| Sodium Benzoate | 0.1 |
| Propylene Glycol | 5.0 |
| Sodium Hydroxide 30% aqueous solution | 0.3 |
| Perfume oil PO1, PO2, PO3, PO4, or PO5 | 0.3 |
| Triethylene Glycol. Imidazolidinyl Urea. Methylparaben. Propylparaben. Dehydroacetic Acid | 0.3 |
| 1,2-heptanediol | 0.2 |
| 2,3-octanediol | 0.2 |

TABLE 35

Antibacterial body lotion, sprayable

| | | |
|---|---|---|
| Ingredients | INCI | Amount |
| 1,2-heptanediol/2,3-heptanediol blend (95:5 w/w %) | 1,2-heptanediol/2,3-heptanediol blend (95:5 w/w %) | 1.5 |
| Potassium Sorbate | Potassium Sorbate | 0.2 |
| Dow Corning 345 Fluid | Cyclomethicone | 0.5 |
| Dracorin® GOC | Glyceryl Oleate Citrate, Caprylic/Capric Triglyceride | 2.0 |
| Drago-Calm | Water, Glycerin, Avena Sativa (Oat) Kernel Extract | 1.0 |
| Dragosantol® 100* | Bisabolol | 0.1 |
| Perfume oil PO1, PO2, PO3, PO4, or PO5 | Fragrance | 0.3 |
| Hydrolite®-5 | Pentylene Glycol | 5.0 |
| Neutral Oil | Caprylic/Capric Triglyceride | 4.0 |
| Paraffin Oil | Mineral Oil | 4.0 |
| PCL Liquid 100 | Cetearyl Ethylhexoate | 7.0 |
| Pemulen TR-2 | Acrylates/C10-30 Alkyl Acrylate Crosspolymer | 0.2 |
| Sodium Hydroxide (10% sol.) | Sodium Hydroxide | 0.4 |
| SymDeo® MPP | Dimethyl Phenylbutanol | 0.5 |
| SymRelief® 100 | Bisabolol, Zingiber Officinale (Ginger) Root Extract | 0.1 |
| Water (demineralized) | Water (Aqua) | ad 100 |

TABLE 36

Aseptic wound cream

| Ingredients | Amount |
|---|---|
| Sorbitan Isostearate. Hydrogenated Castor Oil. Ceresin. Beeswax (Cera Alba) | 6.0 |
| Petrolatum | 21.0 |
| Cera Alba | 5.0 |
| Cetearyl Alcohol | 7.0 |
| Cocos Nucifera (Coconut) Oil | 7.0 |
| Lanolin | 5.0 |
| Paraffinum Liquidum | 12.0 |
| Perfume oil PO1, PO2, PO3, PO4, or PO5 | 0.3 |
| Water (Aqua) | ad 100 |
| Panthenol | 7.0 |
| Magnesium Sulfate | 0.7 |
| Pentylene Glycol | 1.0 |
| Tocopheryl Acetate | 1.0 |
| Octenidine dihydrochloride | 0.1 |
| Phenoxyethanol | 0.5 |
| 1.2-heptanediol | 0.4 |
| 2.3-heptanediol | 0.01 |

TABLE 37

Anti Acne balm

| | | |
|---|---|---|
| Ingredients | INCI | Amount |
| 1,2-heptanediol / 1,2-octanediol blend (1:1 w/w %) | 1,2-heptanediol / 1,2-octanediol blend (1:1 w/w %) | 3.0 |
| SymClariol | Decylene Glycol | 0.2 |
| Abil 350 | Dimethicone | 1.0 |
| Allantoin | Allantoin | 0.1 |
| Aloe Vera Gel Concentrate 10/1 * | Water (Aqua), Aloe Barbadensis Leaf Juice | 3.0 |
| Azelaic Acid | Azelaic Acid | 5.0 |
| Cetiol OE | Dicaprylyl Ether | 4.0 |
| Cetiol SB 45 | Butyrospermum Parkii (Shea Butter) | 1.0 |
| D-Panthenol | Panthenol | 1.0 |
| Emulsiphos® | Potassium Cetyl Phosphate. Hydrogenated Palm Glycerides | 2.0 |
| Perfume oil PO1, PO2, PO3, PO4, or PO5 | Fragrance | 0.2 |
| Frescolat®ML cryst. | Menthyl Lactate | 0.8 |
| Glycerol 85% | Glycerin | 4.0 |
| Hydroviton® PLUS | Water, Pentylene Glycol, Glycerin, Fructose, Urea, Citric Acid, Sodium Hydroxide, Maltose, Sodium PCA, Sodium Chloride, Sodium Lactate, Trehalose, Allantoin, Sodium hyaluronate, Glucose | 1.0 |
| Lara Care A-200 | Galactoarabinan | 0.3 |
| Pemulen TR-2 | Acrylates/C10-30 Alkyl Acrylate Crosspolymer | 0.2 |
| Sodium Hydroxide (10% sol.) | Sodium Hydroxide | 0.4 |
| SymOcide PH | Hydroxyacetophenone, Phenoxyethanol, Caprylyl glycol, Aqua | 1.0 |
| Tegosoft TN | C12-15 Alkyl Benzoate | 5.0 |
| Tocopherol Acetate | Tocopheryl Acetate | 0.5 |
| Water (demineralized) | Water (Aqua) | ad 100 |

TABLE 38

Barrier repair cream

| | | |
|---|---|---|
| Ingredients | INCI | Amount |
| 2,3-octanediol | 2,3-octanediol | 0.5 |
| 1,2-heptanediol/2,3-heptanediol blend (95:5 w/w %) | 1,2-heptanediol/2,3-heptanediol blend (95:5 w/w %) | 0.5 |
| Abil 350 | Dimethicone | 0.5 |
| Allantoin | Allantoin | 0.25 |
| Ceramide BIO* | Cetylhydroxyproline Palmitamide | 0.5 |
| Dracorin® CE | Glyceryl Stearate Citrate | 1.5 |
| Dragoxat® 89 | Ethylhexylisononanoate | 2.0 |
| Emulsiphos® | Potassium Cetyl Phosphate, Hydrogenated Palm Glycerides | 2.0 |
| Extrapone® Rosemary GW | Glycerin, Water (Aqua), Rosmarinus officinalis (Rosemary) Leaf Extract | 0.5 |
| Perfume oil PO1, PO2, PO3, PO4, or PO5 | Fragrance | 0.1 |
| Glycerol 85 % | Glycerin | 3.0 |
| Glyceryl Stearate | Glyceryl Stearate | 2.0 |
| Hydroviton® 24 | Water, Glycerin, Sodium Lactate, TEA Lactate, Serine, Lactic Acid, Urea, Sorbitol, Sodium Chloride, Lauryl Diethylenediaminoglycine, Lauryl Aminopropylglycine. Allantoin | 1.0 |
| Isodragol® | Triisononanoin | 3.0 |
| Lanette O | Cetearyl Alcohol | 2.0 |
| NaOH 10% sol. | Sodium Hydroxide | 0.3 |
| Neutral Oil | Caprylic/Capric Triglyceride | 10.0 |
| SymCalmin® | Pentylene Glycol, Butylene Glycol, Hydroxyphenyl Propamidobenzoic Acid | 1.0 |
| SymRepair® 100 | Hexyldecanol, Bisabolol, Cetylhydroxyproline Palmitamide, Stearic Acid, Brassica Campestris (Rapeseed) Sterols | 2.0 |
| SymTriol | Caprylyl glycol, 1,2-Hexanediol, Methylbenzyl alcohol | 1.0 |
| Tegosoft PC 31 | Polyglyceryl 3-Caprate | 0.3 |
| Tocopherol Acetate | Tocopheryl Acetate | 0.3 |
| Water (demineralized) | Water (Aqua) | ad 100 |

TABLE 39

Skin soothing lotion

| | | |
|---|---|---|
| Ingredients | INCI | Amount |
| 2,3-heptanediol | 2,3-heptanediol | 0.5 |
| 2,3-octanediol | 2,3-octanediol | 0.5 |
| Abil 350 | Dimethicone | 2.0 |
| Allantoin | Allantoin | 0.2 |
| Carbopol Ultrez-10 | Carbomer | 0.1 |
| Ceramide BIO* | Cetylhydroxyproline Palmitamide | 0.1 |
| Citric Acid 10% sol. | Citric Acid | 0.4 |
| Emulsiphos® | Potassium Cetyl Phosphate, Hydrogenated Palm Glycerides | 2.0 |
| Extrapone® Green Tea GW | Glycerin, Water (Aqua), Camellia Sinensis Leaf Extract | 0.2 |
| Extrapone® Rosemary GW | Glycerin, Water (Aqua), Rosmarinus officinalis (Rosemary) Leaf Extract | 0.3 |
| Perfume oil PO1, PO2, PO3, PO4, or PO5 | Fragrance | 0.3 |
| Glycerol 85% | Glycerin | 2.0 |
| Glyceryl Stearate | Glyceryl Stearate | 2.0 |
| Isodragol® | Triisononanoin | 2.0 |
| Keltrol RD | Xanthan Gum | 0.1 |
| Lanette O | Cetearyl Alcohol | 3.0 |
| Neo PCL wssl. N | Trideceth-9. PEG-5 Ethylhexanoate. Water | 1.0 |
| PCL Liquid 100 | Cetearyl Ethylhexanoate | 5.0 |
| PCL Solid | Stearyl Heptanoate. Stearyl Caprylate | 2.0 |
| Propylene Glycol | Propylene Glycol | 5.0 |
| Sodium Hydroxide (10% sol.) | Sodium Hydroxide | 0.3 |
| SymCalmin® | Pentylene Glycol, Butylene Glycol, Hydroxyphenyl Propamidobenzoic Acid | 2.0 |
| SymMatrix® | Maltodextrin, Rubus Fruticosus (Blackberry) Leaf Extract | 0.1 |
| SymSave H | Hydroxyacetophenone | 0.4 |
| 2-Phenoxyethyl Alcohol | Phenoxyethanol | 0.4 |
| SymSitive®1609 | Pentylene Glycol, 4-t-Butylcyclohexanol | 1.5 |
| Water (demineralized) | Water (Aqua) | ad 100 |

TABLE40

Baby nappy rash cream W/O

| Ingredients | Amount |
|---|---|
| SymOcide PH Phenoxyethanol, Hydroxyacetophenone, Caprylyl Glycol, Water (Aqua) | 1 |
| Cupuaçu butter Theobroma Grandiflorum Seed Butter | 1 |
| Cutina HR Powder Hydrogenated Castor Oil | 1.5 |
| Dehymuls PGPH Polyglyceryl-2 Dipolyhydroxystearate | 5 |
| Glycerin | 5 |
| Glycerin | |
| Jojoba oil Simmondsia Chinensis (Jojoba) Seed Oil | 5 |
| Magnesium Sulfate Hepta Hydrate Magnesium Sulfate | 0.5 |
| Monomuls 90-018 Glyceryl Oleate | 1 |
| Neutral oil Caprylic/capric triglyceride | 8 |
| PCL Liquid 100 Cetearyl Ethylhexanoate | 5 |
| SymCalmin Butylene Glycol. Pentylene Glycol. Hydroxyphenyl Propamidobenzoic Acid | 1 |
| Tamanu oil Calophyllum Inophyllum Seed Oil | 0.2 |
| Tetrasodium EDTA Tetrasodium EDTA | 0.1 |
| Titan dioxide Titan dioxide | 4 |
| Water Aqua | ad 100 |
| Wheat germ oil Triticum Vulgare (Wheat) Germ Oil | 2 |
| Zinc oxide Zinc oxide | 10 |
| 1,2-heptanediol | 1.0 |

TABLE41

Skin lightening day cream O/W

| | | |
|---|---|---|
| Ingredients | INCI | Amount |
| 1,2-heptanediol/2,3-heptanediol blend (95:5 w/w %) | 1,2-heptanediol/2,3-heptanediol blend (95:5 w/w %) | 3.0 |
| Abil 350 | Dimethicone | 0.5 |
| Dracorin® CE | Glyceryl Stearate Citrate | 2.5 |
| Dracorin® GOC | Glyceryl Oleate Citrate, Caprylic/Capric Triglyceride | 0.5 |
| Drago-Beta-Glucan | Water (Aqua), Butylene Glycol, Glycerin, Avena Sativa (Oat), Kernel Extract | 0.3 |
| Dragosantol® 100* | Bisabolol | 0.2 |
| Perfume oil PO1, PO2, PO3, PO4, or PO5 | Fragrance | 0.1 |
| Frescolat®MGA | Menthone Glycerol Acetal | 0.5 |
| Glycerol 85% | Glycerin | 3.0 |
| Isopropyl Palmitate | Isopropyl Palmitate | 4.0 |
| Keltrol RD | Xanthan Gum | 0.2 |
| Lanette 16 | Cetyl Alcohol | 1.0 |
| Neo Heliopan® AV | Ethylhexyl Methoxycinnamate | 5.0 |
| Neutral Oil | Caprylic/Capric Triglyceride | 6.0 |
| PCL Liquid 100 | Cetearyl Ethylhexoate | 3.0 |
| Sodium Benzoate | Sodium Benzoate | 0.1 |
| Symdiol®68T | 1,2-Hexanediol, Caprylylglycol, Tropolone | 0.5 |
| SymVital® AgeRepair | Zingiber Officinale (Ginger) Root Extract | 0.1 |
| SymWhite®377 | Phenylethyl Resorcinol | 0.5 |
| Water (demineralized) | Water (Aqua) | ad 100 |

TABLE42

After shave tonic

| Ingredients | INCI | Amount |
|---|---|---|
| SymSol® PF-3 | Water (Aqua), Pentylene Glycol, Sodium Lauryl Sulfoacetate, Sodium Oleoyl Sarcosinate, Sodium Chloride, Disodium Sulfoacetate, SodiumOleate. Sodium Sulfate | 3.0 |
| SymSitive® 1609 | Pentylene Glycol, 4-t-Butylcyclohexanol | 1.0 |
| Frescolat® ML | Menthyl Lactate | 0.3 |
| Glycerol 99.5 P. | Glycerol | 5.0 |
| Water | Water (Aqua) | ad 100 |
| Extrapone® Glacier Water GW | Glycerol, Water (Aqua) | 1.0 |
| SymCalmin® | Butylene Glycol, Pentylene Glycol, Hydroxyphenyl Propamidobenzoic Acid | 0.5 |
| Dragosine® | Carnosine | 0.1 |
| Hydrolite® 5 | Pentylene Glycol | 5.0 |
| Ethanol 96% | Alcohol Denat. | 5.0 |
| Colour Pigment | Colour Pigment | 0.05 |
| Perfume oil PO1, PO2, PO3, PO4, or PO5 | Fragrance | 0.15 |
| 1,2 -octanediol/2,3-octanediol blend (99:1) | 1,2-octanediol/2,3-octanediol blend (99:1) | 0.5 |

TABLE 43

Antidandruff hair conditioner

| Ingredients | INCI | Amount |
|---|---|---|
| Lanette® O | Cetearyl Alcohol | 4.0 |
| Dragoxat 89 | Ethylhexyl Isononanoate | 2.0 |
| Genamin® KDM-P | Behentrimonium Chloride | 1.0 |
| SF 1550 | Phenyl Trimethicone | 0.1 |
| Neo Heliopan® BB | Benzophenone-3 | 0.1 |
| Crinipan® AD | Climbazole | 0.4 |
| Crinipan® PMC Green | Propanediol Caprylate | 1.0 |
| Glycerol 99.5 P. | Glycerol | 6.0 |
| Water | Water (Aqua) | ad 100 |
| Extrapone® Bamboo P | Propylene Glycol, Water (Aqua), Butylene Glycol, Bambusa Vulgaris Shoot Extract | 0.5 |
| Sodium Hydroxide 10% solution | Sodium Hydroxide | 0.4 |
| Colour I | Colour | 0.6 |
| Colour II | Colour | 0.3 |
| Perfume oil PO1, PO2, PO3, PO4, or PO5 | Fragrance | 0.4 |
| Preservative | Methylparaben | 0.3 |
| 2,3-heptanediol | 2,3-heptanediol | 0.3 |
| 2,3-octanediol | 2,3-octanediol | 0.3 |

TABLE 44

Hair conditioner with UV protection

| | | |
|---|---|---|
| Ingredients | INCI | Amount |
| Renex PEG 6000 | PEG-150 | 2.5 |
| Hair Conditioner Base | Cetyl alcohol, Behentrimonium chloride, Triticum Vulgare (Wheat) bran extract, linoleic acid | 3.0 |
| PCL-Solid | Stearyl heptanoate, stearyl caprylate | 0.5 |
| Dow Corning 5200 | Laurylmethicone copolyol | 0.5 |
| Natrosol 250 HR | Hydroxyethylcellulose | 0.5 |
| Benzophenone-4 | Benzophenone-4 | 1.0 |
| Neo Heliopan AP | Disodiumphenyldibenz-imidazole tetrasulphonate | 1.0 |
| Amino methyl propanol | Amino methyl propanol | 2.0 |
| Dow Corning 949 cationic emulsion | Amodimethicone, cetrimonium chloride, trideceth-12 | 2.0 |
| Perfume oil PO1, PO2, PO3, PO4, or PO5 | Fragrance | 0.8 |
| Hydrolite-6 | 1,2-Hexanediol | 0.5 |
| 1,2-heptanediol | 1,2-heptanediol | 0.5 |
| 2,3-octanediol | 2,3-octanediol | 0.01 |
| Water | Water (Aqua) | ad 100 |

TABLE 45

Hair conditioner, leave on

| | | |
|---|---|---|
| Ingredients | INCI | Amount |
| 1,2-heptanediol/2,3-heptanediol blend (95:5 w/w %) | 1,2-heptanediol/2,3-heptanediol blend (95:5 w/w %) | 1.0 |
| Dehyquart A CA | Cetrimonium Chloride | 0.2 |
| Dehyquart SP | Quaternium-52 | 2.0 |
| Dracorin® CE | Glyceryl Stearate Citrate | 1.0 |
| Drago-Calm | Water, Glycerin, Avena Sativa (Oat) Kernel Extract | 2.0 |
| Farnesol | Farnesol | |
| Perfume oil PO1, PO2, PO3, PO4, or PO5 | Fragrance | 0.5 |
| Lara Care A-200 | Galactoarabinan | 0.1 |
| Polymer JR 400 | Polyquaternium-10 | 0.1 |
| Propylene Glycol | Propylene Glycol | 0.8 |
| SymMollient® WS | Trideceth-9, PEG-5 Isononanoate, Water | 1.0 |
| SymSol®PF3 * | Water, Pentylene Glycol, Sodium Lauryl Sulfoacetate, Sodium Oleoyl Sarcosinate, Sodium Chloride, Disodium Sulfoacetate, Sodium Oleate, Sodium Sulfate | 1.5 |
| SymTriol® | Caprylyl Glycol, 1,2-Hexanediol, Methylbenzyl Alcohol | 1.0 |
| Water (demineralized) | Water (Aqua) | ad 100 |

TABLE 46

Deodorant stick

| Ingredients | Amount |
|---|---|
| Sodium stearate | 8.0 |
| PPG-3 Myristyl ether | 70.0 |
| 1,2-propylene glycol | 10.0 |
| 1,1-dimethyl-3-phenylpropanol | 0.2 |
| 2-butyloctanoic acid | 0.2 |
| Perfume oil PO1, PO2, PO3, PO4, or PO5 | 0.6 |
| Water | ad 100 |
| SymDeo Plus (Jasmol (2-benzlheptanol), 1-Dodecanol (Lauryl Alcohol), 1.2-Decanediol (Decylene Glycol), 2-Phenoxyethyl Alcohol (Phenoxyethanol)) | 0.5 |
| 2,3-heptanediol | 0.1 |
| 2,3-octanediol | 0.1 |

TABLE 47

Antiperspirant stick

| Ingredients | INCI | Amount |
|---|---|---|
| PCL Liquid 100 | Cetearyl ethylhexanonate | ad 100 |
| Silicone Fluid 345 | Cyclomethicone | 10.0 |
| CRODACOL C90 | Cetyl Alcohol | 8.0 |
| SYNCROWAX HGLC | C18-36 Triglyceride | 8.0 |
| CRODAMOL PTC | Pentaerythritol Tetracaprylate/Caprate | 5.0 |
| SYNCROWAX HRC | Tribehenin | 4.0 |
| VOLPO N5 | Oleth-5 | 1.0 |
| Titanium Dioxide | Titanium Dioxide | 1.0 |
| Rezal 36GP | Aluminium Tetrachlorohydrex GLY | 20.0 |
| Dry Flo C | Aluminium Starch Octenyl Succinate | 22.5 |
| Preservative | Phenoxyethanol | 0.8 |
| Perfume oil PO1, PO2, PO3, PO4, or PO5 | Fragrance | 0.6 |
| 1,2-heptanediol/2,3-heptanediol blend (95:5 w/w %) | 1,2-heptanediol/2,3-heptanediol blend (95:5 w/w %) | 1.5 |

TABLE 48

Sunscreen lotion (O/W; broadband protection)

| | | |
|---|---|---|
| Ingredients | INCI | Amount |
| 1,2-heptanediol | 1,2-heptanediol | 0.15 |
| 1,2-octanediol/2,3-octanediol blend (95:5 w/w %) | 1,2-octanediol/2,3-octanediol blend (95:5 w/w %) | 0.5 |
| Carbopol Ultrez-10 | Carbomer | 0.2 |
| Dow Corning 246 Fluid | Cyclohexasiloxane and Cyclopentasiloxane | 2.0 |
| Dragosantol® 100* | Bisabolol | 0.3 |
| EDETA BD | Disodium EDTA | 0.1 |
| Emulsiphos® | Potassium Cetyl Phosphate, Hydrogenated Palm Glycerides | 1.5 |
| Perfume oil PO1, PO2, PO3, PO4, or PO5 | Fragrance | 0.4 |
| Frescolat®MGA | Menthone Glycerol Acetal | 0.3 |
| Glycerol 85% | Glycerin | 4.7 |
| Keltrol RD | Xanthan Gum | 0.2 |
| Lanette O | Cetearyl Alcohol | 1.0 |
| Neo Heliopan® 357 | Butyl Methoxy-dibenzoyl-methane | 1.0 |
| Neo Heliopan® AP (10% as sodium salt) | Disodium Phenyl Dibenzimidazole Tetrasulfonate | 10.0 |
| Neo Heliopan® AV | Ethylhexyl Methoxy-cinnamate | 3.0 |
| Neo Heliopan® Hydro (15% as sodium salt) | Phenylbenz-imidazole Sulfonic Acid | 6.7 |
| Neo Heliopan® MBC | 4-Methylbenzyl-idene Camphor | 1.5 |
| Neo Heliopan® OS | Ethylhexyl Salicylate | 5.0 |
| Neutral Oil | Caprylic/Capric Triglyceride | 2.0 |
| SymMatrix® | Maltodextrin. Rubus Fruticosus (Blackberry) Leaf Extract | 0.3 |
| SymOcide® BHO | Hydroxyacetophenone. Benzyl alcohol. Caprylyl glycol. Aqua | |
| Tegosoft TN | C12-15 Alkyl Benzoate | 5.0 |
| Tocopherol Acetate | Tocopheryl Acetate | 0.5 |
| Triethanolamine. 99% | Triethanolamine | 0.5 |
| Water (demineralized) | Water (Aqua) | ad 100 |

TABLE 49

Sun protection soft cream W/O; SPF 40

| Ingredients | INCI | Amount |
|---|---|---|
| Dehymuls PGPH | Polyglyceryl-2 dipolyhydroxystearate | 5.0 |
| Copherol 1250 | Tocopheryl acetate | 0.5 |
| Permulgin 3220 | Ozocerite | 0.5 |
| Zinc stearate | Zinc stearate | 0.5 |
| Tegosoft TN | C12-15 Alkyl benzoate | 10.0 |
| Neo Heliopan® E1000 | lsoamyl-p-methoxycinnamate | 2.0 |
| Neo Heliopan® 303 | Octocrylene | 5.0 |
| Neo Heliopan® MBC | 4-Methylbenzylidene camphor | 3.0 |
| Zinc oxide. neutral | Zinc oxide | 5.0 |
| Water. Distilled | Water (aqua) | ad 100 |
| EDETA BD | Disodium EDTA | 0.1 |
| Glycerol | Glycerol | 4.0 |
| Magnesium sulfate | Magnesium sulfate | 0.5 |
| Perfume oil PO1, PO2, PO3, PO4, or PO5 | Fragrance | 0.3 |
| Symdiol® 68 | Hexanediol, Caprylylglycol | 0.5 |
| 1,2-heptanediol | 1,2-heptanediol | 0.15 |

TABLE50

After sun balm

| Ingredients | INCI | Amount |
|---|---|---|
| SymSol® PF-3 | Water (Aqua), Pentylene Glycol, Sodium Lauryl Sulfoacetate, Sodium Oleoyl Sarcosinate, Sodium Chloride, Disodium Sulfoacetate, Sodium Oleate, Sodium Sulfate | 3.0 |
| Glycerol 99.5 P. | Glycerol | 5.0 |
| Sunbflower Oil | Helianthus Annuus (Sunflower) Oil | 1.0 |
| SymHelios® 1031 | Benzylidene Dimethoxydimethylin danone | 0.1 |
| Water | Water (Aqua) | ad 100 |
| Pemulen® TR-2 | Acrylates/C10-30 Alkyl Acrylate Crosspolymer | 1.0 |
| D-Panthenol 75 W | Panthenol | 0.5 |
| SymFinity® 1298 | Echinacea Purpurea Extract | 0.1 |
| Extrapone® Pearl GW | Water (Aqua), Glycerol, Hydrolyzed Pearl, Xanthan Gum | 1.0 |
| Sodium Hydroxide 10% solution | Sodium Hydroxide | 2.5 |
| Ethanol 96 % | Alcohol Denat. | 15.0 |
| Perfume oil PO1, PO2, PO3, PO4, or PO5 | Fragrance | 0.2 |
| SymOcide® PS | Phenoxyethanol. 1.2-Hexanediol. Decyleneglycol | 0.8 |
| 1,2-heptanediol | 1,2-heptanediol | 0.15 |
| 2,3-octanediol | 2,3-octanediol | 0.05 |

TABLE51

After sun lotion

| Ingredients | Amount |
|---|---|
| Acrylate/C10-30 alkylacrylate crosspolymer | 0.4 |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| Perfume oil PO1, PO2, PO3, PO4, or PO5 | 0.30 |
| 1,2-Hexanediol | 1.0 |
| 4-Hydroxyacetophenone | 0.3 |
| Pentylene glycol | 4.0 |
| Aqua dem. | ad 100 |
| Triethanolamine | 0.2 |
| 1,2-heptanediol | 0.2 |
| 2,3-octanediol | 0.1 |

TABLE 52

Syndet antimicrobial soap bar

| Ingredients | INCI | Amount |
|---|---|---|
| Zetesap 813 A | Disodium Lauryl Sulfosuccinate, Sodium Lauryl Sulfate, Corn Starch, Cetearyl Alcohol, Paraffin, Titanium Dioxide | ad 100 |
| Amphotensid GB 2009 | Disodium Cocoamphodiacetate | 6.0 |
| Allantoin | Allantoin | 1.0 |
| Perfume oil PO1, PO2, PO3, PO4, or PO5 | Fragrance | 1.0 |
| SymOcide C | o-cymen-5-ol | 0.1 |
| 1,2-heptanediol/2,3-heptanediol blend (95:5 w/w %) | 1,2-heptanediol/2,3-heptanediol blend (95:5 w/w %) | 5.0 |

TABLE 53

Syndet soap bar

| Ingredients | INCI | Amount |
|---|---|---|
| Fenopon AC-78 | Sodium Cocoyl Isethionate | 20.0 |
| Natrium laurylsulfoacetate | Sodium Lauryl Sulfoacetate | 16.0 |
| Paraffin | Paraffin | 19.0 |
| Wax. Microcrystalline | Microcrystalline Wax | 1.0 |
| Corn Starch | Corn Starch | 8.0 |
| Coconut acid | Coconut acid | 2.0 |
| Lauric acid diethanol amide | Lauramide DEA | 2.0 |
| Dextrin | Dextrin | 21.0 |
| Lactic acid. 88% | Lactic Acid | 1.0 |
| SymGuard CD | 3-Phenylpropanol, o-cymen-5-ol, Decylene glycol | 0.3 |
| Thymol | Thymol | 0.05 |
| Water | Water | ad 100 |
| Perfume oil PO1, PO2, PO3, PO4, or PO5 | Fragrance | 1.0 |
| 1,2-heptanediol/2,3-heptanediol blend (95:5 w/w %) | 1,2-heptanediol/2,3-heptanediol blend (95:5 w/w %) | 3.0 |

TABLE 54

Antimicrobial toilet soap bar

| Ingredients | Amount |
|---|---|
| Sodium soap from tallow | 60.0 |
| Sodium soap from palm oil | 27.0 |
| Glycerol | 2.0 |
| Sodium Chloride | 0.5 |
| 1-Hydroxyethane-1,1-diphosphonic acid, tetrasodium salt | 0.3 |
| Alpha-Tocopherol | 0.1 |
| Pigment Yellow 1 | 0.02 |
| Water | ad 100 |
| Perfume oil PO1, PO2, PO3, PO4, or PO5 | 3.0 |
| Farnesol | 0.2 |
| 2,3-heptanediol | 1.0 |
| 2,3-octanediol | 1.0 |

TABLE 55

Shaving foam

| Ingredients | Amount |
|---|---|
| Dem. Water | ad 100 |
| Triethanolamine | 4.0 |
| Edenor L2 SM (Stearinic acid. Palmitinic acid) (Cognis) | 5.3 |
| Laureth-23 | 3.0 |
| Stearylalcohol | 0.5 |
| SymOcide BHO (Hydroxacetophenone, Benzyl alcohol, Caprylyl glycol | 1.0 |
| 1,2-octanediol/2,3-octanediol blend (95:5 w/w %) | 0.3 |
| Sodium lauryl sulfate | 3.0 |
| Extrapone Seaweed (water, propylene glycol, potassium iodide Eucus Vesiculosus Extract) | 1.0 |
| Dragosantol (Bisabolol, Farnesol) | 0.1 |
| Perfume oil PO1, PO2, PO3, PO4, or PO5 | 1.0 |
| Propane, butane 4.2 Bar | 4.0 |

TABLE 56

Skin Oil

| Ingredients | INCI | Amount |
|---|---|---|
| Neutral Oil | Caprylic Capric Triglyceride | Ad 100 |
| Sunflower Oil | Sodium Lauryl Sulfoacetate | 20.0 |
| PCL Liquid 100 | Cetearyl Ethylhexanoate | 15.0 |
| Dragosantol 100 | Bisabolol | 0.2 |
| Vitamin E Acetate | Tocopheryl Acetate | 0.3 |
| Perfume oil PO1, PO2, PO3, PO4, or PO5 | Fragrance | 0.2 |
| 1,2-heptanediol/2,3-heptanediol blend (90:10 w/w %) | 1,2-heptanediol 2,3-heptanediol | 3.0 |

TABLE 57

Body, face and hair care oil

| Ingredients | INCI | Amount |
|---|---|---|
| Sweet Almond) Oil | Prunus Amygdalus Dulcis (Sweet Almond) Oil | 15.0 |
| Caprylic Capric Triglyceride | Caprylic Capric Triglyceride | Ad 100 |
| Tegosoft OP | Ethylhexyl Palmitate | 14.0 |
| Covasilk 15 | Silica Dimethyl Silylate | 1.0 |
| Isoadipate | Diisopropyl Adipate | 20.0 |
| Dragosantol 100 | Bisabolol | 0.2 |
| Vitamin E Acetate | Tocopheryl Acetate | 0.3 |
| Perfume oil PO1, PO2, PO3, PO4, or PO5 | Fragrance | 0.2 |
| 1,2-heptanediol | 1,2-heptanediol | 1.0 |
| 2,3-heptanediol | 2,3-heptanediol | 0.05 |

TABLE 58

Beard care

| Ingredients | INCI | Amount |
|---|---|---|
| Eutanol G | Octyldodecanol | 16.0 |
| Gelling Agent GP-1 | Dibutyl Lauroyl Glutamide | 2.4 |
| Gelling Agent EB-21 | Dibutyl Ethylhexanoyl Glutamide | 1.6 |
| Jojoba Oil | Simmondsia Chinensis (Jojoba) Seed Oil | 10.0 |
| Sweet Almond Oil | Prunus Amygdalus Dulcis (Sweet Almond) Oil | Ad 100 |
| SymClariol | Decylene Glycol | 0.1 |
| Dragoxat 89 | Ethylhexyl Isononanoate | 14.0 |
| Vitamin E Acetate | Tocopheryl Acetate | 0.3 |
| Perfume oil PO1, PO2, PO3, PO4, or PO5 | Fragrance | 0.2 |
| SymMollient PDCC | Propanediol Caprylate Caprate | 10.0 |
| Symlite G8 | Glyceryl Caprylate | 0.5 |
| Hydrolite-5 | Pentylene Glycol | 0.5 |
| 1,2-heptanediol | 1,2-heptanediol | 1.5 |
| 2,3-heptanediol | 2,3-heptanediol | 0.05 |

TABLE 59

Solid skin care concentrate

| Ingredients | INCI | Amount |
|---|---|---|
| Mango Butter | Mangifera Indica Seed Butter | Ad 100 |
| Carnauba Wax | Copernicia Cerifera (Carnauba) Wax | 6.0 |
| Rice Bran Wax | Oryza Sativa (Rice) Bran Wax | 6.0 |
| Cacao Butter | Theobroma Cacao (Cocoa) Seed Butter | 10.0 |
| ImerCare Pharma 00T | Talc | 2.0 |
| SymReboot L 19 | Maltodextrin. Lactobacillus Ferment | 0.5 |
| Perfume oil PO1, PO2, PO3, PO4, or PO5 | Fragrance | 0.2 |
| SymVital Age Repair 3040 | Zingiber Officinale (Ginegr) Root Extract | 0.2 |
| Sunflower Oil | Helianthus Annuus (Sunflower) Seed Oil | 20.0 |
| Symlite G8 | Glyceryl Caprylate | 0.5 |
| Argan Oil | Argania Spinosa Kernel Oil | 6.0 |
| 1,2-octanediol/2,3-octanediol blend (95:5 w/w %) | 1,2-octanediol/2,3-octanediol blend (95:5 w/w %) | 2.0 |
| 1,2-heptanediol | 1,2-heptanediol | 1.0 |

TABLE60

Solid shampoo

| Ingredients | INCI | Amount |
|---|---|---|
| Amisoft MS-11 | Sodium Myriostoyl Glutamate | Ad 100 |
| Elfan AT 84 | Sodium Cocoyl Isethionate | 15.0 |
| ImerCare 02K-S | Kaolin | 18.8 |
| Hydrolite-5 Green | Pentylene Glycol | 1.0 |
| Cetiol SB45 | Diisopropyl Adipate | 20.0 |
| SymDecanox HA | Bytyrrospermum Parkii Butter | 13.0 |
| Water | Water (Aqua) | 15.0 |
| Perfume oil PO1, PO2, PO3, PO4, or PO5 | Fragrance | 1.0 |
| 1,2-heptanediol/2,3-heptanediol blend (98:2 w/w %) | 1,2-heptanediol 2,3-heptanediol | 3.0 |

1-26. (canceled)

27. A cosmetic or pharmaceutical composition comprising:
(a) a mixture comprising at least one first linear alkanediol having a carbon chain of 5 to 14 carbon atoms and one or more second linear alkanediols having a carbon chain of 5 to 14 carbon atoms which is different from the first linear alkanediol;
(b) at least one liquid lipophilic component; and
(c) optionally at least one cosmetically or pharmaceutically active substance and/or additive;
wherein the mixture comprising at least one first linear alkanediol and one or more second linear alkanediols is selected from the group consisting of
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-octanediol and 2,3-octanediol;
a mixture comprising 1,2-nonanediol and 2,3-nonanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol;
a mixture comprising 1,2-tridecanediol and 2,3-tridecanediol;
a mixture comprising 1,2-hexanediol and 2,3-octanediol;
a mixture comprising 1,2-octanediol and 2,3-hexanediol; and
a mixture comprising 1,2-octanediol and 2,3-heptanediol.

28. A cosmetic or pharmaceutical composition according to claim 27, wherein the first linear alkanediol or the second linear alkanediol is an alkanediol having a maximum water solubility of less than or equal to 10% by weight.

29. A cosmetic or pharmaceutical composition according to claim 27, wherein the mixture comprising at least one first linear alkanediol and one or more second linear alkanediols comprises the first linear alkanediol and the second alkanediol in a ratio in a range of 50 : 50 to 99.9 : 0.1.

30. A cosmetic or pharmaceutical composition according to claim 27, wherein the at least one liquid lipophilic component is selected from the group consisting of plant oils, hydrocarbons, fatty alcohols, fatty acid esters, UV filters, and mixtures of two or more of the aforesaid liquid oil components.

31. A cosmetic or pharmaceutical composition according to claim 30, wherein the at least one liquid lipophilic component is a plant oil selected from the group consisting of Persea Gratissima (Avocado Oil), Argania spinosa kernel oil (Argan oil), Butyrospermum Parkii (Shea) Butter, Cocos Nucifera (Coconut) Oil, Glycine Soja (Soybean) Oil, Helianthus Annuus (Sunflower) Seed Oil, Olea Europaea (Olive) Fruit Oil, Prunus Amygdalus Dulcis (Sweet Almond) Oil, Ricinus Communis (Castor) Seed Oil, Simmondsia Chinensis (Jojoba) Seed oil, Theobroma Cacao (Cocoa) Seed Butter, Vitis Vinifera (Grape) Seed Oil, Caprylic Capric Triglycerides, and mixtures of two or more of the aforesaid plant oils; and/or
wherein the hydrocarbon is selected from the group consisting of undecane, tridecane, mineral oil, petrolatum, squalane, isohexadecane, C7 - C8 isoparaffin, C8 - C9 isoparaffin, C9 - C11 isoparaffin, C9 - C12 isoparaffin, C9 - C13 isoparaffin, C9 - C14 isoparaffin, C9 -C16 isoparaffin, C10 - C11 isoparaffin, C10 - C12 isoparaffin, C10 - C13 isoparaffin, C11 C12 isoparaffin, C11C13 isoparaffin, C11 - C14 isoparaffin, C12 - C14 isoparaffin, C12 C15 isoparaffin, C12C20 isoparaffin, C13 - C14 isoparaffin, C13 - C16 isoparaffin, C14 C16 isoparaffin, C15C19 isoparaffin, C18 - C70 isoparaffin, and mixtures of two or more of the aforesaid hydrocarbons; and/or
wherein the fatty alcohol is selected from the group consisting of phenyl propanol, dimethyl phenylbutanol, hexyldecanol, octyldodecanol, octyldecanol, tridecylalcohol, isostearyl alcohol, phenylisohexanol, phenylpropanol, trimethylbenzenepropanol, isoamylalcohol, isostearyl alcohol, isotridecyl alcohol, and mixtures of two or more of the aforesaid fatty alcohols; and/or
wherein the fatty acid ester is selected from the group consisting of C12 - C15 Alkyl Benzoate, Capric/Lauric/Myristic/Oleic Triglyceride, Caprylic/Capric Triglyceride, Caprylic/Capric/Lauric Triglyceride, Caprylic/Capric/Linoleic Triglyceride, Caprylic/Capric/Myristic/Stearic Triglyceride, Caprylic/Capric/Palmitic/Stearic Triglyceride, Caprylic/Capric/Stearic Triglyceride, Caprylic/Capric/Succinic Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethyl Succinate, Diethylhexyl 2,6-Naphthalate, Diethylhexyl Carbonate, Dibutyl Adipate, Diisopropyl Adipate, Dipropylheptyl Carbonate, Ethyl Laurate, Ethylhexyl Isononanoate, Ethylhexyl Palmitate, Ethylhexyl Stearate, Glyceryl Caprylate Caprate, Glyceryl Caprylate, Glyceryl Laurate, Glyceryl Triacetyl Hydroxystearate, Glyceryl Triacetyl Ricinoleate, Hexyl Laurate, Isoamyl acetate, Isoamyl Cocoate, Isopropyl Palmitate, Isosorbide Dicaprylate, Isopropyl Myristate, Myristyl Myristate, Oleic/Linoleic Triglyceride, Oleic/Palmitic Triglyceride, Oleic/Palmitic/Lauric/Myristic/Linoleic Triglyceride, Propanediol Caprylate, Ricinoleic/Caproic/Caprylic/ Capric Triglyceride, Oleostearine, Oleyl Erucate, Palmitic/Stearic Triglyceride, Propanediol Dicaprylate Caprate, Propylheptyl Caprylate, Stearyl Heptanoate/Stearyl Caprylate, Triheptanoin, Trihydroxystearin, Triisononanoin, Triisopalmitin, Triisostearin, Trilaurin, Trilinolein, Trilinolenin, Trimyristin, Triolein, Tripalmitin, Tripalmitolein, Tripelargonin, Triricinolein, and mixtures of two or more of the aforesaid fatty esters; and/or
wherein the UV filter is selected from the group consisting of Octocrylene, Ethylhexyl Methoxycinnamate, Homosalate, Ethylhexyl Salicylate, Isoamyl p-Methoxycinnamate, Camphor Benzalkonium Methosulfate, Polyarylamidomethyl Benzylidene Camphor, Isooctyl Methoxycinnamate, Ethylhexyl Triazone, Drometrizole Trisiloxane, Diethylhexyl Butamido Triazone, 3-Benzylidene Camphor, Octyl Salicylate, Ethylhexyl Dimethyl PABA, Bis-Ethylhexyloxyphenol Methoxyphenyl Triazine, Polysilicone-15, Diethylamino Hydroxybenzoyl Hexyl Benzoate, N,N,N-Trimethyl-4-(2-oxoborn-3-ylidenemethyl) anilinium methyl sulfate,
Benzoic acid-2-hydroxy-3,3,5-trimethylcyclohexyl ester/Homosalate,
2-Cyano-3,3-diphenyl acrylic acid, 2-ethylhexyl ester/Octocrilene,
2-Ethylhexyl-4-methoxycinnamate/Octinoxate,
Ethoxylated ethyl-4-aminobenzoate,
2-Ethylhexyl salicylate/Octisalate,
2-Ethylhexyl-4-(dimethylamino)benzoate/Padimate O (USAN:BAN),
2,2′-Methylene-bis(6-(2H-benzotriazol-2-yl)-4-(1,1,3,3-tetramethyl-butyl)phenol)/Bisoctrizole, Dimethicodiethylbenzalmalonate, and mixtures of two or more of the aforesaid UV sun protection filters.

32. A cosmetic or pharmaceutical composition according to claim 30, wherein the at least one liquid lipophilic component is a plant oil having a melting point in the range of -20° C. to ≤ 30° C.

33. A cosmetic or pharmaceutical composition according to claim 27, comprising
a mixture comprising 1,2-hexanediol and 2,3-hexanediol;
Glyceryl Caprylate; and
optionally at least one cosmetically or pharmaceutically active substance and/or additive.

34. A cosmetic or pharmaceutical composition according to claim 27, comprising
a mixture comprising 1,2-heptanediol and 2,3-heptanediol;
Glyceryl Caprylate; and
optionally at least one cosmetically or pharmaceutically active substance and/or additive.

35. A cosmetic or pharmaceutical composition according to claim 27, comprising
a mixture comprising 1,2-octanediol and 2,3-octanediol;
Glyceryl Caprylate; and
optionally at least one cosmetically or pharmaceutically active substance and/or additive.

36. A cosmetic or pharmaceutical composition according to claim 27, wherein the at least one cosmetically or pharmaceutically active substance and/or additive is selected from the group consisting of agents against ageing of the skin, anti-microbial agents, antioxidants, chelating agents, emulsifiers, surfactants, preservatives, green and synthetic polymers, skin-cooling agents, rheology additives, oils, fragrances or perfume oils, polyols, and mixtures of two or more of the aforementioned substances.

37. A cosmetic or pharmaceutical composition according to claim 27, comprising the mixture comprising at least one first linear alkanediol and one or more second linear alkanediols in an amount of 0.001 to 15.0% by weight.

38. A cosmetic or pharmaceutical composition according to claim 27,
comprising the at least one liquid lipophilic component in an amount of 0.01 to 50.0 % by weight based on the total weight of the composition, wherein the cosmetic or pharmaceutical composition is an emulsion; or
comprising the at least one liquid lipophilic component in an amount of ≥ 60% by weight based on the total weight of the composition, wherein the cosmetic or pharmaceutical composition is a water free formulation; or
comprising the at least one liquid lipophilic component in an amount of 1 to 40% by weight based on the total weight of the composition, wherein the cosmetic or pharmaceutical composition is a solid surfactant composition.

39. A cosmetic or pharmaceutical composition according to claim 27, wherein
the composition is a dispersion comprising the at least one liquid lipophilic component in an amount of ≥ 1% by weight, and wherein the cosmetic or pharmaceutical composition is in the form of an emulsion a liquid, a lotion, a suspension, a milk, an ointment, a paste, a gel, a cream based formulation, an oil based formulation or a liposome based formulation;
the composition is a water free formulation;
the composition is a solid surfactant composition; or
the composition is an aqueous or aqueous/alcoholic based solution.

40. A method of improving skin sensation of a liquid lipophilic component in a cosmetic or pharmaceutical composition upon topical application comprising formulating the composition as a composition of claim 27.
